(19) United States
(12) Patent Application Publication
(10) Pub. No.: US 2019/0391092 A1
(43) Pub. Date: Dec. 26, 2019

(54) METABOLIC PROFILING WITH MAGNETIC RESONANCE MASS SPECTROMETRY (MRMS)

(71) Applicant: Oregon Institute of Science and Medicine, Cave Junction, OR (US)

(72) Inventors: Arthur B. Robinson, Cave Junction, OR (US); Matthew L. Robinson, Cave Junction, OR (US); Noah Robinson, Cave Junction, OR (US)

(73) Assignee: Oregon Institute of Science and Medicine

(21) Appl. No.: 16/446,772

(22) Filed: Jun. 20, 2019

Related U.S. Application Data
(60) Provisional application No. 62/688,030, filed on Jun. 21, 2018.

(51) Int. Cl. G01N 24/08; H01J 49/00; H01J 49/16; G16H 50/30; G16H 10/40

(52) U.S. Cl. CPC G01N 24/087; H01J 49/0036; G16H 10/40; H01J 49/164; G16H 50/30; H01J 49/165

(57) ABSTRACT

A method for constructing a metabolic profile of a mammalian (such as a human) subject from one of more urine samples from the subject uses magnetic resonance mass spectrometry (MRMS) for the rapid and inexpensive quantitative measurement of at least 4,000 urinary chemical substances in a single analysis. The method for metabolic profiling measures thousands of urinary substances in a urine sample from a mammalian subject in a single assay. Many of these substances can be of mammalian metabolic origin. The measurements of types and amounts of urinary substances can be correlated to assessments of present or future health of the subject.

### CROSS-REFERENCE TO RELATED APPLICATIONS

This application claims the benefit of U.S. Provisional Application No. 62/688,030, filed Jun. 21, 2018, and incorporates that application by reference in its entirety.

This is a nonprovisional U.S. patent application filed under 37 C.F.R. § 1.53(b).

### STATEMENT REGARDING FEDERALLY SPONSORED RESEARCH OR DEVELOPMENT

Not applicable

### REFERENCE TO APPENDIX

The following appendix forms part of this application:

Appendix 1. N. Robinson, M. Robinson, and A. Robinson. Metabolic Profiling with Magnetic Resonance Mass Spectrometry and a Human Urine Bank: Profiles for Aging, Sex, Heart Disease, Breast Cancer and Prostate Cancer Journal of American Physicians and Surgeons, Volume 22, Number 3, Fall 2017, 75-84.

### TECHNICAL FIELD

The present invention relates to a method for measuring amounts of selected urinary substances of mammalian metabolic origin in a mammalian urine sample. The invention further relates to a method for monitoring changes in amounts of selected urinary substances of mammalian metabolic origin in a mammalian urine sample during a time period of interest. The invention also relates to a method for obtaining a metabolic profile from a urine sample from a subject using Magnetic Resonance Mass Spectrometry (MRMS).

### BACKGROUND OF THE INVENTION

Metabolic Profiling

The emphasis of the single-substance-orientated clinical chemistry industry is primarily upon diagnosis of overt disease by technologically inferior methods. Physicians are offered the amounts of single substances in human samples and comparisons with so-called normal values, which are typically two-standard-deviation ranges for the general population. Measurements of a couple of dozen such substances are included in ordinary analyses, and single substances beyond the normal range are noted and considered in patient evaluation. A large suite of additional single-substance measurements is available in industrial laboratories, which the physician can order to extend or confirm his diagnosis. This paradigm is expensive, so the number of substances measured is low and the application is limited to patients already exhibiting disease symptoms. Moreover, it entirely misses the metabolic patterns available from groups of substances that have values within the normal ranges—patterns that computer analysis can discover.

Quantitative metabolic profiling of analytically convenient metabolites allows a single analytical procedure, measuring a single large set of metabolites, to diagnose essentially all disease conditions with one inexpensive procedure (1). Moreover, by including computerized pattern recognition, metabolic profiling extracts far more complete and valuable medical information than does the traditional method.

The low cost and much greater information content of metabolic profiling permits its use in preventive medicine, allowing the individual to combat the probability of disease rather than overt disease itself, it also provides a convenient and inexpensive means of quantitative measurement of illness so that therapeutic procedures can be evaluated in real time—a capability almost entirely absent from current therapeutic medicine.

Moreover, physiological age can be quantitatively measured by metabolic profiling. This opens the way for evaluating the effects of various adjustable nutritional and other parameters on aging. This can allow single individuals to monitor their own rate of aging and probabilities of disease as a function of time and their own habits.

The low cost and therefore increased availability of health evaluation that metabolic profiling makes possible can save the lives of many people that are now lost because current methods—imbedded in an expensive, inconvenient health system and providing inferior information—fail to diagnose their illnesses in time.

“Health” is a concept that varies with individual objectives. Optimum health means different things to an athlete, to an artist, to a mathematician, or to a soldier. Each seeks to optimize different aspects of his or her abilities. The quantitative measurement of health that quantitative health profiling makes possible allows each person to optimize those abilities considered most valuable.

Mass Spectrometry

Mass spectrometry, including magnetic resonance mass spectrometry (MRMS), is currently in use, often in conjunction with chromatography, in searches for “biomarkers” for the diagnosis of human diseases. These are unusual single chemical substances or unusual amounts of ordinary single chemical substances in body fluids or tissues that are correlated with a current disease or a propensity for a disease. Biomarker methods reside primarily in clinical laboratories for use through physician-ordered tests. Taken as a group, biomarker tests—which are single condition and methodology specific—are inherently expensive and therefore mostly medical gatekeeper controlled.

Citation or identification of any reference in Section 2, or in any other section of this application, shall not be considered an admission that such reference is available as prior art to the present invention. All references cited herein are incorporated herein by reference in their entirety and for all purposes to the same extent as if each individual publication, patent or patent application was specifically and individually indicated to be incorporated by reference in its entirety for all purposes.

### REFERENCES

1. Robinson, A. B. and Robinson, N. E. (2011). Origins of Metabolic Profiling, in Metabolic Profiling, T. O. Metz (ed.), Methods in Molecular Biology 708, 1-23, Springer Science+Business Media, LLC, DOI 10.1007/978-1-61737-985-7_1.

### SUMMARY OF THE INVENTION

A method for constructing a metabolic profile is provided. A metabolic profile is a list of substances that correlate with a disease or condition. For example, the substances can be produced by a subject or recoverable or sampled from a subject's body, such as body fluids (e.g., urine) or breath. In an embodiment, the method comprises:

obtaining a urine sample from a mammalian subject (e.g., a human patient);

diluting the sample solely with ultra-pure water to a concentration suitable for mass spectrometry;

obtaining a mass spectrum of the urine sample, wherein obtaining the mass spectrum comprises:

subjecting the urine sample to magnetic resonance mass spectrometry (MRMS),

determining mass spectrometric peaks in the mass spectrum present in analytically significant amounts,

identifying, among the plurality of mass spectrometric peaks in the mass spectrum present in analytically significant amounts, mass spectrometric peaks representing a plurality of urinary substances of interest, wherein identifying the plurality of urinary substances of interest comprises:

identifying all mass spectrometric peaks present in analytically significant amounts in the mass spectrum that represent:
each urinary substance of interest in the plurality,
each isotope of each urinary substance of interest in the plurality,
each adduct (e.g., salt) of each urinary substance of interest in the plurality, and/or
each variant of each urinary substance of interest in the plurality;

quantitatively measuring the amount of each urinary substance of interest in the plurality by summing, for each urinary substance of interest in the plurality, the mass spectrometric peaks representing:
each urinary substance of interest in the plurality,
each isotope of each urinary substance of interest in the plurality,
each adduct (e.g., salt) of each urinary substance of interest in the plurality, and/or
each variant of each urinary substance of interest in the plurality; and

performing statistical calculations to determine a diagnostically useful profile by determining what combinations and/or amounts of urinary substances of interest in the plurality correlate with a disease or condition of interest, thereby constructing a metabolic profile of the disease or condition of interest in the subject.

In an embodiment of the method, the mammalian subject is a human. In other embodiments, the mammalian subject is a domestic animal (e.g., cat, dog, cow, horse, sheep, pig, goat, etc.) or a rodent (e.g., rat or mouse).

In an embodiment of the method, MRMS is performed in positive ion mode.

In an embodiment of the method, the MRMS comprises laser desorption ionization (LDI).

In an embodiment of the method, the MRMS is matrix assisted laser desorption ionization (MALDI)-MRMS.

In an embodiment of the method, the urinary substance of interest is of mammalian (e.g., human) metabolic origin.

In an embodiment, the method further comprises identifying the plurality of substances of human metabolic origin present in the urine sample that are also present in at least 80% of metabolic profiles in a database of metabolic profiles of human urine samples.

In an embodiment of the methods, the urine sample is introduced onto a nanopost array ionization plate (or a nanopost matrix) and the LDI is performed from the nanopost array ionization plate. In other embodiments, LDI is carried out on any suitable clean fabric or substrate.

In an embodiment of the method, the MRMS is electrospray (ESI)-MRMS.

In an embodiment of the method, the sample is diluted with ultra-pure water only (and no other substance).

In an embodiment of the method, the urinary substance of interest is of mammalian (e.g., human) metabolic origin.

In an embodiment of the method, mass spectral analysis is performed over a range from 75 to 1,000 m/z.

In an embodiment of the method, at least one of the urinary substances of interest in the plurality is selected from the urinary substances listed in Table 2.

In other embodiments, some or all of the urinary substances of interest in the plurality are selected from the urinary substances listed in Table 2.

In an embodiment of the method, the volume of the urine sample is 0.1-1 μl, 1-10 μl, 10-20 μl, 20-30 μl, 30-40 μl, or 40-50 μl. In another embodiment of the method, the volume of the urine sample is 5 μl.

In an embodiment of the method, the urine sample is subjected to a run of magnetic resonance mass spectrometry (MRMS) in positive ion mode of a duration of 0.1 min-1 min, 1 min-5 min, 5 min-10 min, 10 min-30 min, or 30 min-60 min.

In an embodiment of the method, the urine sample is subjected to a series of runs of magnetic resonance mass spectrometry (MRMS) in positive ion mode of a total duration of 2 min.

In an embodiment of the method, the mass spectrometric peaks with specific molecular masses present in analytically significant amounts in the urine sample are identified by their exact masses as known in the art.

In an embodiment of the method, the disease or condition of interest is cardiovascular disease, cardiac illness, prostate cancer or breast cancer.

In an embodiment of the method, the disease or condition of interest is selected from a disease or condition of interest listed in Table 1.

A method is also provided for assessing the progression of a disease or condition of interest in a mammalian (e.g., human) patient during a time period of interest comprising:

obtaining a metabolic profile from a urine sample from the human patient at selected sequential time points in the time period of interest;

determining the amounts of:

urinary substances of interest for monitoring for the progression of a disease of interest in its metabolic profile;

calculating the change in amount of urinary substances of interest among each of the selected sequential time points in the time period of interest;

calculating, with successive strength of each metabolic profile obtained at a selected sequential time point in the time period of interest, the progress of the patient's illness as a function of time and treatment, wherein the calculating comprises determining a diagnostic coefficient for the condition of interest:

determining:

which parameters are indicative that the disease is progressing in the patient;

which parameters are indicative that the disease is not progressing in the patient;

which parameters are indicative that the disease is diminishing or that the patient's health is improving, and

if the disease is progressing in the patient, administering a drug or treatment to ameliorate, reverse or stop the progression of the disease; or

if the disease is not progressing in the patient, modulating therapy appropriately.

In an embodiment of the method, the mammalian patient is a human patient.

In an embodiment of the method, the disease or condition of interest is cardiovascular disease, cardiac illness, prostate cancer, or breast cancer. In another embodiment of the method, the disease or condition of interest is selected from a disease or condition of interest listed in Table 1.

A method is provided for assessing the presence and amounts of at least one urinary substance of interest in a mammalian urine sample, the method comprising:

obtaining a urine sample from a mammalian patient (e.g., a human patient);

diluting the sample solely with ultra-pure water to a concentration suitable for mass spectrometry;

obtaining a mass spectrum of the urine sample, wherein obtaining the mass spectrum comprises:

subjecting the urine sample to magnetic resonance mass spectrometry (MRMS),

identifying mass spectrometric peaks in the mass spectrum present in analytically significant amounts, wherein the identifying mass spectrometric peaks comprises:
performing a statistical evaluation to demonstrate existence of a metabolic profile, and
testing the metabolic profile by a diagnostic coefficient method,

identifying at least one urinary substance of interest among a plurality of urinary substances in the urine sample, wherein identifying the at least one urinary substance of interest comprises identifying all mass spectrometric peaks in the mass spectrum representing the at least one urinary substance of interest, isotopes of the at least one urinary substance of interest, adducts (e.g., salts) of the at least one urinary substance of interest, and/or other variants of the at least one urinary substance of interest;

quantitatively measuring the amount of the at least one urinary substance of interest by summing the mass spectrometric peaks in the plurality comprising:

identifying the isotopic peak of all molecular ions of the urinary substance of interest,

identifying the isotopic peak of all molecular ion adducts of the urinary substance of interest,

identifying the isotopic peaks of a molecular ion variants of the urinary substance of interest,

combining these peaks to determine the amount of the urinary substance of interest.

In an embodiment of the method, the mammalian patient is a human patient.

In an embodiment of the method, MRMS is performed in positive ion mode (LDI).

In an embodiment of the method, the MRMS is matrix assisted laser desorption ionization (MALDI)-MRMS.

In an embodiment of the method, the urinary substance of interest is of mammalian (e.g., human) metabolic origin.

In an embodiment, the method further comprises identifying the plurality of substances of human metabolic origin present in the urine sample that are also present in at least 80% of metabolic profiles in a database of metabolic profiles of human urine samples.

In an embodiment of the method, the urine sample is introduced onto a nanopost array ionization plate (or a nanopost matrix) and the LDI is performed from the nanopost array ionization plate. In other embodiments, LDI is carried out on any suitable clean fabric or substrate.

In an embodiment of the method, the MRMS is electrospray (ESI)-MRMS.

In an embodiment of the method, the sample is diluted with ultra-pure water only (and no other substance).

In an embodiment of the method, the urinary substance of interest is of mammalian (e.g., human) metabolic origin.

In an embodiment of the method, mass spectral analysis is performed over a range from 75 to 1,000 m/z.

In an embodiment of the method, a plurality of urinary substances of interest are assessed. In another embodiment of the method, the urinary substances of interest in the plurality are selected from the urinary substances listed in Table 2.

In an embodiment of the method, the volume of the urine sample is 0.1-1 μl, 1-10 μl, 10-20 μl, 20-30 μl, 30-40 μl, or 40-50 μl.

In an embodiment of the method, the volume of the urine sample is 5 μl.

In an embodiment of the method, the urine sample is subjected to a run of magnetic resonance mass spectrometry (MRMS) in positive ion mode of a

In an embodiment of the method, the urine sample is subjected to a series of runs of magnetic resonance mass spectrometry (MRMS) in positive ion mode of a total duration of 2 min.

In an embodiment of the method, the mass spectrometric peaks with specific molecular masses present in analytically significant amounts in the urine sample are identified by their exact masses as known in the art.

Methods disclosed herein can also be used to make heath assessments or predictions. In an embodiment, a change in amount of at least one urinary substance of interest in a mammalian urine sample can be monitored during a time period of interest. A urine sample is obtained from a mammalian patient (e.g., a human patient). A plurality of sequential time points are selected at which to measure an amount of a selected urinary substance of interest in the urine sample. The method for constructing a metabolic profile is performed at a first selected time point at the beginning of the time period of interest and at each of the selected subsequent sequential time points in the time period of interest. Changes in amounts of the at least one urinary substance of interest are calculated for each sequential time point of the plurality of sequential time points during the selected time period by comparing the amount of the at least one selected urinary substance of interest at the first selected time point to the amount of the at least one selected urinary substance of interest at the selected subsequent sequential time points of the plurality. This calculation, as disclosed herein, comprises determining a diagnostic coefficient.

### BRIEF DESCRIPTION OF THE DRAWINGS

Embodiments are described herein with reference to the accompanying drawings, in which similar reference characters denote similar elements throughout the several views. It is to be understood that in some instances, various aspects of the invention/embodiments may be shown exaggerated, enlarged, exploded, or incomplete to facilitate an understanding of the invention.

FIGS. 1A-1B. Actual and potentially enhanced U.S. survival. The line plotted in (a) and (b) is the U.S. survival curve. Physiologically calibrated MRMS of urine from a mammalian subject (e.g., human subject) can be used to produce a metabolic profile of the subject. A “metabolic profile” is a list of identified substances in a sample (e.g., urine sample) from a subject that correlates with a disease or condition of interest. For example, some substances might increase in quantity with (for example) the probability of getting a heart attack, while others might decrease. Physiologically calibrated MRMS of urine could prevent much of this suffering and early death as illustrated in (b) by enabling early diagnosis and preventive treatment.

FIG. 2. Use of diagnostic coefficients. The empirical determination of the positions, designated X, of single individuals on these illustrative linear axes by means of metabolic profiling available to everyone at low cost can facilitate lifestyle and medical intervention on their behalf. Research with such profiles on groups of individuals helps to guide those interventions.

FIGS. 3A-3B. Age distribution of 5,000 contributors to Oregon Institute of Science and Medicine (OISM) urine sample bank. Men (a) and women (b) from southern and central Oregon, U.S., volunteered to contribute urine samples and medical information to the urine bank.

FIGS. 4A-4B. Cumulative distribution function of nonparametric probability of non-correlation, P, of MRMS-measured urinary peaks with sex and age. The peaks for sex used age-matched controls, and those for age used sex-matched controls. The lower gray line in each graph is the theoretical plot for non-correlated measurements.

FIG. 5. Diagnostic power graph. This shows the accuracy of classifying men as “older” (above chronological age 50) or “younger” (below age 50) by metabolic profiling. Note that the measurement is of physiological age.

FIGS. 6A-6C. Cumulative distribution functions of nonparametric probability of non-correlation of MRMS-measured substances in urine provided pre-diagnosis: (a) the first analysis of cardiac events; (b) breast cancer; (c) prostate cancer.

FIGS. 7A-7D. Diagnostic Separations of subjects versus age and sex-matched controls: (a) cardiac events in first analysis; (b) cardiac events in second analysis; (c) breast cancer; (d) prostate cancer. Probability of correlation is 99.5%, 99.8%, 94%, and 97%, respectively.

FIG. 8. Cardiac event diagnostic coefficients for 200 men and women with no known health problems. It is shown that 28% of these people have positive diagnostic coefficients. About 27% of the U.S. population in the age distribution of the 200 are actuarially expected to eventually die from heart disease.

FIG. 9. Diagnostic power graph of cardiac event prediction by MRMS of urine for 21 subjects.

FIG. 10. A mass spectrum of the urine of a 93-year old human subject in the m/z 260.7 to 261.3 region. The complete MRMS mass spectrum between 75 and 1,000 amu contains 925 such regions. On average, about 200 peaks representing molecules with different m/z are detected in each such region, and we used about 35 of these in our profiles.

### DETAILED DESCRIPTION OF THE INVENTION

The inventors disclose herein a method for quantitative metabolic profiling. The inventors have discovered that many human metabolites (substances made in the course of human metabolism), as well as other substances present in human physiological fluids and tissues that are not human metabolites, contain small amounts of measurable information useful for the quantitative measurement of human health. The inventors have discovered that simultaneous measurement of these substances can be performed at low cost with the method disclosed herein. This method can revolutionize the quantitative measurement of human health for many medical and other desirable purposes.

The inventors have discovered that the method for metabolic profiling disclosed herein meets the following criteria for quantitatively assessing human health and making future predictions about human health. A “metabolic profile” is a list of substances that correlate with a disease or condition. Some substances might increase in quantity with (for example) the probability of getting a heart attack, while others decrease.

First, the method for metabolic profiling measures large numbers of substances, since the amount of information expected from each is, on average, small.

Second, a biochemical understanding of the measured substances (even, in the limit, knowledge of their chemical identities) is not necessary, since development and use of this method is entirely empirical.

Third, the method for metabolic profiling measures the quantities of the substances.

Fourth, the distribution functions of the measured substances in the human population need not be known initially, but until known, all statistical analysis are nonparametric.

Fifth, functional forms used in analysis of the data can be enhanced by known biochemical principles. In the method disclosed herein, logarithms of the measured values and ratios of those logarithms have been discovered to be of value. Data analysis reduces the measurements to practical, useful parameters, especially linear decision-capable arrays.

Sixth, the method can be applied to most human biochemical conditions. The Examples disclosed herein demonstrate positive evidence, since all five conditions—age, sex, impending heart attacks, impending breast cancer symptoms, and impending prostate cancer problems—were found to have unique and useful metabolic profiles.

Seventh, the method is quick and low cost, making it available to all people for use in life style optimization and preventive medicine as well as ordinary medical practices.

Eighth, in embodiments of the method, individuals can serve as their own controls in order to eliminate noise from genetic and life experience differences. This has been accomplished with a human sample bank (“urine bank”) as disclosed herein.

For clarity of disclosure, and not by way of limitation, the detailed description of the invention is divided into the subsections set forth below.

Method for Metabolic Profiling of Urine Samples with Magnetic Resonance Mass Spectrometry (MRMS)

A method is provided for constructing a metabolic profile (also referred to herein as “metabolic profiling”) of a mammalian subject from one of more urine samples from the subject using magnetic resonance mass spectrometry (MRMS). In an embodiment of the method, the mammalian subject is a human. In other embodiments, the mammalian subject is a domestic animal (e.g., cat, dog, cow, horse, sheep, pig, goat, etc.) or a rodent (e.g., rat or mouse).

The method uses magnetic resonance mass spectrometry (MRMS), for the simultaneous, rapid and inexpensive quantitative measurement of at least 4,000 urinary chemical substances (also referred to herein as “urinary (or urine) substances” or “urinary (or urine) constituents”). In an embodiment of the method, the MRMS uses laser desorption ionization (LDI) in positive ion mode. In another embodiment of the method, embodiment of the method, the MRMS uses laser desorption ionization (LDI) in negative ion mode.

The method disclosed herein overcomes many of the informational and financial limitations of biomarker methods. In one embodiment, the method for metabolic profiling measures thousands of urinary substances in a urine sample from a mammalian (e.g., human) subject using a single analysis or assay. Many of these substances can be of mammalian (e.g., human) metabolic origin. The measurements of types and amounts of urinary substances can be correlated to assessments of present and/or future health of the subject.

The method for metabolic profiling comprises obtaining a mass spectrum of a urine sample from a mammalian subject (e.g., human subject, also referred to herein as “patient”), or to obtain a plurality of urine samples from the subject obtained at periodic intervals of interest. The sample(s) are analyzed with reference to mass spectra for urine samples obtained at periodic intervals from thousands of human subjects (at least 5,000 subjects in Examples 1 and 2). In an embodiment of the method, the method is used to analyze a collection of urine samples, such as human urine samples in a “urine bank.” This analysis, also referred to herein as “metabolic profiling,” can be used to assess the present and/or future health of the subject. A metabolic profile is a set of substances whose relative measured amounts have been found to correlate with a disease or other condition: some substance amounts/or ratios increasing in amount with the condition and some decreasing in amount with the condition.

In an embodiment of the method, the MRMS characteristics of some or all of the at least 4,000 urinary chemical substances obtained from a subject's urine sample are used to construct metabolic profiles for quantitative measurement and assessment of present and/or future health of the subject. In another embodiment of the method, the MRMS characteristics of 5-10, 10-50, 50-100, 100-200, 200-300, 300-400, 400-500, 500-600, 600-700, 700-800, 800-900, or 900-1000 urinary chemical substances are used to construct metabolic profiles. The inventors have found that analyzing the MRMS characteristics of a subset of 100-800 urinary chemical substances produces extremely reliable diagnostic or predictive metabolic profiles. Table 2 sets forth a list of 833 urinary chemical substances. The inventors have found that about 700 of these substances met the criteria of appearing in 80% of the samples in each profiling experiment.

The method can be used to monitor a change in a substance of interest in a metabolic profile obtained from a mammalian (e.g., human) urine sample over a selected sequence of time points or during a selected time period. A series of urine samples is obtained from a subject at selected time points wherein the plurality of time points comprises at least a first selected time point and a second, later, selected time point. A quantitative change in strength of the selected metabolic profile during the selected time period is calculated by comparing the first selected time point to the later selected time point or points by means of computer software that looks to see if a substance or ratio of substances statistically changes systematically with time.

Sequential profiles to evaluate time-dependent health conditions utilizing these sequential profiles to obtain baseline profiles for the subject that are characteristic of the subject's biochemical and experiential individuality. This markedly enhances the value of the subject's current and future profiles by making possible the use of the subject as the subject's own control, rather than comparing the subject to a population of subjects.

The method disclosed herein can be applied to urine samples obtained repetitively from human subjects, who can be individual members of the public in all walks of life and conditions of health. Urine samples from these subjects are stored cryogenically in the urine bank. The mass spectrum associated with each urine sample, as well as health information about the donor, are stored in a database (e.g., a computer database). Periodic samples donated over time from a plurality of donors are stored in the urine bank and continue to expand the urine bank's database with their associated mass spectra and donor health information. This method has broad practical applications and is a powerful approach to health analysis and prediction. In an embodiment, a donor or patient can serve as his or her own control and thereby escape comparisons to a genetically and experientially diverse general public

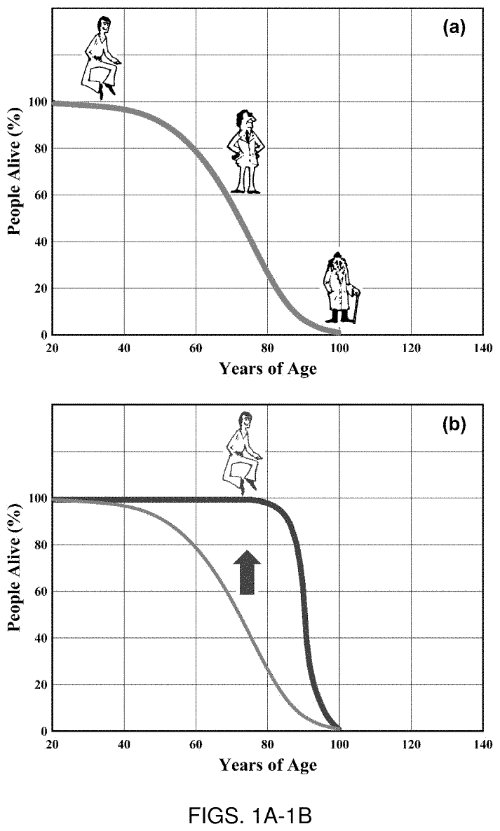

FIGS. 1A-1B shows actual and potentially enhanced U.S. survival. The line plotted in (a) and in (b) is the U.S. survival curve. Physiologically calibrated MRMS of urine from a mammalian subject (e.g., human subjection) can be used to produce a metabolic profile of the subject. Such calibration involves the measurement of metabolic profiles in humans as functions of their present and future health. Using MRMS, patterns of distribution and amounts of substances of interest are identified in urine samples for each disease of interest. Information obtained about the amounts of substances of interest is measured in urine, and against a set of diseased and control samples.

Future health calibration involves the measurement of metabolic profiles in cryogenically preserved urine samples provided by a subject in the historical past before the health aspect (e.g., disease or health condition) of interest was manifested. Urine samples from the subject before s/he manifested the health aspect (e.g., disease or health condition), along with urine samples from other donors, can be used as controls. The metabolic profile of the subject can be employed in calculations of present and predicted future health to prevent much of this suffering and early death as illustrated in (b) by enabling early diagnosis and preventive treatment.

In an embodiment, a method is provided for obtaining a metabolic profile from a urine sample from a human subject, wherein the metabolic profile comprises at least a first mass spectrometric peak in a mass spectrum representing a first urinary substance of human metabolic origin and a second mass spectrometric peak in a mass spectrum representing a second urinary substance of human metabolic origin and thousands of additional mass spectrometric peaks.

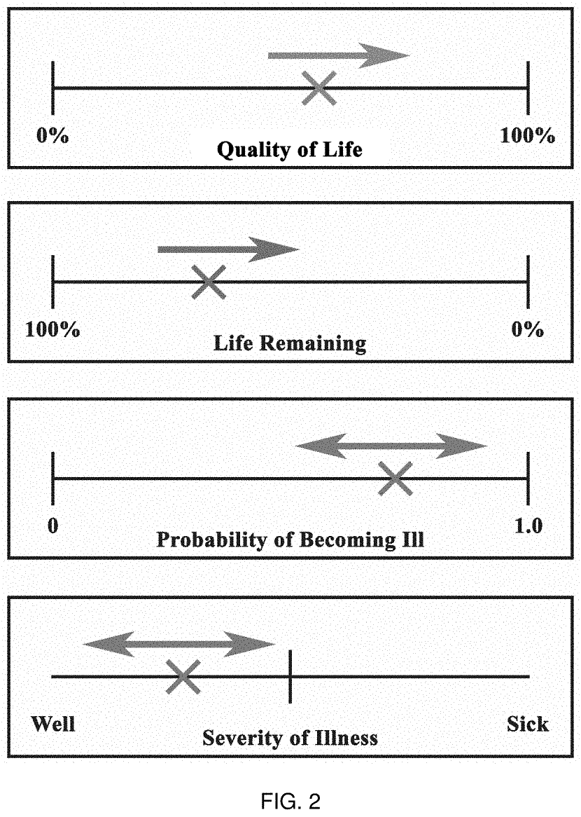

In an embodiment, a method is provided for assessing the progression of a disease in a patient and/or for optimizing the patient's medical treatment, the method comprising: obtaining a first metabolic profile from a urine sample from a human patient at a first selected time point; determining the metabolic profile for the patient's illness; obtaining a second metabolic profile from urine sample from a human patient at a second selected time point; and then calculating the position on the diagnostic line (called the diagnostic coefficient R_{A}) as shown in FIG. 2, which is determined with the relative quantitative correlation with the well and sick profiles, for the progression of the patient's illness as a function of time and treatment. Information obtained from this calculation of the progression of the patient's illness can be used to modify or optimize the patient's medical treatment.

Diagnostic coefficients R_{A} are defined as:

$${R}_{A} = \frac{100}{{\sum }_{i = 1}^{n} {r}_{i}} [ {\sum }_{i = 1}^{n} \frac{\vert {A}_{i} - {Y}_{i} \vert {r}_{i}}{{A}_{i} + {Y}_{i}} - {\sum }_{i = 1}^{n} \frac{\vert {A}_{i} - {O}_{i} \vert {r}_{i}}{{A}_{i} + {O}_{i}} ]$$

where Ai is the normalized value of the ith parameter in the mass spectrum, A, that is being classified. Yi and Oi are the average values of the corresponding parameters in the two groups being compared, n is the number of parameters in the calculation, and ri is a weight constant that was set equal to 1 for all parameters in the calculations herein for simplicity in evaluating these results.

The method has already proved accurate in predicting presence of, or propensity for, cardiovascular disease, cardiac illness, prostate cancer and breast cancer. The method can be applied to other diseases, conditions, and states of health in individual subjects.

The method is robust because it can quantitatively measure essentially all health conditions in an individual simultaneously by means of one low-cost measurement. The method can be used to detect patterns in metabolic profiles characteristic of future heart attacks, breast cancer, and prostate cancer in people with no present indications of these illnesses. The method can also be used to construct metabolic profiles for specific physiological age and sex of a subject.

Example 1 demonstrates the practical diagnostic power of the method for the prediction of heart attacks. Applying the method to analyze urine samples obtained repetitively from subjects with varying conditions of health, banking their urine samples by storing them cryogenically and also storing health information obtained repetitively from the subjects donating the urine samples, enables subjects to serve as their own controls instead of being comparisons to the compared with a genetically and experientially diverse general public as a whole.

The method disclosed herein can be used to analyze, diagnose and treat the conditions of health or diseases listed in Table 1.

TABLE 1
Diseases or
conditions of interest

Alzheimer's disease
Anotia
Anthrax
Appendicitis
Arthritis
Aseptic meningitis
Asthenia
Asthma
Atherosclerosis
Athetosis
Bacterial meningitis
Beriberi
Breast cancer
Bronchitis
Bubonic plague
Calculi
Cancer
Cataracts
Cervical Cancer
Celiac disease
Cerebral palsy
Chagas disease
Chickenpox
Cholera
Chordoma
Chorea
Chronic fatigue syndrome
Circadian rhythm sleep
disorder
Chronic obstructive
pulmonary disease
(COPD)
Coccidioidomycosis
Colitis
Colon Cancer
Common cold
Condyloma
Congestive heart disease
Coronary heart disease
Cretinism
Crohn's Disease
Dengue
Diabetes
Diphtheria
Dysentery
Ear infection
Encephalitis
Emphysema
Epilepsy
Fibromyalgia
Gangrene
Gastroenteritis
Gastroesophageal reflux
disease (GERD)
Goiter
Gonorrhea
Heart disease
Hepatitis A
Hepatitis B
Hepatitis C
Hepatitis D
Hepatitis E
Histiocytosis
High Blood Pressure
Human papillomavirus
Huntington's disease
Hypermetropia
Hyperopia
Hyperthyroidism
Hypothyroid
Hypotonia
Impetigo
Infertility
Influenza (“flu”)
Interstitial cystitis
Iritis
Iron-deficiency anemia
Irritable bowel syndrome
Ignious Syndrome
Jaundice
Keloids
Kidney Disease
Kidney stones
Kwashiorkor
Laryngitis
Lead poisoning
Legionellosis
Leishmaniasis
Leprosy
Leptospirosis
Leukemia
Listeriosis
Liver Disease
Loiasis
Lung cancer
Lupus erythematosus
Lyme disease
Lymphogranuloma
venereum
Lymphoma
Limbtoosa
Liver Disease
Malaria
Marburg fever
Measles
Melanoma
Metastatic cancer
Ménière's disease
Meningitis
Migraine
Mononucleosis
Multiple myeloma
Multiple sclerosis
Mumps
Muscular dystrophy
Myasthenia gravis
Myelitis
Myoclonus
Myopia
Myxedema
Morquio Syndrome
Mattticular syndrome
Mononucleosis
Multiple Sclerosis
Muscular Dystrophy
Neoplasm
Non-gonococcal urethritis
Necrotizing Fasciitis
Osteoarthritis
Osteoporosis
Otitis
Oral Cancer
Ovarian Cancer
Palindromic rheumatism
Pancreatitis
Pancreatic Cancer
Paratyphoid fever
Parkinson's disease
Pelvic inflammatory
disease
Peritonitis
Periodontal disease
Pertussis
Phenylketonuria
Plague
Poliomyelitis
Porphyria
Progeria
Prostatitis
Prostate Cancer
Psittacosis
Psoriasis
Pulmonary embolism
Pilia
Pneumonia, Viral
Pneumonia, Bacterial
Rabies
Rectal Cancer
Rheumatism
Rheumatoid arthritis
Rickets
Rift Valley fever
Rocky Mountain spotted
fever
Rubella
Salmonellosis
Scabies
Scarlet fever
Sciatica
Scleroderma
Scrapie
Scurvy
Sepsis
Septicemia
SARS
Shigellosis
Shin splints
Shingles
Sickle-cell anemia
Siderosis
Silicosis
Smallpox
Stevens-Johnson syndrome
Stomach flu
Stomach ulcers
Stomach Cancer
Stroke
Strabismus
Strep throat
Streptococcal infection
Stroke
Sudden Infant Death
Syndrome (SIDS)
Synovitis
Syphilis
Swine influenza
Schizophrenia
Taeniasis
Tay-Sachs disease
Teratoma
Tetanus
Thalassaemia
Thrush
Thymoma
Thyroid Disease
Tinnitus
Tonsillitis
Tooth decay
Toxic shock syndrome
Trichinosis
Trichomoniasis
Trisomy
Tuberculosis
Tularemia
Tungiasis
Typhoid fever
Typhus
Tumor
Ulcerative colitis
Ulcers
Uremia
Urticaria
Urinary Cancer
Uterine Cancer
Uveitis
Varicella
Varicose veins
Vasovagal syncope
Vitiligo
Von Hippel-Lindau
disease
Viral fever
Viral meningitis
Warkany syndrome
Warts
Watson Syndrome
Yellow fever
Yersiniosis

Magnetic Resonance Mass Spectrometry (MRMS) of Urine Samples

In an embodiment, a mass spectrum is obtained of a urine sample provided by a donor or patient as follows. Fresh or thawed urine samples are subjected to concentration normalization with ultrapure water. The dilution can be from zero dilution to 100:1 parts water: urine sample. Ultrapure water is defined as 99.999% or purer water. After the concentration of the urine sample is normalized with ultrapure water, the urine sample is subjected to magnetic resonance mass spectrometry (MRMS) in positive ion mode. Magnetic resonance mass spectrometry (MRMS) in positive ion mode is well known in the art. Any method of MRMS known in the art can be used. In an embodiment of the method, the MRMS uses laser desorption ionization (LDI) in positive ion mode. In another embodiment of the method, the MRMS uses LDI in negative ion mode. Both these methods are well known in the art. In another embodiment, the MRMS uses electrospray (ESI), which is also well known in the art.

MRMS can measure thousands of substances quickly, inexpensively, and quantitatively. MRMS utilizes the extremely high resolution and high mass measurement accuracy of Fourier transform ion cyclotron resonance (FTICR) mass spectrometry. This high resolution allows thousands of independent chemical substances to be detected and quantified in a single analysis for a single sample without the requirement for prior separation. This provides the ability to discern the extensive: metabolite information generated from the ionization of urine samples, while providing a unique speed advantage. Moreover, the molecular formulae for most of these signals can be confirmed by accurate mass measurement, providing great specificity.

30,000 or more mass spectrometric peaks can be analyzed using the method. In embodiments, at least 30,000, at least 40,000 or at least 50,000 mass spectrometric peaks are analyzed. These spectrometric peaks represent urinary substances, i.e., substances of unique mass in the mass spectrum that are usually present in urine and are present in analytically significant amounts. These urinary substances are identified using customized computer software that locates and measures the peaks.

In embodiments, analyzing a urine sample by MRMS does not comprise at least one of the following techniques: performing a routine method of chromatography on the urine sample, transferring the urine sample through tubing, desalting the urine sample, and addition of laser matrix enhancers to the urine sample. Introduced impurities are sufficiently depressed so that in an embodiment of the method, at least 100,000 substances with distinct masses can be detected in a single assay or analysis using MRMS and at least 30,000 of these substances can be quantitatively measured using MRMS.

In an embodiment, a portion of a urine sample to be assayed is dried onto a nanopost array ionization plate and the LDI is performed from the nanopost array ionization plate. Alternatively, a portion of the urine sample to be assayed is dried on a suitable, clean surface, e.g., clean fabric.

In embodiments, the volume of the portion of the urine sample to be assayed is 0.1-1 μl, 1-10 μl, 10-20 μl, 20-30 μl, 30-40 μl, or 40-50 μl. In a specific embodiment, the volume to be assayed is 5 μl.

In an embodiment, the MRMS analysis is performed over a range from 75 to 1,000 m/z.

In an embodiment, the urine sample is subjected to a series of runs of magnetic resonance mass spectrometry (MRMS) in positive ion mode of a total duration of 2 minutes.

Urinary profiles are constructed using MRMS information from about 1,000 urinary substances known in the art to arise in human metabolism or, alternatively from more than 4,000 substances, including both substances from human metabolism and substances of other origins.

More than 30,000 mass spectrometric peaks are typically obtained in an MRMS analysis of a urine sample. Each peak represents a urinary substance that has a unique mass in the mass spectrum and that is present in analytically significant amounts. A computer software program can be written using routine methods that locates and measures the location and size of peaks in a mass spectrum. Redundant substances can also be identified through analysis, e.g., with a computer software program written using routine methods. Such an analysis can identify urinary substances that have combined in various chemical combinations during mass spectrometry. On average, about 8 relevant such combinations are found for each original urinary substance.

Mass spectrometric peaks with specific molecular masses present in analytically significant amounts in the urine sample are identified. Specific molecular masses for urinary substances are well known in the art.

The amount of each urinary substance can be calculated quantitatively by summing the mass spectrometric peaks in the spectrum that correspond to the substance and its related combinations. The amounts of urinary substances can be quantitatively correlated with current and future health conditions of interest by analyzing the amounts of substances in the mass spectrum and by calculating the diagnostic coefficient R_{A} and by nonparametric statistics, including the use of log ratios of the amounts. These urinary substances comprise a chemical metabolic profile, wherein the desired quantitative information is contained in the combination of information in a profile of hundreds or even thousands of urinary substances.

Customization of computer software to locate and measure peaks in a mass spectrum and to identify redundant substances, derivatives or combinations is well known in the art. Such custom software can be programmed to perform the following:

1) The mass spectrometer produces a list of amounts of each substance at a particular m/z value. Because it is a very precise mass measurement, the molecular formula can be narrowed down fairly specifically based on mass alone. This becomes easier when compared to a list of possible molecular formulas of known metabolites. So first each peak is tentatively identified.

2) Each peak can include many different isotopes as well as derivatives of the original compound. These peaks occur at specific known offset masses based on the isotope/derivative. Using this information a family of peaks can be assigned to a particular base elemental formula. 3) Combining the amounts of each peak in the family gives the amount of the substance.

Redundant substances, e.g., urinary substances that have combined to form various derivatives or combinations during mass spectrometry, can also be identified using customized computer software that automatically detects these combinations and sums them together. On average, about 8 relevant chemical combinations are found for each original urinary substance. Such derivatives or combinations can be, for example, chemical substitution of single or multiple hydrogen atoms with Na+ or K+ ions in various combinations, or simply differences in mass caused by isotopic variations. Such derivatives or combinations are known in the art.

The amount of each urinary substance can be quantitatively determined by summing the mass spectrometric peaks in the plurality of peaks representing each urinary substance and its chemical derivatives and combinations. This analysis of urinary substances produces a metabolic profile.

The chemical profile obtained from the patient's urine sample can be used for the quantitative measurement of current and future health and the evaluation of means applied to affect that health. Such means can be, for example, drugs, other medical treatments, exercise, nutrition, etc. This is illustrated by the heart attack prediction experimental results in Example 1.

The urinary substances quantitatively correlated with current and future health conditions of interest are determined using software that can be programmed using methods known in the art to identify the various chemical forms of each substance that pass through the mass spectrometer. The quantitative correlation is based on exact mass and chemical principles, using nonparametric statistics such as the well-known Wilcoxon methods, including the use of log ratios of the amounts to evaluate the correlations. These urinary substances quantitatively correlated with current and future health conditions of interest comprise a metabolic profile for the donor. A metabolic profile is a chemical profile wherein the desired quantitative information is contained in the combination of information in a profile of hundreds or even thousands of urinary substances.

According to embodiments of the present method, numbers of metabolites identified and measured in a single analysis, gaining usually small amounts of empirical correlating information from each individual metabolite—the summations of these correlations being used to simultaneously detect and measure many different health profiles that are diagnostically, useful. This technique depends upon the subtle biochemical interactions through which metabolites throughout the metabolism pick up information about one another.

This permits one simple empirical analytical procedure, optimized for those substances that are easy and inexpensive to measure, to gather a wide variety of useful quantitative information characteristic of various aspects of human health.

In each profile, the information from the measured substances in a urine sample is combined mathematically into one number, designated the “diagnostic coefficient,” for each condition of interest, since most uses of this information are one-dimensional as shown in FIG. 2.

First, placing an individual quantitative on a “life-remaining, physiological aging” axis allows him to watch and manipulate his progress along that axis as a function of diet and other adjustable lifestyles. Placing groups of individuals on this “life-remaining, physiological aging” axis allows objective research on such parameters.

Second, placing individuals on a “probability-of-illness” axis is useful in efforts to combat the probability of developing specific illnesses rather than to combat the illnesses after specific physical systems are observed.

Third, if illness is present, placing an individual on a “severity-of-illness” axis is useful in monitoring and optimizing therapy or treatment for the individual's illness.

Fourth, placing an individual on a “quality of life” axis can include any parameter of importance to the individual—even athletic performance, sleep pattern, or just sense of well-being.

Thus, a single inexpensive quantitative metabolic profiling tool allows an individual to be placed on all four of the above-described axes without the need for solving the underlying biochemistry of the condition of interest or using targeted procedures, the expense of which often limits their use.

The metabolic profile can therefore be used for the quantitative measurement of current and future health and the evaluation of means applied to affect that health. This is illustrated by the heart attack prediction experimental results discussed in Example

Calculations

Statistical tests of the discovery and diagnostic reliability of metabolic profiles can be computed in two different and complementary ways. Both these ways use the Wilcoxon method of nonparametric statistics [11]. Much analytical data, by custom and culture, is tested by methods that assume the measurements to be distributed as Gaussian. If the measured values are determined by underlying phenomena that depend upon a significant number of similarly sized, largely independent variables, then the distribution function (range and relative magnitude of the values within the range) of the measurements tends to be Gaussian. For example, human intelligence is found to be Gaussian distributed. If, however, the data is not distributed as a Gaussian or another defined functional form or if it is not known to be so distributed, then “nonparametric” statistics can be used, which do not depend on the distribution function shape. Medical research, including the research associated with the methods disclosed herein, often involves too few measurements to determine the distribution function shapes, so it can be evaluated non-parametrically. Also, in the case of urinary metabolic profiling, research has shown that the measured values are often not distributed as Gaussian [5].

For the first test (of profile presence), the raw mass spectrometric data can be tested for the existence of metabolic profiles for such states, conditions or diseases such as sex, age, prostate cancer, breast cancer, and heart disease, with no data manipulation at all other than normalization to remove systematic variation caused by variable in vivo dilution, primarily from variable water intake by the subjects. Thus, the peak areas of all substances in each urine sample can be divided by a sample-dependent dilution constant derived by four iterations of normalizing [12], using the values of a large number of the peaks in the sample.

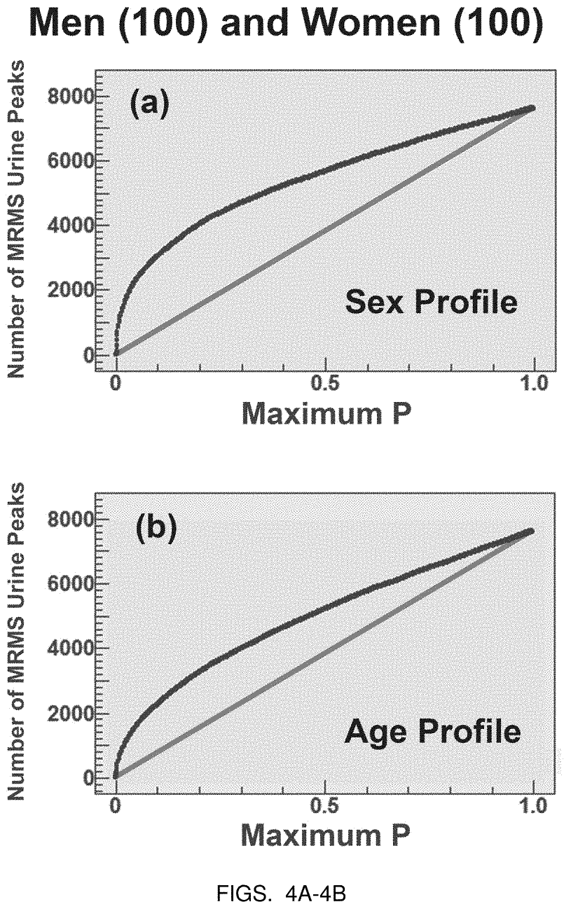

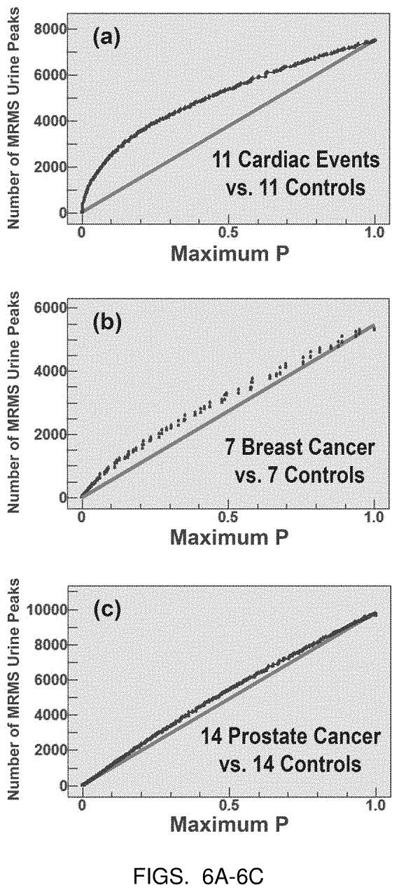

The probabilities of non-correlation (1.0 minus the probabilities of correlation) can then be non-parametrically calculated for each mass spectral peak found in 80% of the spectra of the test subjects and matched controls. Control matching can be, e.g., for sex and age, as appropriate. These probabilities are then arranged in order of increasing magnitude and plotted as cumulative distribution functions, such as those shown in FIGS. 4A-4B and 6A-6C.

For example, if the non-correlation probabilities for peaks in two compared groups for 20 peaks are equal to or lower than P=0.001, that point is plotted; if 35 peak areas lie at or below P=0.002, that point is plotted; and so on for all P value divisions in the comparison. These are the black lines on the graphs. For 5,000 peaks, if there were no correlation and the data were random, 5 peaks would be expected at or below P=0.001; 10 at or below P=0.002; and so on, leading to a linear plot as shown in gray on the graphs. In this example, therefore, 5 peaks are expected at P=0.001 and 20 are found, an excess of 15. This does not reveal which are the random 5 and which are the 15 from a systematic profile, but the excess reveals a profile.

The gray lines shown are theoretically straight but deviate from linearity with finite data sets and experimental noise. So, 20 gray lines are calculated from the measured spectra for 40 control subjects arranged randomly in 20 different paired groups of 20 subjects each. The Gaussian standard deviation, σ, at P=0.1 for such a series of experiments (Gaussian statistics being appropriate for this purpose), can then be computed. Deviation of the black lines from the gray lines can be measured in units of σ, providing values of 5.5σ, 2.2σ, and 0.4σ for the cardiac event, breast cancer, and prostate cancer measurements, respectively.

Thus, when the above-described analysis was conducted in Example 1, there was an estimated greater than 99.99% probability that a predictive cardiac event profile was present, a greater than 95% probability that a predictive breast cancer profile was present (as was discovered for overt breast cancer in the 1970s), and no detected predictive prostate profile at low probabilities of non-correlation, although the overall graph in FIG. 6C of Example 1 revealed an apparent weaker profile for prostate cancer.

For the second test (of profile usefulness), the experimental values can be used in a simple diagnostic procedure to test the diagnostic potential of these profiles. This procedure does not depend on the cumulative distribution functions, but the relative strength of these cumulative distributions will correspond to relative diagnostic power.

Using the method for metabolic profiling, a great many chemical species with unique masses are detectable in urine samples, with more than 100,000 appearing in most of the samples and about 30,000 appearing as unique, reliably quantifiable peaks. On average, each unique substance in these spectra appears at eight specific different masses owing to combinations during analysis with other urinary substances and isotope effects, wherein various elemental isotopic variations appear frequently enough to be detected.

Urine Bank

A “urine bank” can comprise a large collection of many thousands of preserved, e.g., cryogenically frozen, donor urine samples, mass spectra obtained from the many thousands of donor urine samples, and/or the associated health information about the donor providing the urine sample. In an embodiment, the urine bank comprises all three of these components: (1) a large collection of many thousands of cryogenically frozen donor urine samples, (2) the mass spectra obtained from the many thousands of donor urine samples, and (3) the associated health information about the donor providing the urine sample. Health information can be collected periodically, e.g., every 6 months, every year, or any other interval from thousands of individuals from the general public.

Urine samples in the urine bank are donated by, or acquired from, individuals to the urine bank. Urine samples can be donated to the urine bank by donors for the sake of increasing medical knowledge. Patients whose urine is to be analyzed with reference to the information stored in the urine bank can also agree to be donors.

In an embodiment, the urine samples in the urine bank are cryogenically frozen and stored cryogenically at −80° C. Freezing allows for the sample to be preserved unchanged and in certain embodiments, to be thawed at a later time. A sample frozen at −80° C. and thawed decades later will be essentially unchanged, because at that temperature, molecular motion is slowed down so much that changes are extremely slow. Some samples with complex structures can denature due to the freezing/thawing cycle (which is why freezing quickly in liquid nitrogen is used for refreezing and is a standard laboratory technique), however, the urinary substances being identified and measured according to the present method are small molecules that are generally not affected by freezing/thawing cycles.

In another embodiment, the urine sample is dried on a piece of paper (e.g., filter paper) before storage. Such methods for preservation of fluid samples by drying on paper are known in the art. In other embodiments, urine samples can be preserved and stored by any other suitable method known in the art.

As groups of samples accumulate from persons with similar subsequent medical events, samples are quantitatively analyzed by magnetic resonance mass spectrometry (MRMS) as disclosed herein.

Preserved, e.g., cryogenically frozen, urine samples in the urine bank can be analyzed and re-preserved on one or more occasions. Samples can be frozen, then thawed, analyzed, refrozen, and re-thawed at a later time point and analyzed again. Freeze/thaw cycles can be minimized by taking several aliquots of a sample for periodic testing, and by refreezing quickly (e.g., cryogenically) using liquid nitrogen. Re-analysis of samples in the urine bank in the future may yield additional information, especially as new analytic technologies are developed. It is therefore important that the urine samples be preserved in the urine bank be preserved for re-analysis in the future.

MRMS permits the quantitative measurement of more than 800 molecular urinary, substances of human metabolic origin (Table 2) in a single assay. Subjects or patients providing urine samples are analyzed and grouped according to current health and predicted future health. Analyses of metabolic profiles for aging, sex, heart disease, breast cancer, and prostate cancer have proved to be useful for diagnosing and/or predicting the status and progression of these states and conditions. See Examples 1 and 2.

For example, there is a 99.99% probability that a profile predictive of a subsequent cardiac event has been identified, and a 94% and 97% chance, respectively, that profiles predictive of breast or prostate cancer have been identified. Such profiles can be made available at very low cost and can be used in preventive, diagnostic, and therapeutic medicine.

Health information about the donor providing the urine sample can be obtained at the time of donation. This information is stored, along with the metabolic profile of the urine sample, in a computer database. Health information about the subject can be obtained by any means known in the art, such as a questionnaire, a telephone interview or an in-person interview. Health information can be obtained at the time of donation of a urine sample or at any time later—ideally health information is collected at the same time as the sample.

The following examples are offered by way of illustration and not by way of limitation.

### EXAMPLE

### 6.1 Example 1: Metabolic Profiling with Magnetic Resonance Mass Spectrometry and a Human Urine Bank: Profiles for Aging, Sex, Heart Disease, Breast Cancer and Prostate Cancer

Summary

A sample bank has been established at the Oregon Institute of Science and Medicine (OISM, 2251 Dick George Road, Cave Junction, OR 97523) to which 5,000 volunteers are periodically contributing urine specimens and medical histories. Samples are stored at −80° C. (degrees Celsius). As groups of samples accumulate from persons with similar subsequent medical events, samples are quantitatively analyzed by magnetic resonance mass spectrometry (MRMS). MRMS permits the quantitative measurement of more than 800 molecular urinary substances of human metabolic origin (Table 2) in a single analysis. Profiles for aging, sex, heart disease, breast cancer, and prostate cancer have been found and analyzed for diagnostic usefulness. There is a 99.99% probability that a profile predictive of a subsequent cardiac event has been identified, and a 94% and 97% chance, respectively, that profiles predictive of breast or prostate cancer have been identified. Such profiles can be made available at very low cost and can be used in preventive, diagnostic, and therapeutic medicine.

In a set of patients with diagnosed cardiac disease, a diagnostic coefficient greater than a specified threshold was present in 19 of 21 subjects who experienced a cardiac event 4 to 30 months after contributing a urine specimen, but present in only 2 of 21 age and sex-matched controls. Sixteen of the 21 subjects who had experienced a cardiac event 4 to 30 months after contributing a urine specimen had experienced no cardiac event prior to providing the urine sample. In a randomly selected set of 200 undiagnosed healthy subjects (100 men and 100 women), the cardiac event diagnostic coefficient was above the threshold in 28%. About 27% of the U.S. population in this age group is actuarially expected to die from heart disease.

Introduction

This example demonstrates the utility of quantitatively measuring metabolic profiles of samples from a human urine bank. The approach described here can be used as a means of providing early indication of disease, making it ideally compatible with precision or personalized medicine. To facilitate this approach, we used magnetic resonance mass spectrometry (MRMS) for rapid metabolic profiling. MRMS combines the ultra-high performance of Fourier transform ion cyclotron resonance (FTICR) mass spectrometry with the advantages of matrix assisted laser desorption ionization (MALDI) for rapid, straightforward profiling analysis.

MRMS can be configured with any of the various and routine ionization methods such as electrospray (ESI) [2] and MALDI [3, 4]. MALDI was chosen as the ionization method in this example for its simplicity of operation and non-susceptibility to sample carryover, making it suitable as a diagnostic tool. Additionally, MALDI is fast and less vulnerable to the deleterious effects of salt concentration on ionization efficiency.

These characteristics of the MALDI-MRMS combination allowed us to conduct metabolic profiling with greater numbers of substances using a single rapid analysis rather than analyses combined with chromatographic separations or other preparative procedures, which require more time and added expense.

In this example, a single 7-minute MALDI-MRMS run reproducibly resolved more than 100,000 different chemical substances from a 5 μl human urine sample in positive ion mode. Negative ion mode can be used to further increase this inventory of measurable substances. Also, as the inventory of metabolic profiles grows, this method can be refined for analytical turnaround in much less than 7 minutes.

A true cornucopia of information about virtually all-important health conditions that affect human metabolism is detectable by means of a single analysis with this one analytical technique.

We also carried out substantial experimentation with ESI as an alternative to MALDI for this work. ESI can provide more complete ionization; however, it can introduce sample-introduction contamination as well as adsorptive sample substance losses.

This present example demonstrates that MALDI-MRMS provides excellent diagnostic advances in its present form.

Quantitative Metabolic Profiling

Quantitative metabolic profiling originated in a project conducted for 10 years between 1968 and 1978 to test the hypothesis that a single analysis of the amounts of large numbers of metabolites in human body fluids and tissues, followed by computerized pattern recognition, can be used for the quantitative measurement of many aspects of human health [5]. Using mostly chromatographic measurement of between 50 and 150 substances, in primarily human urine with a few experiments on breath and tissues, this project verified this hypothesis by discovering unique profiles for multiple sclerosis, Duchenne dystrophy, Huntington's disease, breast cancer, diet, fasting, sex, diurnal variation, and chemical birth control. With diet control, profiles sufficient to fingerprint single person biochemical individuality were observed, and it was discovered that urinary substances are monomodally, bimodally, and even trimodally distributed in the human population at birth. In addition, profiles characteristic of physiological age were found in fruit flies, mice, and men [5-8].

With the objective of low cost mass screening of people to increase their quality and length of life as illustrated in FIGS. 1A-1B, this 1970s research had three problematic limitations. First, the analytical procedures were slow and expensive. Second, the disease work was carried out on people who were already overtly ill, which introduces systematic variables other than the disease itself. Third, it involved primarily single samples from individuals.

MRMS as utilized herein makes possible very fast and low-cost measurement of thousands of substances in a single assay. Urine samples are acquired from thousands of individuals of all ages and conditions of health. Furthermore, multiple samples taken over an extended time period can allow individuals to serve as their own controls and markedly, enhanced the precision of the metabolic profiles.

A urine sample bank (“urine bank”) was created at the Oregon Institute of Science and Medicine with urine samples and medical data being collected periodically from 5,000 volunteers. The urine samples were cryogenically frozen and stored at −80° C. The research described in this example that used this database (which includes MRMS data on the urine samples and associated health information about donors of the urine samples) is conceptually different from the vast worldwide effort begun by biochemists a century ago to ultimately and thoroughly understand human metabolism and, along the way, to identify biochemical markers or, now, groups of markers that carry information useful for specific medical purposes [9, 10].

We sought instead to measure large numbers of metabolites in a single analysis, gaining usually small amounts of empirical correlating information from each individual metabolite—the summations of these correlations being used to simultaneously detect and measure many different health profiles that are diagnostically useful. This technique depends upon the subtle biochemical interactions through which metabolites throughout the metabolism pick up information about one another.

This permits one simple empirical analytical procedure, optimized for those substances that are easy and inexpensive to measure, to gather a wide variety of useful quantitative information characteristic of various aspects of human health.

In each profile, the information from the measured substances in a urine sample is combined mathematically into one number, designated the “diagnostic coefficient,” for each condition of interest, since most uses of this information are one-dimensional as shown in FIG. 2.

First, placing an individual quantitatively on a “life-remaining, physiological aging” axis allows him to watch and manipulate his progress along that axis as a function of diet and other adjustable lifestyles. Placing groups of individuals on this “life-remaining, physiological aging” axis allows objective research on such parameters.

Second, placing individuals on a “probability-of-illness” axis is useful in efforts to combat the probability of developing specific illnesses rather than waiting until symptomatic illness is causing the patient to suffer.

Third, if illness is present, placing an individual on a “severity-of-illness” axis is useful in monitoring and optimizing therapy or treatment for the individual's illness.

Fourth, placing an individual on a “quality of life” axis can include any parameter of importance to the individual—even athletic performance, sleep pattern, or just sense of well-being.

Thus, a single inexpensive quantitative metabolic profiling tool allows an individual to be placed on all four of the above-described axes without the need for solving the underlying biochemistry of the condition of interest or using targeted procedures, the expense of which often limits their use.

In 1968, Linus Pauling and Arthur Robinson were searching for ways in which to determine optimum nutritional intakes of essential nutrients in individuals and populations. They, needed to make graphs of health as a function of intake of vitamins and other nutritional substances but lacked a quantitative means of measuring biochemical health. Quantitative metabolic profiling was devised as a possible solution.

While biochemistry is expected to ultimately provide learned answers to these questions, the goal was then and is now to provide empirical information at very low cost to improve the lives of people living now and prior to the ultimate maturation of biochemical knowledge.

The human survival curve includes, as shown in FIGS. 1A-1B, a large percentage of people who experience suffering and death at ages far shorter than the intrinsic human life span. The methods disclosed herein provide the ability to significantly improve the human survival curve. Technological advance in mass spectrometry makes possible not only eventual detailed understanding of human metabolism, but also empirical methods to markedly and significantly reduce this early suffering and death. We describe, herein, a method for using mass spectrometry to measure to quantitatively measure health which will allow early detection and monitoring of diseases and other conditions.

Materials and Methods

Urine Bank

A total of 8,500 interested volunteers in southern and central Oregon were located by direct mail. After expected initial losses, 5,000 volunteers actively participated in this project, with an attrition and necessary replacement rate of about 5% per year over 5 years.

Periodic urine samples and current self-reported medical information were collected from the volunteers. Each sampling consisted of two approximately 1.5 ml (1,500 μl) samples of urine placed in 1.8 ml Nunc Cryotube vials.

In the initial stages of the project, these samples were mailed in ambient temperature United States Postal Service (USPS) approved mailers to the urine bank, where they were cataloged and stored at −80° C. Later, both mailed samples and door-to-door samples that were frozen immediately were collected.

There were partial losses of some substances during the ambient temperature mailing, hut, with thousands of substances from which to choose, these losses were moderate. Door-to-door collection of samples was about twice as expensive as mailing in samples. Collecting mailed-in samples was also more compatible with the goal of keeping costs low, thereby enabling as many people as possible to afford and benefit from this method.

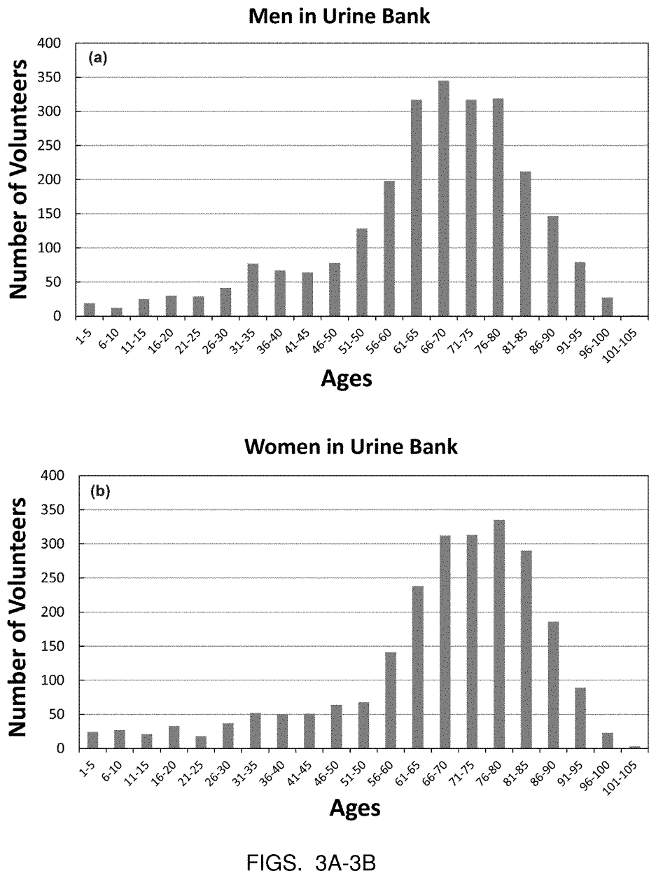

Self-selection led to an older age distribution of volunteers as shown in FIGS. 3A-3B, so they had expected disease incidences about three times greater than a linear age distribution of ordinary Americans.

The urine bank storage is maintained in military grade −80° C. freezers with three-fold power backup. The two samples per volunteer permit storage in two locations.

Mass Spectrometry

Mass spectral analysis was performed in an unmodified Bruker 7T-SolariX XR ICR FINIS (Bruker Daltonics, tuned to the 100 to 1,000 m/z mass range. The MALDI (matrix assisted laser desorption ionization) source was operated at 50% laser power. The MALDI plate was a Protea Biosciences Redichip, nanopost type with no chemical matrix.

Urine sample dilutions were determined by spectrophotometry over 350-360 nanometers in a Molecular Devices SpectraMax M2 spectrophotometer, with the concentrations then normalized by adding between 0 and 50 μl of VWR Aristar Ultra-pure water to a 5 μl urine sample. This adjusted the urine concentrations approximately with one another. A total of 4 μl of each diluted sample was carefully applied to the MALDI plate to completely cover one circular nanopost array and dried before analysis.

A total of 200 FTICR transients were averaged together, with each 1.0-second transient generated by a 500-shot pulsed laser directed onto a unique position on the plate as selected by means of Bruker automation. The sample cycle time was seven minutes.

Ions from the 500 laser shots accumulate in the MALDI source and enter the ICR cell as one group, the measurement of which produces one transient analytical image. The average of 200 such transients is converted into the mass spectrum by Fourier transform.

With 200 transients and an estimated 1 million molecules accommodated by the ICR cell, an estimated 200 million molecules varying in amounts over three orders of magnitude can be measured. These are composed of more than 100,000 molecular components, with about 30,000 making up most of the total. So, most of the individual chemical species are present in very small amounts.

Therefore, the analytical system is preferably very clean. The use of laser desorption ionization overcomes contamination of the sample during introduction, but the MALDI chemical matrices commercially available to us were unacceptably contaminated with impurities. We used nanopost-type plates, which were sufficiently clean. Similarly, ordinary desalting procedures are sources of sample contamination and sample loss at these low concentrations of urinary substances, so desalting was omitted, which also simplifies the procedure.

MRMS technology is a preferred choice for this application because of its evident superior capabilities as illustrated herein and because of marked improvements in MRMS technology that are continuing to be made.

The quantitative noise in the measurements reported herein is manageable. The high sample quality and especially the large number of experimental parameters utilized have partially overcome noise in these experiments. Suppression of noise, which is continuing to be improved by advances in MRMS technology, and the maturation of the OISM urine sample bank over time, will provide even more remarkable profiling capability.

Subjects Used in the Analyses

For the age and sex analyses, 1.00 men and 100 women spanning the age range and distribution shown in FIGS. 3A-3B were drawn from volunteers who reported good health. For the volunteers who reported a diagnosis of breast or prostate cancer, we analyzed samples given before the cancer was otherwise diagnosed—all compared with individually age and sex-matched controls.

The cardiac event testing was conducted twice. The first trial was with 11 cardiac-event subjects and 11 age and sex-matched controls, with five of the subjects having experienced heart problems prior to providing the urine sample and six having not experienced a prior heart problem. After this was done, we received reports from 10 additional volunteers (or their survivors) that they had experienced their first known cardiac event. We then analyzed the samples provided by the 16 volunteers who had not reported cardiac symptoms prior to providing a urine sample. Of the 21 subjects with cardiac events in the two trials, “heart attacks” were reported for 14 subjects, “congestive heart failure” for 4, and “heart failure” for 3.

Calculations

Statistical tests of the discovery and diagnostic reliability of the metabolic profiles reported here were computed in two different and complementary ways. Both these ways use the Wilcoxon method of nonparametric statistics [11]. Much analytical data, by custom and culture, is tested by methods that assume the measurements to be distributed as Gaussian. If the measured values are determined by underlying phenomena that depend upon a significant number of similarly sized, largely independent variables, then the distribution function (range and relative magnitude of the values within the range) of the measurements tends to be Gaussian. For example, human intelligence is found to be Gaussian distributed. If, however, the data is not distributed as a Gaussian or another defined functional form or if it is not known to be so distributed, then “nonparametric” statistics can be used for analysis, which do not depend on the distribution function shape. Medical research, including that reported herein, often involves too few measurements to determine the distribution function shapes, so it can be evaluated non-parametrically. Also, in the case of urinary metabolic profiling, research has shown that the measured values are often not distributed as Gaussian [5].

For the first test (of profile presence), the raw mass spectrometric data herein were tested for the existence of metabolic profiles for sex, age, prostate cancer, breast cancer, and heart disease, with no data manipulation at all other than normalization to remove systematic variation caused by variable in vivo dilution, primarily from variable water intake by the subjects. Thus, the peak areas of all substances in each urine sample were divided by a sample-dependent dilution constant derived by four iterations of normalizing [12], using the values of a large number of the peaks in the sample.

The probabilities of non-correlation (1.0 minus the probabilities of correlation) were then non-parametrically calculated for each mass spectral peak found in 80% of the spectra of the test subjects and matched controls. Control matching was primarily for sex and age, as appropriate. These probabilities were arranged in order of increasing magnitude and plotted as cumulative distribution functions as shown in FIGS. 4A-4B and 6A-6C.

So, for example, if the non-correlation probabilities for peaks in two compared groups for 20 peaks are equal to or lower than P=0.001, that point is plotted; if 35 peak areas lie at or below P=0.002, that point is plotted; and so on for all P value divisions in the comparison. These are the black lines on the graphs. For 5,000 peaks, if there were no correlation and the data were random, 5 peaks would be expected at or below P=0.001; 10 at or below P=0.002; and so on, leading to a linear plot as shown in gray on the graphs. In this example, therefore, 5 peaks are expected at P=0.001 and 20 are found, an excess of 15. This does not reveal which are the random 5 and which are the 15 from a systematic profile, but the excess reveals a profile.

The gray lines shown are theoretically straight but deviate from linearity with finite data sets and experimental noise. So, we calculated 20 gray lines from the measured spectra for 40 control subjects arranged randomly in 20 different paired groups of 20 subjects each. The Gaussian standard deviation, σ, at P=0.1 for this series of experiments (Gaussian statistics being appropriate for this purpose), was computed. Deviation of the black lines from the gray lines was thus measured in units of σ, providing values of 5.5 σ, 2.2σ, and 0.4σ for the cardiac event, breast cancer, and prostate cancer measurements, respectively. So, there is an estimated greater than 99.99% probability that a predictive cardiac event profile is present, a greater than 95% probability that a predictive breast cancer profile is present (as was discovered for overt breast cancer in the 1970s), and no detected predictive prostate profile at low probabilities of non-correlation, although the overall graph reveals an apparent weaker profile for prostate cancer.

For the second test (of profile usefulness), the experimental values were used in a simple diagnostic procedure to test the diagnostic potential of these profiles. This procedure does not depend on the cumulative distribution functions, but it is to be expected that the relative strength of these cumulative distributions would correspond to relative diagnostic power, as it does.

Using the method for metabolic profiling, a great many chemical species with unique masses are detectable in these urine samples, with more than 100,000 appearing in most of the samples and about 30,000 appearing as unique, reliably quantifiable peaks. On average, each unique substance in these spectra appears at eight specific different masses due to combinations during analysis with other urinary substances and isotope effects, wherein various elemental isotopic variations appear frequently enough to be detected.

A recent review of urine composition [13] lists 2,700 unique chemical substances that have been detected in human urine in the mass range of our experiments, with 917 of those listed believed to be endogenous products of human metabolism. These could include both human and bacterial products and byproducts. We tentatively identified 2,300 of these in our spectra based upon their masses being within 2.5 parts per million of the exact theoretical masses in the 2,700. We verified 833 (Table 2) of the 917 by means of observed masses of multiple adduct forms and isotopes and found that about 700 met the criteria of appearing in 80% of the samples in each profiling experiment. We added all detected amounts of different mass forms of each of the 833 together to obtain the total amount of each unique substance used in the diagnostic calculations.

The molecular identities of these 833 substances have been tentatively determined by exact mass and are listed in Table 2. This mass measurement provides elemental formulas, not structural formulas. The molecular identities have been enhanced by structural information regarding urine composition compiled from other sources [13], but it is to be expected that some of these assigned molecular identities may be incorrect.

Table 2 below lists 833 possible endogenous matched molecules. Peaks were matched to within 2.5 ppm of the Monoisotopic mass shown. The chemical formula listed corresponds to this mass and is a possible endogenous molecule in human urine, however, in some cases other formulas could fit the mass. The chemical names given are taken from the Human Urine Metabolome database as published in reference 6. In most cases, one of these exact identifications will be correct; however, they are indistinguishable from other possible isomers.

We did not filter these chemical formulas for ionization probabilities in MALDI.

TABLE 2
The 833 urinary chemical substances found and utilized as described herein.

| Identified | | |
|---|---|---|
| Monoisotopic Mass | Possible Chemical | |
| (2.5 ppm) | Formula | Possible Endogenous Molecules |
| 31.0422 | CH5N | Methylamine |
| 32.0374 | H4N2 | Hydrazine |
| 33.0215 | H3NO | Hydroxylamine |
| 45.0578 | C2H7N | Dimethylamine; Ethylamine |
| 58.0419 | C3H6O | Acetone |
| 59.0483 | CH5N3 | Guanidine |
| 60.0211 | C2H4O2 | Acetic acid |
| 60.0324 | CH4N2O | Urea |
| 60.0575 | C3H8O | Isopropyl alcohol |
| 60.0687 | C2H8N2 | 1,2-Ethanediamine |
| 61.0528 | C2H7NO | Ethanolamine |
| 68.0262 | C4H4O | Furan |
| 72.0211 | C3H4O2 | Pyruvaldehyde; Malondialdehyde |
| 72.0575 | C4H8O | Butanone |
| 73.0528 | C3H7NO | N,N-Dimethylformamide; Aminoacetone |
| 73.0640 | C2H7N3 | Methylguanidine |
| 74.0004 | C2H2O3 | Glyoxylic acid |
| 74.0368 | C3H6O2 | Propionic acid; Hydroxyacetone |
| 74.0844 | C3H10N2 | 1,3-Diaminopropane; 1,2 Diaminopropane |
| 75.0320 | C2H5NO2 | Glycine; Acetohydroxamic Acid |
| 75.0684 | C3H9NO | Trimethylamine N-oxide |
| 76.0160 | C2H4O3 | Glycolic acid |
| 77.0299 | C2H7NS | Cysteamine |
| 77.9872 | C2H3ClO | Chloroacetaldehyde |
| 82.0419 | C5H6O | 2-Methylfuran |
| 88.0160 | C3H4O3 | Pyruvic acid |
| 88.0524 | C4H8O2 | Butyric acid; Isobutyric acid; Acetoin; Ethyl |
| | | acetate |
| 88.1000 | C4H12N2 | Putrescine |
| 89.0477 | C3H7NO2 | Beta-Alanine; L-Alanine; Sarcosine |
| 89.9953 | C2H2O4 | Oxalic acid |
| 90.0317 | C3H6O3 | L-Lactic acid; Hydroxypropionic acid; |
| | | Glyceraldehyde; D-Lactic acid; |
| | | Dihydroxyacetone |
| 92.0473 | C3H8O3 | Glycerol |
| 93.0578 | C6H7N | Aniline |
| 94.0089 | C2H6O2S | Dimethyl sulfone |
| 97.9769 | H3O4P | Phosphoric acid |
| 98.0368 | C5H6O2 | 2-Furanmethanol |
| 100.0524 | C5H8O2 | 4-Pentenoic acid; Dihydro-5-methyl-2(3H)- |
| | | furanone |
| 100.0888 | C6H12O | 3-Hexanone; Methyl isobutyl ketone; 2- |
| | | Oxohexane; Hexanal |
| 102.0317 | C4H6O3 | 2-Ketobutyric acid; Acetoacetic acid; Succinic |
| | | acid semialdehyde |
| 102.0681 | C5H10O2 | Isovaleric acid; Valeric acid; Ethylmethylacetic |
| | | acid; (S)-2-Methylbutanoic acid; 1-Hydroxy-2- |
| | | pentanone |
| 102.1157 | C5H14N2 | Cadaverine |
| 103.0633 | C4H9NO2 | Dimethylglycine; Gamma-Aminobutyric acid; L- |
| | | Alpha-aminobutyric acid; D-Alpha-aminobutyric |
| | | acid; 2-Aminoisobutyric acid; (S)-b- |
| | | aminoisobutyric acid; 3-Aminoisobutanoic acid |
| 104.0110 | C3H4O4 | Maionic acid; Hydroxypyruvic acid |
| 104.0473 | C4H8O3 | 2-Hydroxybutyric acid; (R)-3-Hydroxybutyric |
| | | acid; (S)-3-Hydroxyisobutyric acid; (R)-3- |
| | | Hydroxyisobutyric acid; 3-Hydroxybutyric acid; |
| | | (S)-3-Hydroxybutyric acid; 4-Hydroxybutyric |
| | | acid; Alpha-Hydroxyisobutyric acid |
| 104.0586 | C3H8N2O2 | 2,3-Diaminopropionic acid |
| 105.0426 | C3H7NO3 | L-Serine; D-Serine |
| 106.0266 | C3H6O4 | Glyceric acid; L-Glyceric acid |
| 108.0211 | C6H4O2 | Quinone |
| 108.0575 | C7H8O | p-Cresol; m-Cresol; o-Cresol; Anisole |
| 109.0197 | C2H7NO2S | Hypotaurine |
| 109.0528 | C6H7NO | 4-Aminophenol |
| 110.0368 | C6H6O2 | Pyrocatechol; Hydroquinone |
| 111.0320 | C5H5NO2 | Pyrrole-2-carboxylic acid |
| 111.0433 | C4H5N3O | Cytosine |
| 111.0796 | C5H9N3 | Histamine; Betazole |
| 112.0160 | C5H4O3 | 2-Furoic acid |
| 112.0273 | C4H4N2O2 | Uracil; 4-Carboxypyrazole |
| 113.0589 | C4H7N3O | Creatinine |
| 113.9929 | C2HF3O2 | Trifluoroacetic acid |
| 114.0429 | C4H6N2O2 | Dihydrouracil; N-Methylhydantoin |
| 114.1045 | C7H14O | 2-Heptanone; 4-Heptanone; 5-Methyl-2- |
| | | hexanone; 1-Methylcyclohexanol |
| 115.0633 | C5H9NO2 | L-Proline; D-Proline |
| 116.0110 | C4H4O4 | Fumaric acid; Maleic acid |
| 116.0473 | C5H8O3 | Alpha-ketoisovaleric acid; Levulinic acid; 2- |
| | | Oxovaleric acid; 2-Methylacetoacetic acid |
| 116.0837 | C6H12O2 | Caproic acid; Isocaproic acid |
| 117.0426 | C4H7NO3 | Acetylglycine |
| 117.0538 | C3H7N3O2 | Guanidoacetic acid |
| 117.0578 | C8H7N | Indole |
| 117.0790 | C5H11NO2 | Betaine; L-Valine; 5-Aminopentanoic acid; |
| | | Norvaline; Amyl Nitrite |
| 118.0266 | C4H6O4 | Methylmalonic acid; Succinic acid |
| 118.0630 | C5H10O3 | 2-Methyl-3-hydroxybutyric acid; 2- |
| | | Ethylhydracrylic acid; 2-Hydroxy-3- |
| | | methylbutyric acid; 3-Hydroxyvaleric acid; 3- |
| | | Hydroxyisovaleric acid; 2-Hydroxyvaleric acid; |
| | | 2-Hydroxy-2-methylbutyric acid; 4- |
| | | Hydroxyisovaleric acid |
| 118.0742 | C4H10N2O2 | 2,4-Diaminobutyric acid; L-2,4-diaminobutyric |
| | | acid |
| 119.0219 | C3H5NO4 | Aminomalonic acid |
| 119.0582 | C4H9NO3 | L-Threonine; L-Homoserine; L-Allothreonine; |
| | | Hydroxyethyl glycine |
| 120.0245 | C4H8O2S | 3-Methylthiopropionic acid |
| 120.0423 | C4H8O4 | (S)-3,4-Dihydroxybutyric acid; 2,4- |
| | | Dihydroxybutanoic acid; 4-Deoxyerythronic acid; |
| | | 4-Deoxythreonic acid; A,b-Dihydroxyisobutyric |
| | | acid |
| 120.0575 | C8H8O | 2,3-Dihydrobenzofuran |
| 121.0197 | C3H7NO2S | L-Cysteine |
| 121.0891 | C8H11N | 1-Phenylethylamine; Phenylethylamine; 2,6- |
| | | Dimethylaniline |
| 122.0480 | C6H6N2O | Niacinamide |
| 122.0579 | C4H10O4 | Erythritol; D-Threitol |
| 122.0732 | C8H10O | 4-Ethylphenol |
| 123.0320 | C6H5NO2 | Nicotinic acid; Picolinic acid; Isonicotinic acid |
| 124.0524 | C7H8O2 | 4-Methylcatechol; Guaiacol |
| 125.0147 | C2H7NO3S | Taurine |
| 125.0953 | C6H11N3 | 1-Methylhistamine; 3-Methylhistamine |
| 125.9987 | C2H6O4S | 2-Hydroxyethanesulfonate |
| 126.0317 | C6H6O3 | 1,2,3-Trihydroxybenzene; 5-Methylfuran-2- |
| | | carboxylic acid |
| 126.0429 | C5H6N2O2 | Thymine; Imidazoleacetic acid |
| 126.0542 | C4H6N4O | 5-Aminoimidazole-4-carboxamide |
| 126.1045 | C8H14O | (E)-2-octenal |
| 128.0473 | C6H8O3 | 3-Hydroxy-4,5-dimethyl-2(5H)-furanone |
| 128.0586 | C5H8N2O2 | Dihydrothymine |
| 129.0426 | C5H7NO3 | Pyroglutamic acid; dimethadione |
| 129.0790 | C6H11NO2 | Pipecolic acid; Vigabatrin |
| 130.0266 | C5H6O4 | Glutaconic acid; Citraconic acid; 2- |
| | | Hydroxyglutaric acid lactone; 2-Pentendioate |
| 130.0630 | C6H10O3 | 2-Methyl-3-ketovaleric acid; 3-Methyl-2- |
| | | oxovaleric acid; Ketoleucine; Mevalonolactone |
| 130.1106 | C6H14N2O | N-Acetylputrescine |
| 130.1218 | C5H14N4 | Agmatine |
| 130.1358 | C8H18O | Octanol; 2-Ethyl-4-methyl-1-pentanol |
| 131.0582 | C5H9NO3 | 4-Hydroxyproline; N-Acetyl-L-alanine; |
| | | Propionylglycine; 5-Aminolevulinic acid; 4- |
| | | Hydroxy-L-proline |
| 131.0695 | C4H9N3O2 | Creatine |
| 131.0946 | C6H13NO2 | L-Isoleucine; L-Alloisoleucine; L-Leucine; L- |
| | | Norleucine; N-(2-Hydroxyethyl)-morpholine |
| 132.0059 | C4H4O5 | Oxalacetic acid |
| 132.0423 | C5H8O4 | Ethylmalonic acid; Glutaric acid; Methylsuccinic |
| | | acid |
| 132.0535 | C4H8N2O3 | Ureidopropionic acid; L-Asparagine; Glycyl- |
| | | glycine |
| 132.0786 | C6H12O3 | 2-Hydroxy-3-methylpentanoic acid; 5- |
| | | Hydroxyhexanoic acid; Leucinic acid; |
| | | Hydroxyisocaproic acid; 2-Hydroxycaproic acid; |
| | | Threo-3-Hydroxy-2-methylbutyric acid |
| 132.0899 | C5H12N2O2 | Ornithine |
| 132.1025 | C6H14NO2 | 1-Nitrohexane |
| 133.0375 | C4H7NO4 | L-Aspartic acid; D-Aspartic acid; Iminodiacetic |
| | | acid |
| 134.0215 | C4H6O5 | L-Malic acid; Malic acid |
| 135.0354 | C4H9NO2S | Homocysteine; Methylcysteine |
| 135.0545 | C5H5N5 | Adenine |
| 136.0372 | C4H8O5 | Erythronic acid; Threonic acid |
| 136.0385 | C5H4N4O | Hypoxanthine; Allopurinol |
| 136.0524 | C8H8O2 | Phenylacetic acid; 2-Methylbenzoic acid |
| 136.0637 | C7H8N2O | N-Methylnicotinamide |
| 136.0736 | C5H12O4 | 2-Methylerythritol |
| 136.9584 | C2H6AsO2 | Dimethylarsinate |
| 137.0477 | C7H7NO2 | Trigonelline; 2-Aminobenzoic acid; p- |
| | | Aminobenzoic acid; m-Aminobenzoic acid; |
| | | Salicylamide; 2-Pyridylacetic acid |
| 137.0715 | C7H9N2O | 1-Methylnicotinamide; Pralidoxime |
| 137.0841 | C8H11NO | Tyramine; m-Tyramine; 4-Hydroxy-2,6- |
| | | dimethylaniline |
| 138.0317 | C7H6O3 | 4-Hydroxybenzoic acid; Salicylic acid; 3- |
| | | Hydroxybenzoic acid; 3,4- |
| | | Dihydroxybenzaldehyde |
| 138.0429 | C6H6N2O2 | Urocanic acid |
| 138.0681 | C8H10O2 | Tyrosol |
| 138.1045 | C9H14O | 2-Pentylfuran |
| 139.0269 | C6H5NO3 | 4-Nitrophenol; 6-Hydroxynicotinic acid; 3- |
| | | Hydroxypicolinic acid |
| 140.0586 | C6H8N2O2 | Methylimidazoleacetic acid; Pi- |
| | | Methylimidazoleacetic acid |
| 140.1201 | C9H16O | 2-Nonenal |
| 141.0191 | C2H8NO4P | O-Phosphoethanolamine |
| 142.0266 | C6H6O4 | trans-trans-Muconic acid; Sumiki's acid; 2,3- |
| | | Methylenesuccinic acid |
| 142.0378 | C5H6N2O3 | 5-Hydroxymethyluracil |
| 142.0994 | C8H14O2 | 4-ene-Valproic acid; 2-ene-Valproic acid; (3Z)-2- |
| | | Propylpent-3-enoic acid; (3E)-2-Propylpent-3- |
| | | enoic acid |
| 142.1358 | C9H18O | 2-Methyl-4-heptanone; 2-Nonanone |
| 143.0946 | C7H13NO2 | Proline betaine |
| 144.0423 | C6H8O4 | 3-Methylglutaconic acid; (E)-2-Methylglutaconic |
| | | acid |
| 144.0575 | C10H8O | 1-Naphthol; 2-Naphthol |
| 144.0786 | C7H12O3 | 4-Hydroxycyclohexylcarboxylic acid |
| 144.1150 | C8H16O2 | Caprylic acid |
| 144.1263 | C7H16N2O | N-Acetylcadaverine |
| 145.0739 | C6H11NO3 | Isobutyrylglycine; N-Butyrylglycine; 4- |
| | | Acetamidobutanoic acid; Methyl |
| | | aminolevulinate; N-(2-Carboxymethyl)- |
| | | morpholine |
| 145.0851 | C5H11N3O2 | 4-Guanidinobutanoic acid |
| 145.1579 | C7H19N3 | Spermidine |
| 146.0215 | C5H6O5 | Oxoglutaric acid; 3-Oxoglutaric acid |
| 146.0579 | C6H10O4 | 2-Methylglutaric acid; Adipic acid; |
| | | Methylglutaric acid; Monomethyl glutaric acid; |
| | | Solerol |
| 146.0691 | C5H10N2O3 | L-Glutamine; Ureidoisobutyric acid |
| 146.1181 | C7H16NO2 | 4-Trimethylammoniobutanoic acid; 1- |
| | | Nitroheptane |
| 147.0532 | C5H9NO4 | L-Glutamic acid; N-Acetylserine; O- |
| | | Acetylserine; D-Glutamic acid |
| 148.0194 | C5H8O3S | 2-Oxo-4-methylthiobutanoic acid |
| 148.0372 | C5H8O5 | Citramalic acid; 3-Hydroxyglutaric acid; D-2- |
| | | Hydroxyglutaric acid; L-2-Hydroxyglutaric acid; |
| | | Ribonolactone; 2-Hydroxyglutarate |
| 148.0524 | C9H8O2 | Cinnamic acid |
| 148.0736 | C6H12O4 | Mevalonic acid |
| 149.0701 | C6H7N5 | 1-Methyladenine; 3-Methyladenine |
| 149.9987 | C4H6O4S | Thiodiacetic acid |
| 150.0317 | C8H6O3 | Phenylglyoxylic acid |
| 150.0528 | C5H10O5 | D-Xylose; D-Ribose; L-Arabinose; L-Threo-2- |
| | | pentulose; D-Xylulose; Arabinofuranose; 2- |
| | | Deoxypentonic acid |
| 150.0681 | C9H10O2 | Hydrocinnamic acid; 4-Ethylbenzoic acid; 2- |
| | | Phenylpropionate; 2-Methoxy-4-vinylphenol |
| 150.0793 | C8H10N2O | 6-Methylnicotinamide |
| 150.1045 | C10H14O | Thymol; (+)-(S)-Carvone; (R)-Carvone; 5- |
| | | Isopropyl-2-methylphenol; Carvone |
| 151.0494 | C5H5N5O | Guanine; 2-Hydroxyadenine |
| 151.0633 | C8H9NO2 | Acetaminophen; 2-Phenylglycine; Dopamine |
| | | quinone; 2-Amino-3-methylbenzoate |
| 152.0334 | C5H4N4O2 | Xanthine; Oxypurinol |
| 152.0473 | C8H8O3 | p-Hydroxyphenylacetic acid; 3- |
| | | Hydroxyphenylacetic acid; Ortho- |
| | | Hydroxyphenylacetic acid; Mandelic acid; 3- |
| | | Cresotinic acid; 4-Hydroxy-3-methylbenzoic |
| | | acid; Vanillin; Methylparaben; 2- |
| | | Methoxybenzoic acid; 3-Methoxybenzoic acid; |
| | | Methyl 2-hydroxybenzoate |
| 152.0586 | C7H8N2O2 | N1-Methyl-2-pyridone-5-carboxamide; N1- |
| | | Methyl-4-pyridone-3-carboxamide |
| 152.0685 | C5H12O5 | Ribitol; D-Arabitol; L-Arabitol; D-Xylitol |
| 152.1201 | C10H16O | Piperitone; beta-Cyclocitral; 4-(1-Methylethyl)-1- |
| | | cyclohexene-4-carboxaldehyde |
| 153.0426 | C7H7NO3 | 3-Hydroxyanthranilic acid; 3-Aminosalicylic |
| | | acid; Aminosalicylic Acid; Mesalazine; 6- |
| | | Methoxy-pyridine-3-carboxylic acid |
| 153.0651 | C5H7N5O | FAPy-adenine |
| 153.0790 | C8H11NO2 | Dopamine; p-Octopamine |
| 154.0266 | C7H6O4 | Gentisic acid; 2-Pyrocatechuic acid; |
| | | Protocatechuic acid; 2,6-Dihydroxybenzoic acid; |
| | | 3,5-Dihydroxybenzoic acid; 2,4- |
| | | Dihydroxybenzoic acid |
| 154.0395 | C4H11O4P | Diethylphosphate |
| 155.9978 | C7H5ClO2 | m-Chlorobenzoic acid |
| 156.0059 | C6H4O5 | 2,5-Furandicarboxylic acid |
| 156.0171 | C5H4N2O4 | Orotic acid |
| 156.0535 | C6H8N2O3 | Imidazolelactic acid |
| 156.1150 | C9H16O2 | 4-Hydroxynonenal |
| 156.1514 | C10H20O | Menthol; Decanal; (E)-3-decen-1-ol; (−)- |
| | | Neoisomenthol |
| 157.0739 | C7H11NO3 | 3-Methylcrotonylglycine; Tiglylglycine; |
| | | Paramethadione |
| 158.0440 | C4H6N4O3 | Allantoin |
| 158.0579 | C7H10O4 | Succinylacetone |
| 158.0943 | C8H14O3 | cis-4-Hydroxycyclohexylacetic acid; trans-4- |
| | | Hydroxycyclohexylacetic acid; 2-n-Propyl-4- |
| | | oxopentanoic acid; 3-Oxovalproic acid |
| 159.0895 | C7H13NO3 | 2-Methylbutyrylglycine; Isovalerylglycine |
| 159.1259 | C8H17NO2 | DL-2-Aminooctanoic acid; Pregabalin |
| 160.0372 | C6H8O5 | Oxoadipic acid; 3-Methyl-3- |
| | | hydroxypentanedioate |
| 160.0736 | C7H12O4 | 3-Methyladipic acid; Pimelic acid; 2- |
| | | Ethylglutaric acid |
| 160.1000 | C10H12N2 | Tryptamine; Tolazoline |
| 160.1099 | C8H16O3 | 7-Hydroxyoctanoic acid; 5-Hydroxyvalproic |
| | | acid; 3-Hydroxyvalproic acid; 4-Hydroxyvalproic |
| | | acid |
| 160.1212 | C7H16N2O2 | N(6)-Methyllysine |
| 161.0324 | C5H7NO5 | A-Ketoglutaric acid oxime |
| 161.0477 | C9H7NO2 | 2-Indolecarboxylic acid |
| 161.0688 | C6H11NO4 | Aminoadipic acid |
| 161.1052 | C7H15NO3 | L-Carnitine |
| 161.1079 | C10H13N2 | Nicotine imine |
| 161.1290 | C7H17N2O2 | Putreanine; Bethanechol |
| 162.0292 | C7H5F3O | para-Trifluoromethylphenol |
| 162.0317 | C9H6O3 | Umbelliferone |
| 162.0528 | C6H10O5 | 2-Hydroxyadipic acid; 3-Hydroxyadipic acid; 3- |
| | | Hydroxymethylglutaric acid; Levoglucosan; 2- |
| | | Hydroxy-2-ethylsuccinic acid |
| 162.0793 | C9H10N2O | Norcotinine |
| 162.1004 | C6H14N2O3 | 5-Hydroxylysine |
| 163.0303 | C5H9NO3S | Acetylcysteine |
| 164.0473 | C9H8O3 | Phenylpyruvic acid; m-Coumaric acid; 4- |
| | | Hydroxycinnamic acid; Coumaric acid |
| 164.0685 | C6H12O5 | L-Fucose |
| 165.0460 | C5H11NO3S | Methionine sulfoxide |
| 165.0651 | C6H7N5O | N2-Methylguanine |
| 166.0266 | C8H6O4 | Phthalic acid; Terephthalic acid |
| 166.0491 | C6H6N4O2 | 3-Methylxanthine; 7-Methylxanthine; 1- |
| | | Methylxanthine |
| 166.0630 | C9H10O3 | 3-(3-Hydroxyphenyl)propanoic acid; Phenyllactic |
| | | acid; 4-Methoxyphenylacetic acid; |
| | | Desaminotyrosine; 3,4-Dihydroxyphenylacetone; |
| | | 4-Hydroxyphenyl-2-propionic acid; 3- |
| | | Methoxyphenylacetic acid |
| 166.0994 | C10H14O2 | Perillic acid |
| 167.0219 | C7H5NO4 | Quinolinic acid |
| 167.0946 | C9H13NO2 | 3-Methoxytyramine; Phenylephrine; p- |
| | | Synephrine; Metaraminol; Ethinamate; 4- |
| | | Hydroxynorephedrine; a-Methyldopamine |
| 168.0283 | C5H4N4O3 | Uric acid |
| 168.0423 | C8H8O4 | Homogentisic acid; Vanillic acid; 3- |
| | | Hydroxymandelic acid; p-Hydroxymandelic acid; |
| | | 3,4-Dihydroxybenzeneacetic acid; 5- |
| | | Methoxysalicylic acid; Isovanillic acid |
| 168.0535 | C7H8N2O3 | 2,3-Diaminosalicylic acid |
| 169.0375 | C7H7NO4 | 2-Furoylglycine |
| 169.0739 | C8H11NO3 | Norepinephrine; Pyridoxine; 6- |
| | | Hydroxydopamine; 5-Hydroxydopamine |
| 169.0851 | C7H11N3O2 | 1-Methylhistidine; 3-Methylhistidine |
| 170.0167 | C4H11O3PS | Diethylthiophosphate |
| 170.0579 | C8H10O4 | 3,4-Dihydroxyphenylglycol; 3,4-Methyleneadipic |
| | | acid |
| 170.0732 | C12H10O | 2-Biphenylol |
| 170.1307 | C10H18O2 | Linalyl oxide |
| 172.0137 | C3H9O6P | Glycerol 3-phosphate |
| 172.0524 | C11H8O2 | Menadione |
| 172.0736 | C8H12O4 | 2-Octenedioic acid; cis-4-Octenedioic acid |
| 172.0848 | C7H12N2O3 | Glycylproline; L-prolyl-L-glycine |
| 172.1463 | C10H20O2 | Capric acid |
| 173.1052 | C8H15NO3 | Hexanoylglycine |
| 174.0164 | C6H6O6 | cis-Aconitic acid; trans-Aconitic acid; |
| | | Dehydroascorbic acid |
| 174.0528 | C7H10O5 | Shikimic acid |
| 174.0641 | C6H10N2O4 | Formiminoglutamic acid |
| 174.0892 | C8H14O4 | Suberic acid; 2,4-Dimethyladipic acid; 3- |
| | | Methylpimelic acid; 2-Propylglutaric acid |
| 174.1004 | C7H14N2O3 | N-Acetylornithine |
| 175.0481 | C6H9NO5 | N-Acetyl-L-aspartic acid |
| 175.0593 | C5H9N3O4 | Guanidinosuccinic acid |
| 175.0633 | C10H9NO2 | Indoleacetic acid; 5-Hydroxyindoleacetaldehyde |
| 175.0957 | C6H13N3O3 | Citrulline |
| 176.0950 | C10H12N2O | Serotonin; Cotinine |
| 177.0460 | C6H11NO3S | N-Formyl-L-methionine |
| 177.0790 | C10H11NO2 | 5-Hydroxytryptophol |
| 178.0412 | C5H10N2O3S | Cysteinylglycine |
| 178.0630 | C10H10O3 | 4-Methoxycinnamic acid |
| 178.1106 | C10H14N2O | Nicotine-1′-N-oxide; Glycinexylidide |
| 179.0443 | C6H5N5O2 | Isoxanthopterin |
| 179.0582 | C9H9NO3 | Hippuric acid |
| 179.0695 | C8H9N3O2 | Acetylisoniazid |
| 179.0794 | C6H13NO5 | Glucosamine |
| 180.0423 | C9H8O4 | 4-Hydroxyphenylpyruvic acid; Aspirin; Caffeic |
| | | acid |
| 180.0535 | C8H8N2O3 | Nicotinuric acid; Isonicotinylglycine; |
| | | Picolinoylglycine |
| 180.0634 | C6H12O6 | D-Glucose; D-Galactose; D-Mannose; |
| | | Myoinositol; D-Fructose; L-Sorbose; Scyllitol |
| 180.0786 | C10H12O3 | 3-Methoxybenzenepropanoic acid; |
| | | Propylparaben; 3-(3-Hydroxyphenyl)-2- |
| | | methylpropionic acid |
| 180.0899 | C9H12N2O2 | Tyrosinamide |
| 181.0600 | C6H7N5O2 | 8-Hydroxy-7-methylguanine |
| 181.0739 | C9H11NO3 | L-Tyrosine; o-Tyrosine |
| 182.0440 | C6H6N4O3 | 3-Methyluric acid; 9-Methyluric acid; 1- |
| | | Methyluric acid |
| 182.0579 | C9H10O4 | Homovanillic acid; 3,4-Dihydroxyhydrocinnamic |
| | | acid; Hydroxyphenyllactic acid; 3-(3- |
| | | Hydroxyphenyl)-3-hydroxypropanoic acid; 2,6- |
| | | Dimethoxybenzoic acid |
| 182.0790 | C6H14O6 | Galactitol; Sorbitol; Mannitol |
| 182.1307 | C11H18O2 | Methyl 4,8-decadienoate |
| 183.0532 | C8H9NO4 | 4-Pyridoxic acid |
| 183.0895 | C9H13NO3 | Epinephrine; Normetanephrine; Levonordefrin |
| 184.0372 | C8H8O5 | 3,4-Dihydroxymandelic acid; 4-O-Methylgallic |
| | | acid |
| 184.0736 | C9H12O4 | Vanylglycol; 3,4-Methylenepimelic acid |
| 185.0089 | C3H8NO6P | Phosphoserine; DL-O-Phosphoserine |
| 185.9929 | C3H7O7P | 2-Phospho-D-glyceric acid |
| 187.1685 | C9H21N3O | N1-Acetylspermidine; N8-Acetylspermidine |
| 188.0143 | C7H8O4S | p-Cresol sulfate |
| 188.0797 | C7H12N2O4 | N-Acetylglutamine |
| 188.1049 | C9H16O4 | Azelaic acid; 2,4-Dimethylpimelic acid; 3- |
| | | Methylsuberic acid |
| 188.1161 | C8H16N2O3 | N6-Acetyl-L-lysine; Glycyl-L-leucine |
| 188.1273 | C7H16N4O2 | Homo-L-arginine |
| 188.1313 | C12H16N2 | Dimethyltryptamine |
| 188.1525 | C9H20N2O2 | N6,N6,N6-Trimethyl-L-lysine |
| 189.0096 | C6H7NO4S | Lanthionine ketimine |
| 189.0426 | C10H7NO3 | Kynurenic acid |
| 189.0637 | C7H11NO5 | N-Acetylglutamic acid |
| 189.1113 | C7H15N3O3 | Homocitrulline |
| 190.0477 | C7H10O6 | 3-Dehydroquinate |
| 190.0841 | C8H14O5 | 3-Hydroxysuberic acid |
| 190.1106 | C11H14N2O | 5-Methoxytryptamine |
| 190.1358 | C13H18O | beta-Damascenone |
| 191.0582 | C10H9NO3 | 5-Hydroxyindoleacetic acid; 5-Phenyl-1,3- |
| | | oxazinane-2,4-dione |
| 192.0270 | C6H8O7 | Citric acid; Isocitric acid |
| 192.0899 | C10H12N2O2 | Hydroxycotinine; Cotinine N-oxide |
| 192.1514 | C13H20O | 4-(2,6,6-Trimethyl-1,3-cyclohexadien-1-yl)-2- |
| | | butanone |
| 193.0739 | C10H11NO3 | Phenylacetylglycine; 2-Methylhippuric acid; 3- |
| | | Carbamoyl-2-phenylpropionaldehyde; 4- |
| | | Hydroxy-5-phenyltetrahydro-1,3-oxazin-2-one; |
| | | 4-Anilino-4-oxobutanoic acid |
| 194.0427 | C6H10O7 | D-Glucuronic acid; Iduronic acid; Pectin |
| 194.0691 | C9H10N2O3 | 4-Aminohippuric acid |
| 194.0943 | C11H14O3 | Butylparaben |
| 194.1307 | C12H18O2 | 4-Hydroxypropofol |
| 195.0532 | C9H9NO4 | Salicyluric acid; 3-Hydroxyhippuric acid; 4- |
| | | Hydroxyhippuric acid; N-acetyl-5-aminosalicylic |
| | | acid |
| 196.0583 | C6H12O7 | Galactonic acid; Gluconic acid |
| 196.0596 | C7H8N4O3 | 1,3-Dimethyluric acid; 3,7-Dimethyluric acid; |
| | | 1,9-Dimethyluric acid; 7,9-Dimethyluric acid; |
| | | 1,7-Dimethyluric acid |
| 196.0736 | C10H12O4 | Homoveratric acid; 3-(3-Hydroxyphenyl)-2- |
| | | methyllactic acid; 3-(3,4-Dihydroxyphenyl)-2- |
| | | methylpropionic acid |
| 196.9955 | C5H11NO2Se | Selenomethionine |
| 197.0688 | C9H11NO4 | L-Dopa |
| 197.1052 | C10H15NO3 | Metanephrine; Desglymidodrine |
| 198.0325 | C5H12ClN2O2P | 3-Dechloroethylifosfamide; 2- |
| | | Dechloroethylifosfamide; Dechloroethyl |
| | | cyclophosphamide |
| 198.0528 | C9H10O5 | Vanillylmandelic acid; Syringic acid; 3,4-O- |
| | | Dimethylgallic acid |
| 198.0753 | C7H10N4O3 | 5-Acetylamino-6-amino-3-methyluracil; 6- |
| | | amino-5[N-methylformylamino]-1-methyluracil |
| 199.0246 | C4H10NO6P | O-Phosphothreonine |
| 200.1049 | C10H16O4 | cis-4-Decenedioic acid |
| 200.1776 | C12H24O2 | Dodecanoic acid |
| 202.1205 | C10H18O4 | Sebacic acid; 3-Methylazelaic acid |
| 202.1430 | C8H18N4O2 | Asymmetric dimethylarginine; Symmetric |
| | | dimethylarginine |
| 202.2157 | C10H26N4 | Spermine |
| 203.0252 | C7H9NO4S | Cystathionine ketimine |
| 203.1059 | C11H13N3O | Tryptophanamide; OR-1855 |
| 203.1158 | C9H17NO4 | L-Acetylcarnitine |
| 204.0899 | C11H12N2O2 | L-Tryptophan; 3-Hydroxymethylantipyrine; |
| | | Ethotoin; (_)-Tryptophan; Nirvanol; 4- |
| | | Hydroxyantipyrine; S-nirvanol |
| 205.0375 | C10H7NO4 | Xanthurenic acid |
| 205.0739 | C11H11NO3 | Indolelactic acid; 3-Indolehydracrylic acid |
| 206.0427 | C7H10O7 | 2-Methylcitric acid |
| 207.0895 | C11H13NO3 | Phenylpropionylglycine; N- |
| | | isopropylterephthalamic acid |
| 207.1008 | C10H13N3O2 | 4-(Methylnitrosamino)-1-(3-pyridyl)-1-butanone |
| 208.0736 | C11H12O4 | 5-(3′,4′-Dihydroxyphenyl)-gamma-valerolactone; |
| | | 3-(3,4-Dimethoxyphenyl)-2-propenoic acid; 5- |
| | | (3′,5′-Dihydroxyphenyl)-gamma-valerolactone |
| 208.0848 | C10H12N2O3 | L-Kynurenine; 4-Aminobenzoyl-(beta)-alanine |
| 209.0688 | C10H11NO4 | Hydroxyphenylacetylglycine; 3-Carbamoyl-2- |
| | | phenylpropionic acid |
| 209.1164 | C10H15N3O2 | 4-(Methylnitrosamino)-1-(3-pyridyl)-1-butanol |
| 210.0376 | C6H10O8 | Galactaric acid; Glucaric acid |
| 210.0528 | C10H10O5 | Vanilpyruvic acid |
| 210.0753 | C8H10N4O3 | 1,3,7-Trimethyluric acid |
| 211.0358 | C4H10N3O5P | Phosphocreatine |
| 211.0845 | C10H13NO4 | 3-Methoxytyrosine; Methyldopa; 3-O-Methyl-a- |
| | | methyldopa |
| 212.0473 | C13H8O3 | Urolithin B |
| 212.0685 | C10H12O5 | Vanillactic acid; 3-Hydroxy-4- |
| | | methoxyphenyllactic acid; beta-(2- |
| | | Methoxyphenoxy)-lactic acid |
| 213.0038 | C4H8NO7P | L-Aspartyl-4-phosphate |
| 213.0096 | C8H7NO4S | Indoxyl sulfate |
| 214.0776 | C9H14N2O2S | Methyl bisnorbiotinyl ketone |
| 215.0349 | C9H10ClNO3 | 3-Chlorotyrosine |
| 215.1158 | C10H17NO4 | Propenoylcarnitine |
| 216.0569 | C8H12N2O3S | Bisnorbiotin; 6-Aminopenicillanic acid |
| 216.0786 | C13H12O3 | O-Desmethylnaproxen |
| 216.1222 | C8H16N4O3 | N-a-Acetyl-L-arginine |
| 217.1215 | C12H15N3O | N-Desmethylaminopyrine |
| 217.1314 | C10H19NO4 | Propionylcarnitine |
| 218.1055 | C12H14N2O2 | N-Acetylserotonin; Mephenytoin; Primidone |
| 218.1154 | C10H18O5 | 3-Hydroxysebacic acid; 2-Hydroxydecanedioic |
| | | acid |
| 218.1419 | C13H18N2O | 5-Methoxydimethyltryptamine; N-despropyl |
| | | ropinirole |
| 219.1259 | C13H17NO2 | Ritalinic acid |
| 219.9694 | C8H6Cl2O3 | 2,4-Dichlorophenoxyacetic acid |
| 220.0670 | C11H12N2OS | Dehydroxyzyleuton |
| 220.0848 | C11H12N2O3 | 5-Hydroxy-L-tryptophan; Oxitriptan; p- |
| | | Hydroxyl-ethotoin |
| 220.0888 | C16H12O | 1-Hydroxypyrene |
| 220.1463 | C14H20O2 | 2,6-Di-tert-butylbenzoquinone |
| 221.0899 | C8H15NO6 | N-Acetyl-D-glucosamine |
| 222.0674 | C7H14N2O4S | L-Cystathionine |
| 222.0740 | C8H14O7 | Ethyl glucuronide |
| 222.0892 | C12H14O4 | Monoisobutyl phthalic acid; Monobutylphthalate; |
| | | 5′-(3′-Methoxy-4′-hydroxyphenyl)-gamma- |
| | | valerolactone |
| 223.0845 | C11H13NO4 | N-Acetyl-L-tyrosine |
| 224.0685 | C11H12O5 | Sinapic acid; 5-(3′,4′,5′-Trihydroxyphenyl)- |
| | | gamma-valerolactone |
| 224.0797 | C10H12N2O4 | Hydroxykynurenine; L-3-Hydroxykynurenine; |
| | | Stavudine |
| 226.0590 | C9H10N2O5 | 3-Nitrotyrosine |
| 226.0702 | C8H10N4O4 | 5-Acetylamino-6-formylamino-3-methyluracil |
| 226.0954 | C10H14N2O4 | Porphobilinogen |
| 226.1066 | C9H14N4O3 | Carnosine |
| 227.0906 | C9H13N3O4 | Deoxycytidine |
| 228.0423 | C13H8O4 | Urolithin A |
| 228.0746 | C9H12N2O5 | Deoxyuridine |
| 228.1110 | C10H16N2O4 | Prolylhydroxyproline |
| 228.1150 | C15H16O2 | Nabumetone; Bisphenol A |
| 228.1362 | C12H20O4 | Traumatic acid |
| 228.1474 | C11H20N2O3 | L-isoleucyl-L-proline; L-leucyl-L-proline; |
| | | Leucyl-Proline |
| 228.2089 | C14H28O2 | Myristic acid |
| 229.1314 | C11H19NO4 | Butenylcarnitine |
| 231.1107 | C10H17NO5 | Suberylglycine |
| 231.1471 | C11H21NO4 | Butyrylcarnitine |
| 232.1212 | C13H16N2O2 | Melatonin; Aminoglutethimide |
| 233.0358 | C8H11NO5S | Dopamine 4-sulfate; Dopamine 3-O-sulfate |
| 233.1263 | C10H19NO5 | Hydroxypropionylcarnitine |
| 233.9866 | C7H7ClN2O3S | p-Chlorobenzene sulfonyl urea |
| 234.1004 | C12H14N2O3 | S-4-Hydroxymephenytoin; 4′- |
| | | hydroxymephenytoin |
| 237.0862 | C9H11N5O3 | Biopterin |
| 238.0841 | C12H14O5 | 3,4,5-Trimethoxycinnamic acid |
| 240.0238 | C6H12N2O4S2 | L-Cystine |
| 240.0998 | C12H16O5 | 3-(3,4,5-Trimethoxyphenyl)propanoic acid |
| 240.1222 | C10H16N4O3 | Anserine; Homocarnosine |
| 242.0903 | C10H14N2O5 | Thymidine; Telbivudine |
| 242.0943 | C15H14O3 | Equol; Fenoprofen |
| 243.0855 | C9H13N3O5 | Cytidine; Cytarabine |
| 243.1471 | C12H21NO4 | Tiglylcarnitine |
| 244.0372 | C13H8O5 | Urolithin C |
| 244.0695 | C9H12N2O6 | Uridine; Pseudouridine |
| 244.2263 | C12H28N4O | N1-Acetylspermine |
| 245.1627 | C12H23NO4 | 2-Methylbutyroylcarnitine; Valerylcarnitine |
| 246.0852 | C9H14N2O6 | 5,6-Dihydrouridine |
| 246.1004 | C13H14N2O3 | N-acetyltryptophan; Methylphenobarbital; cyclic |
| | | 6-Hydroxymelatonin |
| 247.1056 | C10H17NO6 | Malonylcarnitine |
| 248.1161 | C13H16N2O3 | 6-Hydroxymelatonin; 2-Oxomelatonin |
| 249.0307 | C8H11NO6S | Norepinephrine sulfate |
| 250.0623 | C8H14N2O5S | Gamma-Glutamylcysteine |
| 251.1018 | C10H13N5O3 | Deoxyadenosine; 5′-Deoxyadenosine |
| 252.0569 | C11H12N2O3S | Hydroxyzileuton; Zileuton sulfoxide |
| 252.0859 | C10H12N4O4 | Deoxyinosine |
| 252.1110 | C12H16N2O4 | 3′-Hydroxyhexobarbital; Epoxy-hexobarbital |
| 253.0811 | C9H11N5O4 | Neopterin |
| 253.0870 | C13H16ClNO2 | 5-Hydroxyketamine; 4-Hydroxyketamine; 6- |
| | | Hydroxyketamine |
| 253.0950 | C12H15NO5 | N-Acetylvanilalanine |
| 254.2246 | C16H30O2 | Palmitoleic acid |
| 255.0981 | C11H15N2O5 | Nicotinamide riboside |
| 255.1026 | C13H18ClNO2 | Hydroxybupropion |
| 256.0736 | C15H12O4 | Dihydrodaidzein; 2-Dehydro-O- |
| | | desmethylangolensin |
| 256.2402 | C16H32O2 | Palmitic acid |
| 257.1012 | C10H15N3O5 | 5-Methylcytidine |
| 257.1627 | C13H23NO4 | 2-Hexenoylcarnitine |
| 257.1780 | C17H23NO | 3-Methoxymorphinan; Levorphanol; Dextrorphan |
| 258.0852 | C10H14N2O6 | Ribothymidine; 3-Methyluridine |
| 258.0892 | C15H14O4 | O-Desmethylangolensin; 3′,4′,7- |
| | | Trihydroxyisoflavan; 3′-Hydroxyequol; cis-4- |
| | | Hydroxyequol |
| 259.1784 | C13H25NO4 | L-Hexanoylcarnitine |
| 260.0297 | C6H13O9P | Glucose 6-phosphate |
| 261.0379 | C14H12ClNS | Dehydrogenated ticlopidine |
| 261.0402 | C9H12NO6P | O-Phosphotyrosine |
| 261.1212 | C11H19NO6 | Methylmalonylcarnitine |
| 262.0147 | C9H10O7S | Homovanillic acid sulfate; Dihydrocaffeic acid 3- |
| | | sulfate; 3-hydroxy-3-(3- |
| | | hydroxyphenyl)propanoic acid-O-sulphate |
| 262.0457 | C14H13ClNS | Thienodihydropyridinium |
| 263.0464 | C9H13NO6S | Epinephrine sulfate |
| 263.1885 | C16H25NO2 | N-Desmethylvenlafaxine; Tramadol; |
| | | Desvenlafaxine; O-Desmethylvenlafaxine |
| 264.0304 | C9H12O7S | 3-Methoxy-4-Hydroxyphenylglycol sulfate |
| 264.0780 | C9H16N2O5S | N-Acetylcystathionine |
| 264.1110 | C13H16N2O4 | Alpha-N-Phenylacetyl-L-glutamine; di- |
| | | Hydroxymelatonin |
| 265.1467 | C18H19NO | E-10-Hydroxydesmethylnortriptyline; N- |
| | | Desmethyldoxepin |
| 267.0954 | C9H17NO8 | Neuraminic acid |
| 267.0968 | C10H13N5O4 | Adenosine; Deoxyguanosine; Vidarabine; |
| | | Zidovudine |
| 268.0551 | C8H16N2O4S2 | DL-Homocystine; L-Homocystine |
| 268.0808 | C10H12N4O5 | Inosine |
| 269.0470 | C10H11N3O4S | Sulfamethoxazole N4-hydroxylamine; 5- |
| | | Hydroxysulfamethoxazole; sulfamethoxazole |
| | | hydroxylamine |
| 270.0119 | C7H14N2O4Se | Selenocystathionine |
| 270.0528 | C15H10O5 | Genistein; 6-Hydroxydaidzein; 8- |
| | | Hydroxydaidzein; 3′-Hydroxydaidzein |
| 270.1191 | C16H18N2S | N-Desmethylpromazine |
| 270.1620 | C18H22O2 | Estrone |
| 271.1208 | C16H17NO3 | Norhydromorphone; Normorphine |
| 272.0685 | C15H12O5 | Naringenin; Dihydrogenistein; 3′- |
| | | Hydroxydihydrodaidzein; 6- |
| | | Hydroxydihydrodaidzein; 8- |
| | | Hydroxydihydrodaidzein |
| 272.1049 | C16H16O4 | 5C-aglycone; 3′-O-Methylequol; 4′,7-Dihydroxy- |
| | | 3′-methoxyisoflavan; 4′,7-Dihydroxy-6- |
| | | methoxyisoflavan; 6-O-Methylequol |
| 272.1776 | C18H24O2 | Estradiol; 17a-Estradiol |
| 273.1001 | C15H15NO4 | L-Thyronine |
| 273.1212 | C12H19NO6 | Glutaconylcarnitine |
| 274.0590 | C13H10N2O5 | cis,trans-5′-Hydroxythalidomide; 5- |
| | | Hydroxythalidomide; Thalidomide arene oxide |
| 274.1780 | C14H26O5 | 3-Hydroxytetradecanedioic acid |
| 275.1270 | C14H17N3O3 | Alanyltryptophan |
| 275.1369 | C12H21NO6 | Glutarylcarnitine |
| 276.0197 | C7H15Cl2N2O3P | 4-Hydroxycyclophosphamide; 4- |
| | | Hydroxyifosfamide; Aldophosphamide; |
| | | Aldoifosfamide |
| 276.0780 | C10H16N2O5S | Biotin sulfone |
| 276.1321 | C11H20N2O6 | Saccharopine |
| 276.2089 | C18H28O2 | 19-Norandrosterone; 19-Noretiocholanolone |
| 277.0256 | C9H11NO7S | DOPA sulfate |
| 278.1002 | C11H18O8 | Isovalerylglucuronide |
| 278.1518 | C16H22O4 | Monoethylhexyl phthalic acid; Diisobutyl |
| | | phthalate |
| 278.1630 | C15H22N2O3 | Leucyl-phenylalanine |
| 279.0485 | C14H14ClNOS | 7-Hydroxyticlopidine; 2-Oxoticlopidine; |
| | | Ticlopidine S-oxide; Ticlopidine N-oxide |
| 279.1623 | C19H21NO | E-10-Hydroxynortriptyline; Doxepin |
| 280.2402 | C18H32O2 | Linoleic acid |
| 281.1124 | C11H15N5O4 | 1-Methyladenosine |
| 282.0964 | C11H14N4O5 | 1-Methylinosine |
| 282.1117 | C15H14N4O2 | 12-Hydroxynevirapine; 2-Hydroxynevirapine; 8- |
| | | Hydroxynevirapine; 3-Hydroxynevirapine |
| 282.2559 | C18H34O2 | Oleic acid |
| 283.0917 | C10H13N5O5 | Guanosine; 8-Hydroxy-deoxyguanosine |
| 283.1208 | C17H17NO3 | N-Phenylacetylphenylalanine |
| 283.1936 | C19H25NO | N-Dealkylated tolterodine; Levallorphan |
| 284.0757 | C10H12N4O6 | Xanthosine |
| 284.0896 | C13H16O7 | p-Cresol glucuronide |
| 284.2715 | C18H36O2 | Stearic acid |
| 285.0961 | C11H15N3O6 | N4-Acetylcytidine |
| 285.1940 | C15H27NO4 | 2-Octenoylcarnitine |
| 286.0954 | C15H14N2O4 | 3′,4′-Dihydrodiol; Phenytoin dihydrodiol |
| 286.0987 | C12H18N2O4S | 4-Hydroxy tolbutamide |
| 286.1569 | C18H22O3 | 2-Hydroxyestrone; 4-Hydroxyestrone |
| 286.1933 | C19H26O2 | Androstenedione |
| 286.2369 | C14H30N4O2 | N1,N12-Diacetylspermine |
| 287.1117 | C11H17N3O6 | N-Ribosylhistidine |
| 287.2097 | C15H29NO4 | L-Octanoylcarnitine |
| 288.0594 | C10H12N2O8 | Orotidine |
| 288.1725 | C18H24O3 | Estriol; 2-Hydroxyestradiol; 16b- |
| | | Hydroxyestradiol; 4-hydroxystradiol |
| 288.2089 | C19H28O2 | Dehydroepiandrosterone; Testosterone |
| 289.1314 | C16H19NO4 | Benzoyl ecgonine |
| 289.1525 | C13H23NO6 | 3-Methylglutarylcarnitine |
| 289.1678 | C17H23NO3 | Donepezil metabolite M4; Hyoscyamine; |
| | | Atropine |
| 290.1226 | C10H18N4O6 | Argininosuccinic acid |
| 290.1994 | C17H26N2O2 | Verapamil metabolite D-617; 3- |
| | | hydroxyropivacaine |
| 290.2246 | C19H30O2 | Androsterone; Etiocholanolone; |
| | | Dihydrotestosterone |
| 292.0285 | C10H13ClN2O4S | 2-Hydroxychlorpropamide; 3- |
| | | Hydroxychlorpropamide |
| 292.1019 | C9H16N4O7 | Canavaninosuccinate |
| 292.2402 | C19H32O2 | Androstanediol |
| 293.1780 | C20H23NO | E-10-Hydroxyamitriptyline |
| 294.1216 | C14H18N2O5 | Glutamylphenylalanine |
| 296.2351 | C18H32O3 | 13S-hydroxyoctadecadienoic acid |
| 297.0146 | C14H13C12NS | 2-Chloroticlopidine |
| 297.0896 | C11H15N5O3S | 5′-Methylthioadenosine |
| 297.1073 | C11H15N5O5 | 1-Methylguanosine; Nelarabine |
| 297.1212 | C14H19NO6 | Phenethylamine glucuronide |
| 298.0689 | C13H14O8 | Benzoyl glucuronide (Benzoic acid) |
| 298.1053 | C14H18O7 | 2-Phenylethanol glucuronide |
| 298.1205 | C18H18O4 | 7C-aglycone; Enterolactone |
| 299.2824 | C18H37NO2 | Sphingosine |
| 300.0706 | C10H12N4O7 | beta-D-3-Ribofuranosyluric acid |
| 300.1296 | C17H20N2OS | Promazine 5-sulfoxide |
| 300.1725 | C19H24O3 | 2-Methoxyestrone; Testolactone |
| 301.0468 | C8H15NO9S | N-Acetylgalactosamine 4-sulphate; N- |
| | | Acetylglucosamine 6-sulfate |
| 301.2253 | C16H31NO4 | 2,6 Dimethylheptanoyl carnitine; |
| | | Nonanoylcarnitine |
| 301.2981 | C18H39NO2 | Sphinganine |
| 302.1518 | C18H22O4 | Enterodiol; Masoprocol |
| 302.1882 | C19H26O3 | 2-Hydroxyestradiol-3-methyl ether; 2- |
| | | Methoxyestradiol |
| 302.2246 | C20H30O2 | Eicosapentaenoic acid; Methyltestosterone |
| 303.1219 | C15H17N3O4 | Indoleacetyl glutamine |
| 303.1682 | C14H25NO6 | Pimelylcarnitine |
| 304.0907 | C11H16N2O8 | N-Acetylaspartylglutamic acid |
| 304.2038 | C19H28O3 | 16a-Hydroxydehydroisoandrosterone; 11- |
| | | Ketoetiocholanolone; 6beta-Hydroxytestosterone |
| 304.2402 | C20H32O2 | Arachidonic acid; Drostanolone |
| 306.0295 | C15H11ClO5 | Pelargonidin |
| 306.2195 | C19H30O3 | 5-Androstenetriol; 11-Hydroxyandrosterone; |
| | | Oxandrolone |
| 307.0838 | C10H17N3O6S | Glutathione |
| 309.0849 | C14H15NO7 | Inodxyl glucuronide; Indoxyl glucuronide |
| 309.1060 | C11H19NO9 | N-Acetylneuraminic acid |
| 310.1481 | C19H19FN2O | N-Desmethylcitalopram; Desmethylcitalopram |
| 311.0116 | C14H11Cl2NO3 | 4′-Hydroxydiclofenac; 3′-Hydroxydiclofenac; 5- |
| | | Hydroxydiclofenac |
| 311.1230 | C12H17N5O5 | N2,N2-Dimethylguanosine |
| 312.1474 | C18H20N2O3 | Phenylalanylphenylalanine |
| 313.1162 | C14H19NO7 | Tyramine glucuronide |
| 313.2253 | C17H31NO4 | 9-Decenoylcarnitine |
| 314.0638 | C13H14O9 | 1-Salicylate glucuronide |
| 314.1882 | C20H26O3 | 4-oxo-Retinoic acid |
| 315.2410 | C17H33NO4 | Decanoylcarnitine |
| 316.2038 | C20H28O3 | 15-Deoxy-d-12,14-PGJ2; 4-Hydroxyretinoic |
| | | acid; all-trans-5,6-Epoxyretinoic acid; 18- |
| | | Hydroxyretinoic acid |
| 317.1627 | C18H23NO4 | Arbutamine; Cocaethylene; alpha-oxycodol; beta- |
| | | oxycodol |
| 320.1471 | C14H24O8 | Valproic acid glucuronide; Octanoylglucuronide |
| 320.2351 | C20H32O3 | 15(S)-HETE; 20-Hydroxyeicosatetraenoic acid; |
| | | 12-HETE |
| 320.2715 | C21H36O2 | Pregnanediol |
| 323.9570 | C8H11Cl3O7 | Trichloroethanol glucuronide |
| 324.0778 | C17H13ClN4O | Alpha-hydroxyalprazolam; 4-hydroxyalprazolam |
| 324.0998 | C19H16O5 | S-6-Hydroxywarfarin; S-4′-Hydroxywarfarin; R- |
| | | 4′-Hydroxywarfarin; R-6-Hydroxywarfarin; R- |
| | | 10-Hydroxywarfarin; R-8-Hydroxywarfarin; R-7- |
| | | Hydroxywarfarin |
| 324.1533 | C12H24N2O8 | Galactosylhydroxylysine |
| 327.0954 | C14H17NO8 | Acetaminophen glucuronide |
| 328.2402 | C22H32O2 | Docosahexaenoic acid |
| 328.2515 | C21H32N2O | Stanozolol |
| 329.0525 | C10H12N5O6P | Cyclic AMP |
| 329.1111 | C14H19NO8 | Dopamine glucuronide |
| 329.1627 | C19H23NO4 | (S)-Reticuline |
| 330.2195 | C21H30O3 | 17-Hydroxyprogesterone; 11-Hydroxy-delta-9- |
| | | THC; 7-beta-Hydroxy-delta-9-THC; 7-alpha- |
| | | Hydroxy-delta-9-THC; 8-Hydroxy-delta-9-THC; |
| | | 8-beta-Hydroxy-delta-9-THC; 9-alpha,10-alpha- |
| | | epoxyhexahydrocannabinol; 6(beta)- |
| | | hydroxyprogesterone; 6-beta- |
| | | hydroxyprogesterone |
| 330.2559 | C22H34O2 | Docosapentaenoic acid (22n-6) |
| 331.0991 | C16H17N3O3S | 5′-O-Desmethyl omeprazole |
| 331.1042 | C18H18FNO2S | R-95913 |
| 332.2351 | C21H32O3 | 16-a-Hydroxypregnenolone; 21- |
| | | Hydroxypregnenolone |
| 334.0566 | C11H15N2O8P | Nicotinamide ribotide |
| 334.2144 | C20H30O4 | Delta-12-Prostaglandin J2 |
| 334.2508 | C21H34O3 | Tetrahydrodeoxycorticosterone |
| 335.1329 | C12H21N3O8 | Aspartylglycosamine |
| 336.2301 | C20H32O4 | Leukotriene B4 |
| 337.0539 | C16H16ClNO3S | 2-Oxoclopidogrel |
| 337.0798 | C15H15NO8 | 2,8-Dihydroxyquinoline-beta-D-glucuronide; 3- |
| | | Indole carboxylic acid glucuronide |
| 338.1478 | C16H22N2O6 | Nicotine glucuronide |
| 339.0954 | C15H17NO8 | 6-Hydroxy-5-methoxyindole glucuronide; 5- |
| | | Hydroxy-6-methoxyindole glucuronide |
| 339.1253 | C15H21N3O4S | 7-Hydroxygliclazide; 6-Hydroxygliclazide; |
| | | Methylhydroxygliclazide |
| 339.1318 | C16H21NO7 | 5-Hydroxytryptophol glucuronide |
| 341.2566 | C19H35NO4 | trans-2-Dodecenoylcarnitine |
| 342.1162 | C12H22O11 | Melibiose; D-Maltose; Alpha-Lactose; Sucrose; |
| | | Trehalose |
| 342.2042 | C18H30O6 | 2,3-Dinor-6-keto-prostaglandin F1a; 2,3-Dinor- |
| | | TXB2; Monic acid |
| 343.2723 | C19H37NO4 | Dodecanoylcarnitine |
| 346.2144 | C21H30O4 | Cortexolone |
| 347.0576 | C15H13N3O5S | 5′-Hydroxypiroxicam |
| 347.0631 | C10H14N5O7P | Adenosine monophosphate |
| 349.1148 | C18H20FNO3S | R-138727 |
| 350.1188 | C18H22O5S | Estrone sulfate |
| 350.2457 | C21H34O4 | Tetrahydrocorticosterone; 5a- |
| | | Tetrahydrocorticosterone; |
| | | Tetrahydrodeoxycortisol |
| 352.1271 | C16H20N2O7 | Cotinine glucuronide |
| 352.1344 | C18H24O5S | Estradiol-17beta 3-sulfate |
| 352.2250 | C20H32O5 | Prostaglandin E2; Thromboxane A2; 20- |
| | | Hydroxy-leukotriene B4; 13,14-Dihydro-15-keto- |
| | | PGE2 |
| 353.0140 | C13H11N3O5S2 | 5′-Hydroxytenoxicam |
| 354.2406 | C20H34O5 | Prostaglandin F2a; 8-Isoprostaglandin F2a; 11b- |
| | | PGF2a |
| 357.1940 | C21H27NO4 | Nalbuphine; (S)-Laudanosine; 5- |
| | | hydroxypropafenone |
| 357.2093 | C25H27NO | N-Desmethyltamoxifen |
| 359.1216 | C15H21NO9 | Epinephrine glucuronide |
| 360.0845 | C18H16O8 | Rosmarinic acid |
| 360.1056 | C15H20O10 | 3-Methoxy-4-hydroxyphenylglycol glucuronide |
| 360.1321 | C18H20N2O6 | Dityrosine; Nitrendipine |
| 360.1937 | C21H28O5 | Aldosterone; Cortisone; Prednisolone |
| 361.1096 | C17H19N3O4S | 5-Hydroxyomeprazole; Omeprazole sulfone; 3- |
| | | Hydroxyomeprazole; 5-hydroxyesomeprazole |
| 362.2093 | C21H30O5 | Cortisol; Hydrocortisone |
| 364.2250 | C21H32O5 | Tetrahydrocortisone |
| 365.1059 | C18H21O6S | 2-Hydroxyestrone sulfate |
| 365.1991 | C23H27NO3 | 5-O-Desmethyldonepezil; 6-O- |
| | | Desmethyldonepezil |
| 366.1137 | C18H22O6S | 4-Hydroxyestrone sulfate |
| 366.2042 | C20H30O6 | 20-Carboxy-leukotriene B4 |
| 366.2406 | C21H34O5 | 5a-Tetrahydrocortisol; Tetrahydrocortisol; |
| | | Cortolone |
| 367.2723 | C21H37NO4 | 3,5-Tetradecadiencarnitine |
| 368.1220 | C16H20N2O8 | trans-3-Hydroxycotinine glucuronide |
| 368.1558 | C21H24N2O2S | N-Desmethyleletriptan |
| 368.1657 | C19H28O5S | Dehydroepiandrosterone sulfate; Testosterone |
| | | sulfate |
| 368.2199 | C20H32O6 | 6,15-Diketo,13,14-dihydro-PGF1a; 11-Dehydro- |
| | | thromboxane B2 |
| 368.2563 | C21H36O5 | Cortol; Beta-Cortol |
| 369.0280 | C15H13O9S | (−)-Epicatechin sulfate |
| 369.1376 | C21H20FNO4 | N-Deisopropyl-fluvastatin |
| 369.2879 | C21H39NO4 | cis-5-Tetradecenoylcarnitine; trans-2- |
| | | Tetradecenoylcarnitine |
| 370.1814 | C19H30O5S | Androsterone sulfate; 5a-Dihydrotestosterone |
| | | sulfate |
| 370.2355 | C20H34O6 | 6-Keto-prostaglandin F1a; Thromboxane B2 |
| 371.3036 | C21H41NO4 | Tetradecanoylcarnitine |
| 374.0492 | C18H15ClN2O3S | 6-Hydroxymethyletoricoxib; Etoricoxib 1′-N′- |
| | | oxide |
| 374.1114 | C18H18N2O7 | Portulacaxanthin II |
| 374.2457 | C23H34O4 | Calcitroic acid |
| 374.2821 | C24H38O3 | 3b-Hydroxy-5-cholenoic acid |
| 376.2977 | C24H40O3 | Lithocholic acid |
| 378.1315 | C19H22O8 | 3,4-DHPEA-EA |
| 378.2042 | C21H30O6 | 18-Hydroxycortisol; 6-beta-hydrocortisol |
| 382.0359 | C16H14O9S | hesperetin 3′-O-sulfate |
| 383.1077 | C14H17N5O8 | Succinyladenosine |
| 383.2672 | C21H37NO5 | 3-Hydroxy-5, 8-tetradecadiencarnitine |
| 384.1216 | C14H20N6O5S | S-Adenosylhomocysteine |
| 385.0708 | C16H14F3N3O3S | Hydroxylansoprazole; Lansoprazole sulfone; 5- |
| | | hydroxylansoprazole |
| 385.2828 | C21H39NO5 | 3-Hydroxy-cis-5-tetradecenoylcarnitine |
| 386.9750 | C13H10ClN3O5S2 | 5′-Hydroxylornoxicam |
| 387.2198 | C26H29NO2 | 3-Hydroxytamoxifen (Droloxifene); Tamoxifen |
| | | N-oxide; 4-Hydroxytamoxifen; alpha- |
| | | Hydroxytamoxifen |
| 389.0723 | C11H20NO12P | N-Acetylneuraminate 9-phosphate |
| 390.1063 | C18H18N2O8 | Dopaxanthin |
| 392.1736 | C23H24N2O4 | O-Desmethylcarvedilol |
| 392.2927 | C24H40O4 | Chenodeoxycholic acid; Deoxycholic acid; |
| | | Isoursodeoxycholic acid; Hyodeoxycholic acid; |
| | | Ursodeoxycholic acid |
| 394.2484 | C19H39O6P | LPA(P-16:0e/0:0) |
| 395.3036 | C23H41NO4 | 9,12-Hexadecadienoylcarnitine |
| 396.1970 | C21H32O5S | Pregnenolone sulfate; 3beta-Hydroxypregn-5-en- |
| | | 20-one sulfate |
| 397.0708 | C17H14F3N3O3S | Hydroxycelecoxib |
| 399.1451 | C15H23N6O5S | S-Adenosylmethionine |
| 399.3349 | C23H45NO4 | L-Palmitoylcarnitine |
| 400.3341 | C27H44O2 | 7a-Hydroxy-cholestene-3-one; Calcidiol; |
| | | Alfacalcidol |
| 408.2876 | C24H40O5 | 1b,3a,12a-Trihydroxy-5b-cholanoic acid; Cholic |
| | | acid; Hyocholic acid; Ursocholic acid |
| 410.1905 | C22H28F2O5 | Tafluprost free acid; Diflorasone |
| 412.1284 | C22H16N6O3 | O-Deethylated candesartan |
| 412.1920 | C21H32O6S | 17-Hydroxypregnenolone sulfate |
| 412.1958 | C18H28N4O7 | Deoxypyridinoline |
| 412.3341 | C28H44O2 | 25-Hydroxyvitamin D2 |
| 413.0464 | C14H15N5O6S2 | Desacetylcefotaxime |
| 415.3298 | C23H45NO5 | 3-Hydroxyhexadecanoylcarnitine |
| 418.2931 | C22H42O7 | Palmitoyl glucuronide |
| 420.1465 | C22H21ClN6O | E-3179 |
| 422.1842 | C24H26N2O5 | 8-Hydroxycarvedilol; 4′-Hydroxycarvedilol; 5′- |
| | | Hydroxycarvedilol; 1-Hydroxycarvedilol; 4′- |
| | | Hydroxyphenyl Carvedilol |
| 423.3349 | C25H45NO4 | Linoleyl carnitine |
| 427.0294 | C10H15N5O10P2 | ADP |
| 427.1795 | C24H26FNO5 | 6-Hydroxyfluvastatin; 5-Hydroxyfluvastatin |
| 428.1907 | C18H28N4O8 | Pyridinoline |
| 429.0842 | C16H19N3O9S | Sulfamethoxazole N1-glucuronide |
| 430.1264 | C22H22O9 | Ketoprofen glucuronide |
| 432.0913 | C18H24O8S2 | 17-Beta-Estradiol-3,17-beta-sulfate |
| 432.8672 | C9H9I2NO3 | 3,5-Diiodo-L-tyrosine |
| 433.1373 | C21H23NO9 | Tolmetin glucuronide |
| 433.2101 | C23H31NO7 | Dextrorphan O-glucuronide; Mycophenolate |
| | | mofetil |
| 439.2392 | C23H37NO5S | Leukotriene E4 |
| 440.2675 | C26H36N2O4 | O-Desmethylverapamil (D-702); O- |
| | | Desmethylverapamil (D-703); Norverapamil |
| 445.1710 | C19H23N7O6 | Tetrahydrofolic acid |
| 446.1941 | C24H30O8 | Estrone glucuronide |
| 446.2430 | C25H30N6O2 | SR 49498 |
| 448.2097 | C24H32O8 | 2-Methoxyestrone 3-glucuronide; 17-beta- |
| | | Estradiol-3-glucuronide; 17-beta-Estradiol |
| | | glucuronide; 17-alpha-Estradiol-3-glucuronide; |
| | | Estradiol-17alpha 3-D-glucuronoside |
| 449.1084 | C21H21O11 | Cyanidin 3-glucoside; Cyanidin 3-galactoside |
| 449.3141 | C26H43NO5 | Deoxycholic acid glycine conjugate; |
| | | Chenodeoxycholic acid glycine conjugate; |
| | | Glycoursodeoxycholic acid |
| 451.2220 | C24H29N5O4 | 4-Hydroxyvalsartan |
| 454.0737 | C13H19N4O12P | SAICAR |
| 459.1866 | C20H25N7O6 | 5-Methyltetrahydrofolic acid |
| 460.1482 | C22H24N2O9 | Oxytetracycline |
| 461.1686 | C23H27NO9 | Morphine-3-glucuronide; Morphine-6- |
| | | glucuronide; Hydromorphone-3-glucuronide; |
| | | Hydromorphone 3-beta-O-glucuronide |
| 462.0798 | C21H18O12 | Kaempferol 3-glucuronide |
| 462.0830 | C21H19ClN2O8 | Oxazepam glucuronide |
| 462.2254 | C25H34O8 | 6-Dehydrotestosterone glucuronide |
| 462.2618 | C26H38O7 | Retinyl beta-glucuronide |
| 463.1240 | C22H23O11 | Peonidin-3-glucoside |
| 464.2046 | C24H32O9 | Estriol-16-Glucuronide; Estriol-17-glucuronide; |
| | | Estriol-3-glucuronide; 15-Hydroxynorandrostene- |
| | | 3,17-dione glucuronide; 16-alpha,17-beta-estriol |
| | | 17-beta-D-glucuronide |
| 464.2410 | C25H36O8 | Testosterone glucuronide; |
| | | Dehydroisoandrosterone 3-glucuronide; |
| | | Dehydroepiandrosterone 3-glucuronide |
| 465.3090 | C26H43NO6 | Glycocholic acid |
| 466.2567 | C25H38O8 | Androsterone glucuronide; Etiocholanolone |
| | | glucuronide; 5-alpha-Dihydrotestosterone |
| | | glucuronide; 3-alpha-hydroxy-5-alpha- |
| | | androstane-17-one 3-D-glucuronide |
| 468.2723 | C25H40O8 | 3,17-Androstanediol glucuronide; 3-alpha- |
| | | Androstanediol glucuronide; 17- |
| | | Hydroxyandrostane-3-glucuronide |
| 476.2410 | C26H36O8 | Retinoyl b-glucuronide |
| 478.0747 | C21H18O13 | Quercetin 3-O-glucuronide; Quercetin-4′- |
| | | glucuronide; Quercetin 4′-glucuronide; Quercetin |
| | | 3′-O-glucuronide |
| 478.2203 | C25H34O9 | 2-Methoxy-estradiol-17b 3-glucuronide; 4- |
| | | Hydroxyandrostenedione glucuronide |
| 479.2434 | C28H29N7O | N-desmethylimatinib; n-Demethylated piperazine |
| 480.2359 | C25H36O9 | 11-Oxo-androsterone glucuronide |
| 481.2498 | C25H39NO6S | N-Acetyl-leukotriene E4 |
| 482.2516 | C25H38O9 | 11-beta-Hydroxyandrosterone-3-glucuronide |
| 486.2061 | C18H34N2O13 | Glucosylgalactosyl hydroxylysine |
| 493.1346 | C23H25O12 | Malvidin 3-glucoside; Malvidin 3-galactoside |
| 493.3168 | C24H48NO7P | LysoPC(16:1(9Z)) |
| 495.3325 | C24H50NO7P | LysoPC(16:0) |
| 496.0440 | C21H18Cl2N2O8 | Lorazepam glucuronide |
| 496.3036 | C27H44O8 | Pregnanediol-3-glucuronide; 3-alpha,20-alpha- |
| | | Dihydroxy-5-beta-pregnane 3-glucuronide |
| 499.2968 | C26H45NO6S | Taurochenodesoxycholic acid |
| 504.1690 | C18H32O16 | Maltotriose |
| 506.2516 | C27H38O9 | 11-Hydroxyprogesterone 11-glucuronide |
| 509.2539 | C29H31N7O2 | CGP71422 |
| 509.3481 | C25H52NO7P | LysoPC(17:0) |
| 510.2617 | C29H32N7O2 | CGP72383 |
| 511.2696 | C29H33N7O2 | AFN911 |
| 513.2760 | C26H43NO7S | Sulfolithocholylglycine |
| 515.2917 | C26H45NO7S | Taurocholic acid; Taurohyocholate |
| 519.3325 | C26H50NO7P | LysoPC(18:2(9Z,12Z)) |
| 523.3638 | C26H54NO7P | LysoPC(18:0) |
| 524.8934 | C15H13I2NO4 | 3,5-Diiodothyronine |
| 526.2877 | C24H40N5O8 | Desmosine; Isodesmosine |
| 528.1665 | C24H32O11S | 17-beta-estradiol 3-sulfate-17-(beta-D- |
| | | glucuronide) |
| 536.1166 | C24H24O14 | Jaceidin 4′-glucuronide; 3,5,6-Trihydroxy-3′,4′,7- |
| | | trimethoxyflavone 3-glucuronide |
| 536.2258 | C27H36O11 | Aldosterone 18-glucuronide |
| 540.2571 | C27H40O11 | Tetrahydroaldosterone-3-glucuronide |
| 542.2727 | C27H42O11 | Cortolone-3-glucuronide |
| 543.3325 | C28H50NO7P | LysoPC(20:4(5Z,8Z,11Z,14Z)); |
| | | LysoPC(20:4(8Z,11Z,14Z,17Z)) |
| 544.1614 | C24H32O12S | Estriol 3-sulfate 16-glucuronide |
| 545.1897 | C27H31NO11 | Doxorubicinol; Doxorubicin-semiquinone; |
| | | Doxorubicinol aglycone |
| 552.3298 | C30H48O9 | Lithocholate 3-O-glucuronide |
| 562.3870 | C33H54O7 | Cholesterol glucuronide |
| 566.0550 | C15H24N2O17P2 | Uridine diphosphate glucose; Uridine |
| | | diphosphategalactose |
| 568.3247 | C30H48O10 | Deoxycholic acid 3-glucuronide |
| 572.3713 | C34H52O7 | Vitamin D2 3-glucuronide |
| 584.2635 | C33H36N4O6 | Bilirubin |
| 584.3197 | C30H48O11 | Cholic acid glucuronide |
| 588.2948 | C33H40N4O6 | D-Urobilin |
| 588.3662 | C34H52O8 | 25-Hydroxyvitamin D2-25-glucuronide; 25- |
| | | Hydroxyvitamin D2 25-(beta-glucuronide) |
| 590.3104 | C33H42N4O6 | D-Urobilinogen |
| 594.3417 | C33H46N4O6 | L-Urobilin |
| 595.1663 | C27H31O15 | Cyanidin 3-rutinoside; Peonidin 3-sambubioside |
| 598.1943 | C28H31ClN6O7 | Losartan N2-glucuronide |
| 606.3404 | C33H50O10 | (23S)-23,25-dihdroxy-24-oxovitamine D3 23- |
| | | (beta-glucuronide) |
| 612.3873 | C33H56O10 | Cholestane-3,7,12,25-tetrol-3-glucuronide |
| 616.1918 | C30H28N6O9 | Candesartan N2-glucuronide; Candesartan O- |
| | | glucuronide |
| 625.3462 | C32H51NO11 | Glycochenodeoxycholic acid 3-glucuronide |
| 633.2116 | C23H39NO19 | 3′-Sialyllactose |
| 634.4081 | C36H58O9 | Soyasapogenol B 3-O-b-D-glucuronide |
| 641.3411 | C32H51NO12 | (3a,5b,7a,12a)-24-[(carboxymethyl)amino]-1,12- |
| | | dihydroxy-24-oxocholan-3-yl-b-D- |
| | | Glucopyranosiduronic acid |
| 646.3717 | C36H54O10 | Gypsogenin 3-O-b-D-glucuronide |
| 650.7900 | C15H12I3NO4 | Liothyronine |
| 654.2690 | C36H38N4O8 | Coproporphyrin III; Coproporphyrin I |
| 659.8614 | C6H18O24P6 | Myo-inositol hexakisphosphate |
| 665.3047 | C33H47NO13 | Natamycin |
| 666.2219 | C24H42O21 | Glycogen; Maltotetraose |
| 674.2382 | C25H42N2O19 | 3-Sialyl-N-acetyllactosamine |
| 675.4839 | C36H70NO8P | PC(14:0/14:1(9Z)); PC(14:1(9Z)/14:0) |
| 678.3615 | C36H54O12 | Medicagenic acid 3-O-b-D-glucuronide |
| 687.5203 | C38H74NO7P | PC(o-16:1(9Z)/14:1(9Z)) |
| 688.5155 | C37H73N2O7P | SM(d18:0/14:1(9Z)(OH)) |
| 689.5359 | C38H76NO7P | PC(o-14:0/16:1(9Z)) |
| 691.5516 | C38H78NO7P | PC(o-14:0/16:0) |
| 701.4996 | C38H72NO8P | PC(14:1(9Z)/16:1(9Z)); PC(16:1(9Z)/14:1(9Z)) |
| 702.5676 | C39H79N2O6P | SM(d18:0/16:1(9Z)) |
| 703.5754 | C39H80N2O6P | SM(d18:1/16:0) |
| 715.5516 | C40H78NO7P | PC(14:0/P-18:1(11Z)); PC(14:0/P-18:1(9Z)); |
| | | PC(14:1(9Z)/P-18:0); PC(16:1(9Z)/P-16:0); |
| | | PC(P-16:0/16:1(9Z)); PC(P-18:0/14:1(9Z)); |
| | | PC(P-18:1(11Z)/14:0); PC(P-18:1(9Z)/14:0); |
| | | PC(o-16:1(9Z)/16:1(9Z)) |
| 716.5468 | C39H77N2O7P | SM(d18:0/16:1(9Z)(OH)) |
| 717.5672 | C40H80NO7P | PC(14:0/P-18:0); PC(16:0/P-16:0); PC(P- |
| | | 16:0/16:0); PC(P-18:0/14:0); PC(o- |
| | | 16:0/16:1(9Z)) |
| 727.5152 | C40H74NO8P | PC(14:0/18:3(6Z,9Z,12Z)); |
| | | PC(14:0/18:3(9Z,12Z,15Z)); |
| | | PC(14:1(9Z)/18:2(9Z,12Z)); |
| | | PC(18:2(9Z,12Z)/14:1(9Z)); |
| | | PC(18:3(6Z,9Z,12Z)/14:0); |
| | | PC(18:3(9Z,12Z,15Z)/14:0) |
| 729.5309 | C40H76NO8P | PC(14:0/18:2(9Z,12Z)) |
| 730.7469 | C15H12I3NO7S | Triiodothyronine sulfate |
| 731.5465 | C40H78NO8P | PC(14:0/18:1(11Z)); PC(14:0/18:1(9Z)); |
| | | PC(14:1(9Z)/18:0); PC(16:0/16:1(9Z)); |
| | | PC(16:1(9Z)/16:0); PC(18:0/14:1(9Z)); |
| | | PC(18:1(11Z)/14:0); PC(18:1(9Z)/14:0) |
| 731.6067 | C41H84N2O6P | SM(d18:1/18:0) |
| 733.5622 | C40H80NO8P | PC(16:0/16:0); PC(14:0/18:0); PC(18:0/14:0) |
| 737.5359 | C42H76NO7P | PC(18:4(6Z,9Z,12Z,15Z)/P-16:0); PC(P- |
| | | 16:0/18:4(6Z,9Z,12Z,15Z)) |
| 739.5516 | C42H78NO7P | PC(18:3(6Z,9Z,12Z)/P-16:0); |
| | | PC(18:3(9Z,12Z,15Z)/P-16:0); PC(P- |
| | | 16:0/18:3(6Z,9Z,12Z)); PC(P- |
| | | 16:0/18:3(9Z,12Z,15Z)) |
| 741.5672 | C42H80NO7P | PC(16:1(9Z)/P-18:1(11Z)); PC(16:1(9Z)/P- |
| | | 18:1(9Z)); PC(18:2(9Z,12Z)/P-16:0); PC(P- |
| | | 16:0/18:2(9Z,12Z)); PC(P-18:1(11Z)/16:1(9Z)); |
| | | PC(P-18:1(9Z)/16:1(9Z)); PC(o- |
| | | 16:1(9Z)/18:2(9Z,12Z)) |
| 745.5985 | C42H84NO7P | PC(o-16:1(9Z)/18:0); PC(o-18:1(11Z)/16:0); |
| | | PC(o-18:1(9Z)/16:0) |
| 747.6142 | C42H86NO7P | PC(o-16:0/18:0) |
| 753.5309 | C42H76NO8P | PC(14:0/20:4(5Z,8Z,11Z,14Z)); |
| | | PC(14:0/20:4(8Z,11Z,14Z,17Z)); |
| | | PC(14:1(9Z)/20:3(5Z,8Z,11Z)); |
| | | PC(14:1(9Z)/20:3(8Z,11Z,14Z)); |
| | | PC(16:0/18:4(6Z,9Z,12Z,15Z)); |
| | | PC(16:1(9Z)/18:3(6Z,9Z,12Z)); |
| | | PC(16:1(9Z)/18:3(9Z,12Z,15Z)); |
| | | PC(18:3(6Z,9Z,12Z)/16:1(9Z)); |
| | | PC(18:3(9Z,12Z,15Z)/16:1(9Z)); |
| | | PC(18:4(6Z,9Z,12Z,15Z)/16:0); |
| | | PC(20:3(5Z,8Z,11Z)/14:1(9Z)); |
| | | PC(20:3(8Z,11Z,14Z)/14:1(9Z)); |
| | | PC(20:4(5Z,8Z,11Z,14Z)/14:0); |
| | | PC(20:4(8Z,11Z,14Z,17Z)/14:0) |
| 755.5465 | C42H78NO8P | PC(14:0/20:3(5Z,8Z,11Z)); |
| | | PC(14:0/20:3(8Z,11Z,14Z)); |
| | | PC(14:1(9Z)/20:2(11Z,14Z)); |
| | | PC(16:0/18:3(6Z,9Z,12Z)); |
| | | PC(16:0/18:3(9Z,12Z,15Z)); |
| | | PC(16:1(9Z)/18:2(9Z,12Z)); |
| | | PC(18:2(9Z,12Z)/16:1(9Z)); |
| | | PC(18:3(6Z,9Z,12Z)/16:0); |
| | | PC(18:3(9Z,12Z,15Z)/16:0); |
| | | PC(20:2(11Z,14Z)/14:1(9Z)); |
| | | PC(20:3(5Z,8Z,11Z)/14:0); |
| | | PC(20:3(8Z,11Z,14Z)/14:0) |
| 757.5622 | C42H80NO8P | PC(14:0/20:2(11Z,14Z)); |
| | | PC(14:1(9Z)/20:1(11Z)); PC(16:0/18:2(9Z,12Z)); |
| | | PC(16:1(9Z)/18:1(11Z)); PC(16:1(9Z)/18:1(9Z)); |
| | | PC(18:1(11Z)/16:1(9Z)); PC(18:1(9Z)/16:1(9Z)); |
| | | PC(18:2(9Z,12Z)/16:0); PC(20:1(11Z)/14:1(9Z)); |
| | | PC(20:2(11Z,14Z)/14:0) |
| 759.5778 | C42H82NO8P | PC(14:0/20:1(11Z)); PC(14:1(9Z)/20:0); |
| | | PC(16:0/18:1(11Z)); PC(16:0/18:1(9Z)); |
| | | PC(16:1(9Z)/18:0); PC(18:0/16:1(9Z)); |
| | | PC(18:1(11Z)/16:0); PC(18:1(9Z)/16:0); |
| | | PC(20:0/14:1(9Z)); PC(20:1(11Z)/14:0) |
| 760.2956 | C39H44N4O12 | Bilirubin glucuronide |
| 765.5672 | C44H80NO7P | PC(18:3(6Z,9Z,12Z)/P-18:1(11Z)); |
| | | PC(18:3(6Z,9Z,12Z)/P-18:1(9Z)); |
| | | PC(18:3(9Z,12Z,15Z)/P-18:1(11Z)); |
| | | PC(18:3(9Z,12Z,15Z)/P-18:1(9Z)); |
| | | PC(18:4(6Z,9Z,12Z,15Z)/dm18:0); |
| | | PC(20:4(5Z,8Z,11Z,14Z)/P-16:0); |
| | | PC(20:4(8Z,11Z,14Z,17Z)/P-16:0); PC(P- |
| | | 16:0/20:4(8Z,11Z,14Z,17Z)); PC(P- |
| | | 18:0/18:4(6Z,9Z,12Z,15Z)); PC(P- |
| | | 18:1(11Z)/18:3(6Z,9Z,12Z)); PC(P- |
| | | 18:1(11Z)/18:3(9Z,12Z,15Z)); PC(P- |
| | | 18:1(9Z)/18:3(6Z,9Z,12Z)); PC(P- |
| | | 18:1(9Z)/18:3(9Z,12Z,15Z)); PC(o- |
| | | 16:1(9Z)/20:4(8Z,11Z,14Z,17Z)) |
| 767.5829 | C44H82NO7P | PC(18:2(9Z,12Z)/P-18:1(11Z)); |
| | | PC(18:2(9Z,12Z)/P-18:1(9Z)); |
| | | PC(18:3(6Z,9Z,12Z)/P-18:0); |
| | | PC(18:3(9Z,12Z,15Z)/P-18:0); |
| | | PC(20:3(5Z,8Z,11Z)/P-16:0); |
| | | PC(20:3(8Z,11Z,14Z)/P-16:0); PC(P- |
| | | 16:0/20:3(5Z,8Z,11Z)); PC(P- |
| | | 16:0/20:3(8Z,11Z,14Z)); PC(P- |
| | | 18:0/18:3(6Z,9Z,12Z)); PC(P- |
| | | 18:0/18:3(9Z,12Z,15Z)); PC(P- |
| | | 18:1(11Z)/18:2(9Z,12Z)); PC(P- |
| | | 18:1(9Z)/18:2(9Z,12Z)); PC(o- |
| | | 16:0/20:4(8Z,11Z,14Z,17Z)); PC(o- |
| | | 18:2(9Z,12Z)/18:2(9Z,12Z)) |
| 769.5985 | C44H84NO7P | PC(18:1(11Z)/P-18:1(11Z)); PC(18:1(11Z)/P- |
| | | 18:1(9Z)); PC(18:1(9Z)/P-18:1(11Z)); |
| | | PC(18:1(9Z)/P-18:1(9Z)); PC(18:2(9Z,12Z)/P- |
| | | 18:0); PC(20:2(11Z,14Z)/P-16:0); PC(P- |
| | | 16:0/20:2(11Z,14Z)); PC(P-18:0/18:2(9Z,12Z)); |
| | | PC(P-18:1(11Z)/18:1(11Z)); PC(P- |
| | | 18:1(11Z)/18:1(9Z)); PC(P-18:1(9Z)/18:1(11Z)); |
| | | PC(P-18:1(9Z)/18:1(9Z)); PC(o- |
| | | 18:1(11Z)/18:2(9Z,12Z)); PC(o- |
| | | 18:1(9Z)/18:2(9Z,12Z)) |
| 771.6142 | C44H86NO7P | PC(18:0/P-18:1(11Z)); PC(18:0/P-18:1(9Z)); |
| | | PC(18:1(11Z)/P-18:0); PC(18:1(9Z)/P-18:0); |
| | | PC(20:1(11Z)/P-16:0); PC(P-16:0/20:1(11Z)); |
| | | PC(P-18:0/18:1(11Z)); PC(P-18:0/18:1(9Z)); |
| | | PC(P-18:1(11Z)/18:0); PC(P-18:1(9Z)/18:0); |
| | | PC(o-18:0/18:2(9Z,12Z)); PC(o- |
| | | 18:1(9Z)/18:1(11Z)) |
| 773.6298 | C44H88NO7P | PC(o-16:1(9Z)/20:0); PC(o-18:1(9Z)/18:0) |
| 775.6455 | C44H90NO7P | PC(o-16:0/20:0) |
| 776.6867 | C15H11I4NO4 | Thyroxine; Dextrothyroxine |
| 777.5309 | C44H76NO8P | PC(14:0/22:6(4Z,7Z,10Z,13Z,16Z,19Z)); |
| | | PC(14:1(9Z)/22:5(4Z,7Z,10Z,13Z,16Z)); |
| | | PC(14:1(9Z)/22:5(7Z,10Z,13Z,16Z,19Z)); |
| | | PC(16:1(9Z)/20:5(5Z,8Z,11Z,14Z,17Z)); |
| | | PC(18:2(9Z,12Z)/18:4(6Z,9Z,12Z,15Z)); |
| | | PC(18:3(6Z,9Z,12Z)/18:3(6Z,9Z,12Z)); |
| | | PC(18:3(6Z,9Z,12Z)/18:3(9Z,12Z,15Z)); |
| | | PC(18:3(9Z,12Z,15Z)/18:3(6Z,9Z,12Z)); |
| | | PC(18:3(9Z,12Z,15Z)/18:3(9Z,12Z,15Z)); |
| | | PC(18:4(6Z,9Z,12Z,15Z)/18:2(9Z,12Z)); |
| | | PC(20:5(5Z,8Z,11Z,14Z,17Z)/16:1(9Z)); |
| | | PC(22:5(4Z,7Z,10Z,13Z,16Z)/14:1(9Z)); |
| | | PC(22:5(7Z,10Z,13Z,16Z,19Z)/14:1(9Z)); |
| | | PC(22:6(4Z,7Z,10Z,13Z,16Z,19Z)/14:0) |
| 779.5465 | C44H78NO8P | PC(14:0/22:5(4Z,7Z,10Z,13Z,16Z)) |
| 780.6145 | C45H85N2O6P | SM(d18:0/22:3(10Z,13Z,16Z)) |
| 781.5622 | C44H80NO8P | PC(14:0/22:4(7Z,10Z,13Z,16Z)); |
| | | PC(16:0/20:4(5Z,8Z,11Z,14Z)); |
| | | PC(16:0/20:4(8Z,11Z,14Z,17Z)); |
| | | PC(16:1(9Z)/20:3(5Z,8Z,11Z)); |
| | | PC(16:1(9Z)/20:3(8Z,11Z,14Z)); |
| | | PC(18:0/18:4(6Z,9Z,12Z,15Z)); |
| | | PC(18:1(11Z)/18:3(6Z,9Z,12Z)); |
| | | PC(18:1(11Z)/18:3(9Z,12Z,15Z)); |
| | | PC(18:1(9Z)/18:3(6Z,9Z,12Z)); |
| | | PC(18:1(9Z)/18:3(9Z,12Z,15Z)); |
| | | PC(18:2(9Z,12Z)/18:2(9Z,12Z)); |
| | | PC(18:3(6Z,9Z,12Z)/18:1(11Z)); |
| | | PC(18:3(6Z,9Z,12Z)/18:1(9Z)); |
| | | PC(18:3(9Z,12Z,15Z)/18:1(11Z)); |
| | | PC(18:3(9Z,12Z,15Z)/18:1(9Z)); |
| | | PC(18:4(6Z,9Z,12Z,15Z)/18:0); |
| | | PC(20:3(5Z,8Z,11Z)/16:1(9Z)); |
| | | PC(20:3(8Z,11Z,14Z)/16:1(9Z)); |
| | | PC(20:4(5Z,8Z,11Z,14Z)/16:0); |
| | | PC(20:4(8Z,11Z,14Z,17Z)/16:0); |
| | | PC(22:4(7Z,10Z,13Z,16Z)/14:0) |
| 783.5778 | C44H82NO8P | PC(14:1(9Z)/22:2(13Z,16Z)); |
| | | PC(16:0/20:3(5Z,8Z,11Z)); |
| | | PC(16:0/20:3(8Z,11Z,14Z)); |
| | | PC(16:1(9Z)/20:2(11Z,14Z)); |
| | | PC(18:0/18:3(6Z,9Z,12Z)); |
| | | PC(18:0/18:3(9Z,12Z,15Z)); |
| | | PC(18:1(11Z)/18:2(9Z,12Z)); |
| | | PC(18:1(9Z)/18:2(9Z,12Z)); |
| | | PC(18:2(9Z,12Z)/18:1(11Z)); |
| | | PC(18:2(9Z,12Z)/18:1(9Z)); |
| | | PC(18:3(6Z,9Z,12Z)/18:0); |
| | | PC(18:3(9Z,12Z,15Z)/18:0); |
| | | PC(20:2(11Z,14Z)/16:1(9Z)); |
| | | PC(20:3(5Z,8Z,11Z)/16:0); |
| | | PC(20:3(8Z,11Z,14Z)/16:0); |
| | | PC(22:2(13Z,16Z)/14:1(9Z)) |
| 785.5935 | C44H84NO8P | PC(18:1(9Z)/18:1(9Z)); |
| | | PC(14:0/22:2(13Z,16Z)); |
| | | PC(14:1(9Z)/22:1(13Z)); |
| | | PC(16:0/20:2(11Z,14Z)); |
| | | PC(16:1(9Z)/20:1(11Z)); PC(18:0/18:2(9Z,12Z)); |
| | | PC(18:1(11Z)/18:1(11Z)); |
| | | PC(18:1(11Z)/18:1(9Z)); |
| | | PC(18:1(9Z)/18:1(11Z)); PC(18:2(9Z,12Z)/18:0); |
| | | PC(20:1(11Z)/16:1(9Z)); |
| | | PC(20:2(11Z,14Z)/16:0); |
| | | PC(22:1(13Z)/14:1(9Z)); |
| | | PC(22:2(13Z,16Z)/14:0) |
| 786.2385 | C39H38N4O14 | Heptacarboxylporphyrin I |
| 787.6091 | C44H86NO8P | PC(14:0/22:1(13Z)); PC(14:1(9Z)/22:0); |
| | | PC(16:0/20:1(11Z)); PC(16:1(9Z)/20:0); |
| | | PC(18:0/18:1(11Z)); PC(18:0/18:1(9Z)); |
| | | PC(18:1(11Z)/18:0); PC(18:1(9Z)/18:0); |
| | | PC(20:0/16:1(9Z)); PC(20:1(11Z)/16:0); |
| | | PC(22:0/14:1(9Z)); PC(22:1(13Z)/14:0) |
| 789.5672 | C46H80NO7P | PC(20:5(5Z,8Z,11Z,14Z,17Z)/P-18:1(11Z)); |
| | | PC(20:5(5Z,8Z,11Z,14Z,17Z)/P-18:1(9Z)); |
| | | PC(22:6(4Z,7Z,10Z,13Z,16Z,19Z)/P-16:0); |
| | | PC(P-16:0/22:6(4Z,7Z,10Z,13Z,16Z,19Z)); |
| | | PC(P-18:1(11Z)/20:5(5Z,8Z,11Z,14Z,17Z)); |
| | | PC(P-18:1(9Z)/20:5(5Z,8Z,11Z,14Z,17Z)) |
| 789.6248 | C44H88NO8P | PC(14:0/22:0) |
| 791.5829 | C46H82NO7P | PC(20:4(5Z,8Z,11Z,14Z)/P-18:1(11Z)); |
| | | PC(20:4(5Z,8Z,11Z,14Z)/P-18:1(9Z)); |
| | | PC(20:4(8Z,11Z,14Z,17Z)/P-18:1(11Z)); |
| | | PC(20:4(8Z,11Z,14Z,17Z)/P-18:1(9Z)); |
| | | PC(20:5(5Z,8Z,11Z,14Z,17Z)/P-18:0); |
| | | PC(22:5(4Z,7Z,10Z,13Z,16Z)/P-16:0); |
| | | PC(22:5(7Z,10Z,13Z,16Z,19Z)/P-16:0); PC(P- |
| | | 16:0/22:5(4Z,7Z,10Z,13Z,16Z)); PC(P- |
| | | 18:0/20:5(5Z,8Z,11Z,14Z,17Z)); PC(P- |
| | | 18:1(11Z)/20:4(5Z,8Z,11Z,14Z)); |
| | | PC(dm18:1(11Z)/20:4(8Z,11Z,14Z,17Z)); PC(P- |
| | | 18:1(9Z)/20:4(5Z,8Z,11Z,14Z)); PC(P- |
| | | 18:1(9Z)/20:4(8Z,11Z,14Z,17Z)); PC(o- |
| | | 16:0/22:6(4Z,7Z,10Z,13Z,16Z,19Z)) |
| 795.6142 | C46H86NO7P | PC(20:2(11Z,14Z)/P-18:1(11Z)); |
| | | PC(20:2(11Z,14Z)/P-18:1(9Z)); |
| | | PC(20:3(5Z,8Z,11Z)/P-18:0); |
| | | PC(20:3(8Z,11Z,14Z)/P-18:0); PC(P- |
| | | 18:0/20:3(5Z,8Z,11Z)); PC(P- |
| | | 18:0/20:3(8Z,11Z,14Z)); PC(P- |
| | | 18:1(11Z)/20:2(11Z,14Z)); PC(P- |
| | | 18:1(9Z)/20:2(11Z,14Z)); PC(o- |
| | | 18:0/20:4(8Z,11Z,14Z,17Z)) |
| 798.6251 | C45H87N2O7P | SM(d18:0/22:2(13Z,16Z)(OH)) |
| 800.6407 | C45H89N2O7P | SM(d18:0/22:1(13Z)(OH)) |
| 801.6611 | C46H92NO7P | PC(o-16:1(9Z)/22:0) |
| 803.6768 | C46H94NO7P | PC(o-16:0/22:0) |
| 805.5622 | C46H80NO8P | PC(16:0/22:6(4Z,7Z,10Z,13Z,16Z,19Z)); |
| | | PC(16:1(9Z)/22:5(4Z,7Z,10Z,13Z,16Z)); |
| | | PC(16:1(9Z)/22:5(7Z,10Z,13Z,16Z,19Z)); |
| | | PC(18:1(11Z)/20:5(5Z,8Z,11Z,14Z,17Z)); |
| | | PC(18:1(9Z)/20:5(5Z,8Z,11Z,14Z,17Z)); |
| | | PC(18:2(9Z,12Z)/20:4(5Z,8Z,11Z,14Z)); |
| | | PC(18:2(9Z,12Z)/20:4(8Z,11Z,14Z,17Z)); |
| | | PC(18:3(6Z,9Z,12Z)/20:3(5Z,8Z,11Z)); |
| | | PC(18:3(6Z,9Z,12Z)/20:3(8Z,11Z,14Z)); |
| | | PC(18:3(9Z,12Z,15Z)/20:3(5Z,8Z,11Z)); |
| | | PC(18:3(9Z,12Z,15Z)/20:3(8Z,11Z,14Z)); |
| | | PC(18:4(6Z,9Z,12Z,15Z)/20:2(11Z,14Z)); |
| | | PC(20:2(11Z,14Z)/18:4(6Z,9Z,12Z,15Z)); |
| | | PC(20:3(5Z,8Z,11Z)/18:3(6Z,9Z,12Z)); |
| | | PC(20:3(5Z,8Z,11Z)/18:3(9Z,12Z,15Z)); |
| | | PC(20:3(8Z,11Z,14Z)/18:3(6Z,9Z,12Z)); |
| | | PC(20:3(8Z,11Z,14Z)/18:3(9Z,12Z,15Z)); |
| | | PC(20:4(5Z,8Z,11Z,14Z)/18:2(9Z,12Z)); |
| | | PC(20:4(8Z,11Z,14Z,17Z)/18:2(9Z,12Z)); |
| | | PC(20:5(5Z,8Z,11Z,14Z,17Z)/18:1(11Z)); |
| | | PC(20:5(5Z,8Z,11Z,14Z,17Z)/18:1(9Z)); |
| | | PC(22:5(4Z,7Z,10Z,13Z,16Z)/16:1(9Z)); |
| | | PC(22:5(7Z,10Z,13Z,16Z,19Z)/16:1(9Z)); |
| | | PC(22:6(4Z,7Z,10Z,13Z,16Z,19Z)/16:0) |
| 807.5778 | C46H82NO8P | PC(16:0/22:5(4Z,7Z,10Z,13Z,16Z)); |
| | | PC(16:0/22:5(7Z,10Z,13Z,16Z,19Z)); |
| | | PC(16:1(9Z)/22:4(7Z,10Z,13Z,16Z)); |
| | | PC(18:0/20:5(5Z,8Z,11Z,14Z,17Z)); |
| | | PC(18:1(11Z)/20:4(5Z,8Z,11Z,14Z)); |
| | | PC(18:1(11Z)/20:4(8Z,11Z,14Z,17Z)); |
| | | PC(18:1(9Z)/20:4(5Z,8Z,11Z,14Z)); |
| | | PC(18:1(9Z)/20:4(8Z,11Z,14Z,17Z)); |
| | | PC(18:2(9Z,12Z)/20:3(5Z,8Z,11Z)); |
| | | PC(18:2(9Z,12Z)/20:3(8Z,11Z,14Z)); |
| | | PC(18:3(6Z,9Z,12Z)/20:2(11Z,14Z)); |
| | | PC(18:3(9Z,12Z,15Z)/20:2(11Z,14Z)); |
| | | PC(18:4(6Z,9Z,12Z,15Z)/20:1(11Z)); |
| | | PC(20:1(11Z)/18:4(6Z,9Z,12Z,15Z)); |
| | | PC(20:2(11Z,14Z)/18:3(6Z,9Z,12Z)); |
| | | PC(20:2(11Z,14Z)/18:3(9Z,12Z,15Z)); |
| | | PC(20:3(5Z,8Z,11Z)/18:2(9Z,12Z)); |
| | | PC(20:3(8Z,11Z,14Z)/18:2(9Z,12Z)); |
| | | PC(20:4(5Z,8Z,11Z,14Z)/18:1(11Z)); |
| | | PC(20:4(5Z,8Z,11Z,14Z)/18:1(9Z)); |
| | | PC(20:4(8Z,11Z,14Z,17Z)/18:1(11Z)); |
| | | PC(20:4(8Z,11Z,14Z,17Z)/18:1(9Z)); |
| | | PC(20:5(5Z,8Z,11Z,14Z,17Z)/18:0); |
| | | PC(22:4(7Z,10Z,13Z,16Z)/16:1(9Z)); |
| | | PC(22:5(4Z,7Z,10Z,13Z,16Z)/16:0); |
| | | PC(22:5(7Z,10Z,13Z,16Z,19Z)/16:0) |
| 809.5935 | C46H84NO8P | PC(16:0/22:4(7Z,10Z,13Z,16Z)); |
| | | PC(18:0/20:4(5Z,8Z,11Z,14Z)); |
| | | PC(18:0/20:4(8Z,11Z,14Z,17Z)); |
| | | PC(18:1(11Z)/20:3(5Z,8Z,11Z)); |
| | | PC(18:1(11Z)/20:3(8Z,11Z,14Z)); |
| | | PC(18:1(9Z)/20:3(5Z,8Z,11Z)); |
| | | PC(18:1(9Z)/20:3(8Z,11Z,14Z)); |
| | | PC(18:2(9Z,12Z)/20:2(11Z,14Z)); |
| | | PC(18:3(6Z,9Z,12Z)/20:1(11Z)); |
| | | PC(18:3(9Z,12Z,15Z)/20:1(11Z)); |
| | | PC(18:4(6Z,9Z,12Z,15Z)/20:0); |
| | | PC(20:0/18:4(6Z,9Z,12Z,15Z)); |
| | | PC(20:1(11Z)/18:3(6Z,9Z,12Z)); |
| | | PC(20:1(11Z)/18:3(9Z,12Z,15Z)); |
| | | PC(20:2(11Z,14Z)/18:2(9Z,12Z)); |
| | | PC(20:3(5Z,8Z,11Z)/18:1(11Z)); |
| | | PC(20:3(5Z,8Z,11Z)/18:1(9Z)); |
| | | PC(20:3(8Z,11Z,14Z)/18:1(11Z)); |
| | | PC(20:3(8Z,11Z,14Z)/18:1(9Z)); |
| | | PC(20:4(5Z,8Z,11Z,14Z)/18:0); |
| | | PC(20:4(8Z,11Z,14Z,17Z)/18:0); |
| | | PC(22:4(7Z,10Z,13Z,16Z)/16:0) |
| 811.6091 | C46H86NO8P | PC(16:1(9Z)/22:2(13Z,16Z)); |
| | | PC(18:0/20:3(5Z,8Z,11Z)); |
| | | PC(18:0/20:3(8Z,11Z,14Z)); |
| | | PC(18:1(11Z)/20:2(11Z,14Z)); |
| | | PC(18:1(9Z)/20:2(11Z,14Z)); |
| | | PC(18:2(9Z,12Z)/20:1(11Z)); |
| | | PC(18:3(6Z,9Z,12Z)/20:0); |
| | | PC(18:3(9Z,12Z,15Z)/20:0); |
| | | PC(20:0/18:3(6Z,9Z,12Z)); |
| | | PC(20:0/18:3(9Z,12Z,15Z)); |
| | | PC(20:1(11Z)/18:2(9Z,12Z)); |
| | | PC(20:2(11Z,14Z)/18:1(11Z)); |
| | | PC(20:2(11Z,14Z)/18:1(9Z)); |
| | | PC(20:3(5Z,8Z,11Z)/18:0); |
| | | PC(20:3(8Z,11Z,14Z)/18:0); |
| | | PC(22:2(13Z,16Z)/16:1(9Z)) |
| 812.6771 | C47H93N2O6P | SM(d18:1/24:1(15Z)) |
| 814.6928 | C47H95N2O6P | SM(d18:0/24:1(15Z)) |
| 815.6404 | C46H90NO8P | PC(14:0/24:1(15Z)) |
| 815.7006 | C47H96N2O6P | SM(d18:1/24:0) |
| 816.7084 | C47H97N2O6P | SM(d18:0/24:0) |
| 817.6561 | C46H92NO8P | PC(14:0/24:0) |
| 819.6142 | C48H86NO7P | PC(o-18:0/22:6(4Z,7Z,10Z,13Z,16Z,19Z)) |
| 821.6298 | C48H88NO7P | PC(o-20:1(11Z)/20:4(8Z,11Z,14Z,17Z)) |
| 823.6455 | C48H90NO7P | PC(22:2(13Z,16Z)/P-18:1(11Z)); |
| | | PC(22:2(13Z,16Z)/P-18:1(9Z)); PC(P- |
| | | 18:1(11Z)/22:2(13Z,16Z)); PC(P- |
| | | 18:1(9Z)/22:2(13Z,16Z)); PC(o- |
| | | 20:0/20:4(8Z,11Z,14Z,17Z)) |
| 824.3831 | C41H60O17 | (2b,3b)-Dihydroxy-30-nor-12,20(29)- |
| | | oleanadiene-28-glucopyranosyloxy-23-oic acid 3- |
| | | glucuronide |
| 824.4194 | C42H64O16 | Quillaic acid 3-[galactosyl-(1->2)-glucuronide] |
| 825.6611 | C48H92NO7P | PC(o-22:0/18:3(6Z,9Z,12Z)); PC(o- |
| | | 22:0/18:3(9Z,12Z,15Z)) |
| 826.8221 | C21H20I3NO10 | Triiodothyronine glucuronide |
| 827.6768 | C48H94NO7P | PC(o-18:2(9Z,12Z)/22:0) |
| 828.6720 | C47H93N2O7P | SM(d18:0/24:1(15Z)(OH)) |
| 829.6924 | C48H96NO7P | PC(o-18:1(9Z)/22:0) |
| 830.2283 | C40H38N4O16 | Uroporphyrin III; Uroporphyrin I |
| 833.5935 | C48H84NO8P | PC(18:0/22:6(4Z,7Z,10Z,13Z,16Z,19Z)) |
| 835.6091 | C48H86NO8P | PC(18:0/22:5(4Z,7Z,10Z,13Z,16Z)) |
| 839.6404 | C48H90NO8P | PC(18:1(11Z)/22:2(13Z,16Z)); |
| | | PC(18:1(9Z)/22:2(13Z,16Z)); |
| | | PC(18:2(9Z,12Z)/22:1(13Z)); |
| | | PC(18:3(6Z,9Z,12Z)/22:0); |
| | | PC(18:3(9Z,12Z,15Z)/22:0); |
| | | PC(20:0/20:3(5Z,8Z,11Z)); |
| | | PC(20:0/20:3(8Z,11Z,14Z)); |
| | | PC(20:1(11Z)/20:2(11Z,14Z)); |
| | | PC(20:2(11Z,14Z)/20:1(11Z)); |
| | | PC(20:3(5Z,8Z,11Z)/20:0); |
| | | PC(20:3(8Z,11Z,14Z)/20:0); |
| | | PC(22:0/18:3(6Z,9Z,12Z)); |
| | | PC(22:0/18:3(9Z,12Z,15Z)); |
| | | PC(22:1(13Z)/18:2(9Z,12Z)); |
| | | PC(22:2(13Z,16Z)/18:1(11Z)); |
| | | PC(22:2(13Z,16Z)/18:1(9Z)) |
| 840.7084 | C49H97N2O6P | SM(d18:0/26:1(17Z)) |
| 841.6561 | C48H92NO8P | PC(16:1(9Z)/24:1(15Z)) |
| 843.7319 | C49H100N2O6P | SM(d18:1/26:0) |
| 849.6611 | C50H92NO7P | PC(o-22:1(13Z)/20:4(8Z,11Z,14Z,17Z)) |
| 851.6768 | C50H94NO7P | PC(o-22:0/20:4(8Z,11Z,14Z,17Z)) |
| 853.6924 | C50H96NO7P | PC(24:1(15Z)/P-18:1(11Z)); PC(24:1(15Z)/P- |
| | | 18:1(9Z)); PC(P-18:1(11Z)/24:1(15Z)); PC(P- |
| | | 18:1(9Z)/24:1(15Z)); PC(o- |
| | | 24:0/18:3(6Z,9Z,12Z)); PC(o- |
| | | 24:0/18:3(9Z,12Z,15Z)) |
| 855.7081 | C50H98NO7P | PC(o-18:2(9Z,12Z)/24:0) |
| 856.6435 | C15H11I4NO7S | Thyroxine sulfate |
| 857.7237 | C50H100NO7P | PC(o-18:1(9Z)/24:0) |
| 861.6248 | C50H88NO8P | PC(20:0/22:6(4Z,7Z,10Z,13Z,16Z,19Z)) |
| 863.6404 | C50H90NO8P | PC(18:4(6Z,9Z,12Z,15Z)/24:1(15Z)) |
| 865.6561 | C50H92NO8P | PC(18:3(6Z,9Z,12Z)/24:1(15Z)); |
| | | PC(18:3(9Z,12Z,15Z)/24:1(15Z)); |
| | | PC(18:4(6Z,9Z,12Z,15Z)/24:0); |
| | | PC(20:0/22:4(7Z,10Z,13Z,16Z)); |
| | | PC(20:2(11Z,14Z)/22:2(13Z,16Z)); |
| | | PC(20:3(5Z,8Z,11Z)/22:1(13Z)); |
| | | PC(20:3(8Z,11Z,14Z)/22:1(13Z)); |
| | | PC(20:4(5Z,8Z,11Z,14Z)/22:0); |
| | | PC(20:4(8Z,11Z,14Z,17Z)/22:0); |
| | | PC(22:0/20:4(5Z,8Z,11Z,14Z)); |
| | | PC(22:0/20:4(8Z,11Z,14Z,17Z)); |
| | | PC(22:1(13Z)/20:3(5Z,8Z,11Z)); |
| | | PC(22:1(13Z)/20:3(8Z,11Z,14Z)); |
| | | PC(22:2(13Z,16Z)/20:2(11Z,14Z)); |
| | | PC(22:4(7Z,10Z,13Z,16Z)/20:0); |
| | | PC(24:0/18:4(6Z,9Z,12Z,15Z)); |
| | | PC(24:1(15Z)/18:3(6Z,9Z,12Z)); |
| | | PC(24:1(15Z)/18:3(9Z,12Z,15Z)) |
| 869.6874 | C50H96NO8P | PC(18:1(11Z)/24:1(15Z)); |
| | | PC(18:1(9Z)/24:1(15Z)); PC(18:2(9Z,12Z)/24:0); |
| | | PC(20:0/22:2(13Z,16Z)); |
| | | PC(20:1(11Z)/22:1(13Z)); |
| | | PC(20:2(11Z,14Z)/22:0); |
| | | PC(22:0/20:2(11Z,14Z)); |
| | | PC(22:1(13Z)/20:1(11Z)); |
| | | PC(22:2(13Z,16Z)/20:0); |
| | | PC(24:0/18:2(9Z,12Z)); |
| | | PC(24:1(15Z)/18:1(11Z)); |
| | | PC(24:1(15Z)/18:1(9Z)) |
| 871.7030 | C50H98NO8P | PC(18:0/24:1(15Z)) |
| 873.7187 | C50H100NO8P | PC(18:0/24:0) |
| 875.6768 | C52H94NO7P | PC(o-22:0/22:6(4Z,7Z,10Z,13Z,16Z,19Z)) |
| 877.6924 | C52H96NO7P | PC(o-22:2(13Z,16Z)/22:3(10Z,13Z,16Z)) |
| 879.7081 | C52H98NO7P | PC(o-22:1(13Z)/22:3(10Z,13Z,16Z)) |
| 881.7237 | C52H100NO7P | PC(o-22:0/22:3(10Z,13Z,16Z)); PC(o- |
| | | 22:1(13Z)/22:2(13Z,16Z)) |
| 936.3277 | C45H52N4O18 | Bilirubin diglucuronide |
| 940.5032 | C48H76O18 | 28-Glucosyloleanolic acid 3-[arabinosyl-(1->2)- |
| | | 6-methylglucuronide] |
| 942.5188 | C48H78O18 | Soyasapogenol B 3-O-[a-L-rhamnosyl-(1->4)-b- |
| | | D-galactosyl-(1->4)-b-D-glucuronide] |
| 952.7188 | C21H1914NO10 | Thyroxine glucuronide |
| 956.4617 | C47H72O20 | Quillaic acid 3-[xylosyl-(1->3)-[galactosyl-(1- |
| | | >2)]-glucuronide] |
| 970.4773 | C48H74O20 | Quillaic acid 3-[rhamnosyl-(1->3)-[galactosyl-(1- |
| | | >2)]-glucuronide]; 28-Glucosyl-3b-hydroxy-12- |
| | | oleanene-30-methoxy-28-oic acid 3-[arabinosyl- |
| | | (1->3)-glucuronide] |
| 972.4930 | C48H76O20 | 28-Glucosylarjunolate 3-[rhamnosyl-(1->3)- |
| | | glucuronide] |
| 986.4723 | C48H74O21 | 28-Glucosyl-30-methyl-3b,23-dihydroxy-12- |
| | | oleanene-28,30-dioate 3-[arabinosyl-(1->3)- |
| | | glucuronide] |
| 1006.4365 | C43H66N12O12S2 | Oxytocin |
| 1023.6706 | C52H97NO18 | Trihexosylceramide (d18:1/16:0) |
| 1049.6862 | C54H99NO18 | Trihexosylceramide (d18:1/9Z-18:1) |
| 1051.7019 | C54H101NO18 | Trihexosylceramide (d18:1/18:0) |
| 1079.7332 | C56H105NO18 | Trihexosylceramide (d18:1/20:0) |
| 1088.5040 | C52H80O24 | Medicagenic acid 3-O-b-D-glucuronide 28-O-[b- |
| | | D-xylosyl-(1->4)-a-L-rhamnosyl-(1->2)-a-L- |
| | | arabinosyl] ester |
| 1102.5560 | C54H86O23 | Protoprimulagenin A 3-[glucosyl-(1->3)- |
| | | galactosyl-(1->4)-[rhamnosyl-(1->2)]- |
| | | glucuronide] |
| 1107.7645 | C58H109NO18 | Trihexosylceramide (d18:1/22:0) |
| 1163.8271 | C62H117NO18 | Trihexosylceramide (d18:1/26:1(17Z)) |

We found many substances that correlate with the human conditions we measured in these experiments that have not been reported to be products of human metabolism. These are widely abundant in food, air, water, and other sources, and metabolic information is apparently impressed on them while they are inside human tissues. We decided to limit these diagnostic calculations to those designated as human products [13], so about 700 human metabolites that passed our 80% criteria in each experiment are used herein in each diagnostic evaluation. On average, the amounts of about eight adducts and isotopes in the spectra were added together to obtain the value for each of the 700.

We have found that the logarithmic ratios of amounts of metabolites contain better diagnostic information than the absolute values, which is in accord with the general behavior of chemical systems. So, we calculated about 500,000 parameters by dividing all of the 700 amounts of metabolites with each other and then computing the logarithms. These 500,000 parameters were ordered by nonparametric correlation probability, and the most correlating unique 500 parameters were used for diagnosis. These 500 (1,000 including the inverses) included between 120 and 150 different metabolites, depending upon individual disease, and no single metabolite was present in more than 5% of the 500 selected. Although there were far more than 500 parameters with high correlation probabilities, we found that inclusion of more than 500 had little marginal diagnostic value herein. Use of ratios in this computation also removes any remaining concentration-normalizing insufficiencies.

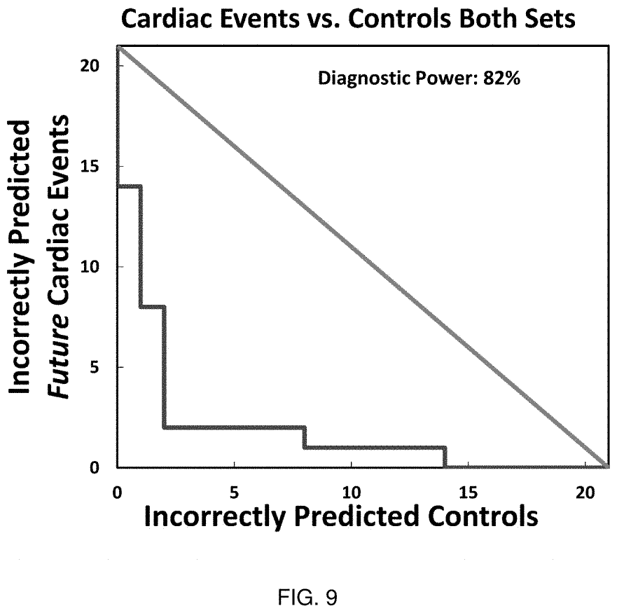

In the case of the predictive cardiac event profile, the 500 parameters included 147 human metabolic urinary substances in the first trial, 146 in the second, and 148 in the combined diagnostic power evaluation shown in FIG. 9. While sophisticated pattern recognition techniques are available, we have used a simplified procedure herein, in which diagnostic coefficients [5] are calculated.

Diagnostic coefficients RA are defined as:

$${R}_{A} = \frac{100}{{\sum }_{i = 1}^{n} {r}_{i}} [ {\sum }_{i = 1}^{n} \frac{\vert {A}_{i} - {Y}_{i} \vert {r}_{i}}{{A}_{i} + {Y}_{i}} - {\sum }_{i = 1}^{n} \frac{\vert {A}_{i} - {O}_{i} \vert {r}_{i}}{{A}_{i} + {O}_{i}} ]$$

where Ai is the normalized value of the ith parameter in the mass spectrum, A, that is being classified. Yi and Oi are the average values of the corresponding parameters in the two groups being compared, n is the number of parameters in the calculation, and ri is a weight constant that was set equal to 1 for all parameters in the calculations herein for simplicity in evaluating these results.

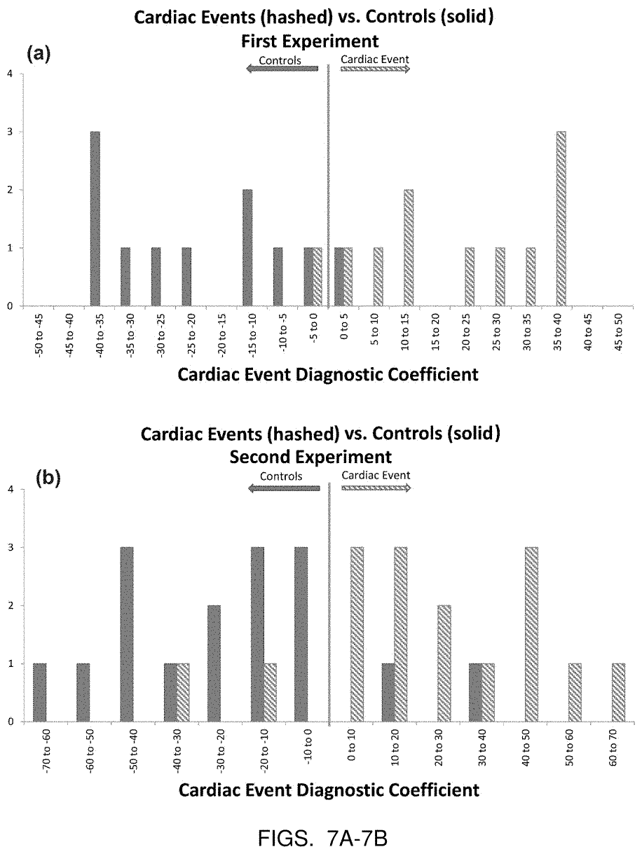

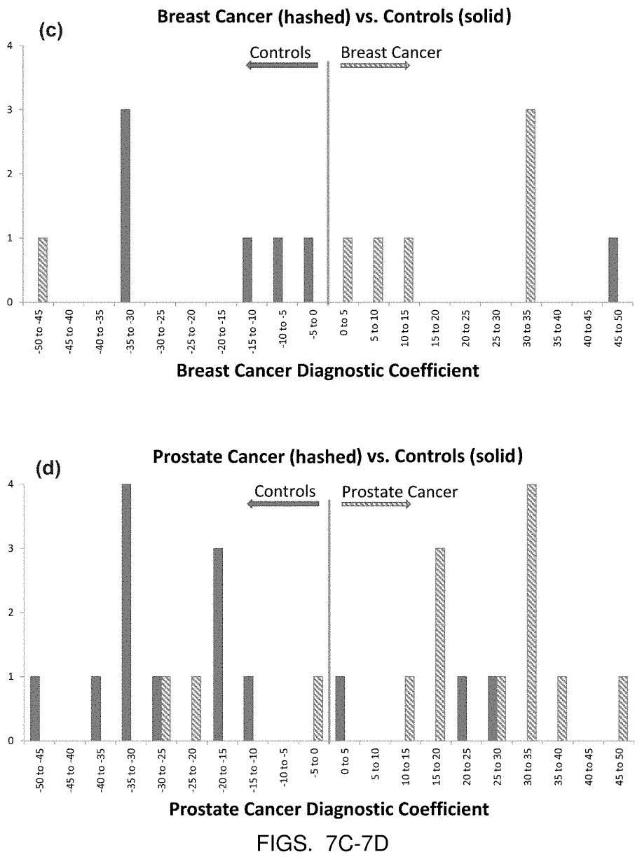

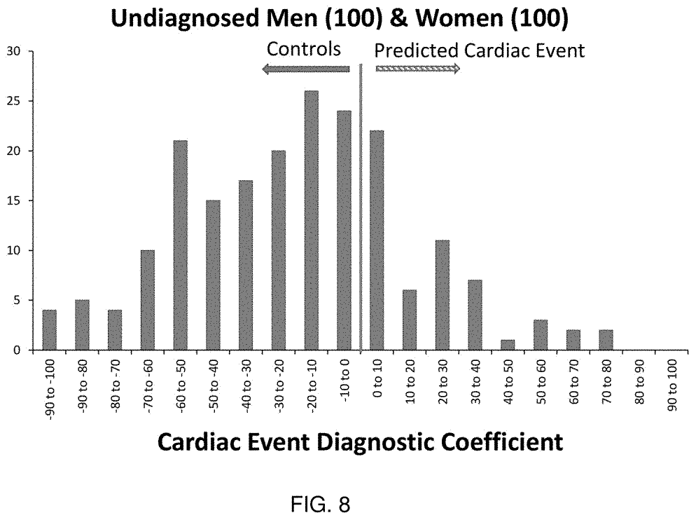

By this procedure, each parameter (logarithmic metabolite ratio) is averaged for the test group and an appropriate control group. Each subject in the disease analyses was paired with an age and sex-matched control, and diagnostic coefficients for each of the pair were computed. The pair is excluded from determination of the averages of the parameters to which it is compared, the averages being thus recomputed for each comparison. This exclusion prevents the pair from biasing the averages in its own favor. The average coefficient for the two is computed and the quantitative diagnostic coefficient deviation toward the experimental or control group averages for each determined. These deviations are plotted on a diagnostic coefficient graph as shown in FIGS. 7A-7D and 8.

To simplify comparisons in FIGS. 7A-7D and 8, these values were normalized to a range between −50 for the most extreme average of the control subjects and +50 for the most extreme average of the subjects manifesting the condition of interest.

FIGS. 7A-7D show bar graphs, which illustrate the diagnostic separations achieved. From the combined diagnostic coefficient order of the trial and control groups shown in these graphs, the nonparametric probability that a separation into two groups by metabolic profiling has been achieved is computed. For the two cardiac event analyses, the breast cancer analysis, and the prostate cancer analysis, these probabilities are 99.5%, 99.8%, 94%, and 97%, respectively. While the breast cancer separation appears better than the prostate cancer pattern, it has a lower probability. The reason for this is that fewer pairs of diseased and non-diseased subjects were used in the breast cancer analysis.

These diagnostic coefficients can then be ordered and plotted in diagnostic power graphs as illustrated for cardiac event prediction in FIG. 9. The coefficients are placed in numerical order for the subjects being evaluated, and this linear distribution is divided at all possible division points to create the diagnostic power graph. This graphing method was developed [5] to account for the fact that diagnostic profiles do not contain within themselves essential information about how they will be used, such as the tolerances for false positives and false negatives, which depend on anticipated medical or other actions.

Since cardiac events very often lead to unexpected and immediate death (9 of 21 or 43% of the urine bank volunteers suffering cardiac events in these two analyses died from the event), more false positives would be tolerable for this disease than, for example, prostate cancer. FIG. 9 shows that 19 out of 21 cardiac event-prone subjects were identified with only two apparent false positives among the normal controls. If fewer false negatives are desired the increased number of false positives is evident from the graph. For random data and no diagnostic power, the data would follow the theoretical gray line on the graph. The “diagnostic power” of 82% in FIG. 9 represents the percentage area between the random gray line and a perfect correlation of a point in the origin.

Results and Discussion

Sex and Age

FIGS. 4A-4B show the cumulative distribution function of nonparametric probability of non-correlation, P, of MRMS-measured urinary peaks with sex and age. The peaks for sex used age-matched controls, and those for age used sex-matched controls. The lower gray line in each graph is the theoretical plot for non-correlated measurements.

The cumulative distribution function of nonparametric probabilities of non-correlation with sex (FIG. 4A) shows a very strong profile, affecting more than 30% of the peaks. There are 1,000 peaks strongly correlating and 3,000 reasonably correlating, reflecting the pervasive metabolic differences between men and women. There were 100 men and 100 women with no known health problems in this evaluation. When the individual correlation probabilities of a large number of substances are calculated, these probabilities are linearly distributed between 0 and 1 if there is no overall correlation. For example, if there are 1,000 peaks, the sum of the probabilities of non-correlation at or below 0.01 will be about 10, below 0.1 about 100, below 0.2 about 200, and so on.

If, however, some of the peaks are correlated, the low probabilities are raised in number, which raises the low probability part of the line. So, for example, the sex probability distribution here is composed of an approximately linear distribution of about 5,000 non-correlated peaks and about 3,000 correlated peaks.

Statistical detection of correlation increases with the number of measurements of each substance, so there may well be far more than 3,000 peaks actually correlated, but the additional weaker correlations will not be evident unless more individual urines are analyzed.

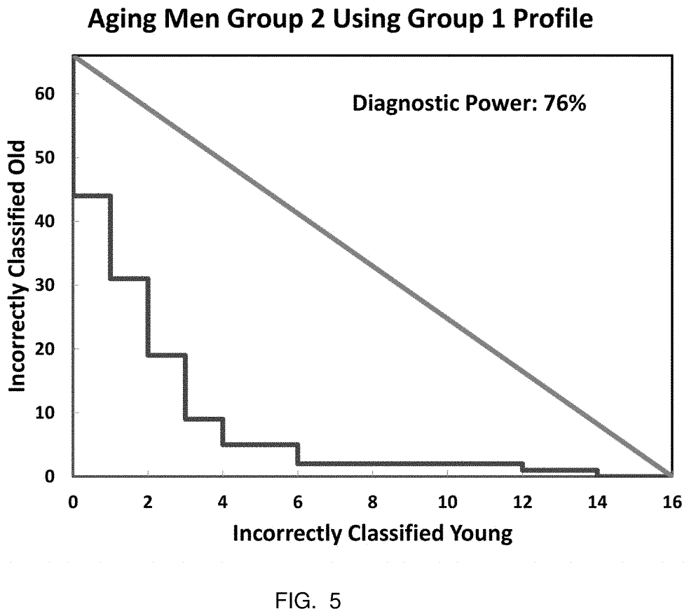

The cumulative distribution function for aging was calculated for these same men and women. The diagnostic coefficients for aging computed for this profile were calculated using half of the male subjects to establish a profile for aging (group 1) and the other half used to evaluate the profile (group 2). This revealed a diagnostic power for group 2 of 76% shown in FIG. 5.

This diagnostic power is below 100% partly because the separations are by chronological age, while the measurements are of physiological age. As more data on medical histories and lifespan accumulates over time, the metabolic profile will give an increasingly accurate estimate of a subject's position on an axis of physiological aging and thus of years remaining in the individual's lifespan.

Also, since the statistical years of life remaining to these younger and older men overlap, a complete separation and diagnostic power of 100% is not possible. This has been discussed more completely elsewhere [8].

Thus, the urine bank and profiling analysis will eventually reveal the statistically estimated years of life remaining for these volunteers. Moreover, since all samples are stored at −80° C. and analytical technology will continue to improve, more accurate measurements of more substances will become available to refine this profile.

The aging profile herein shows about 30% of the peaks correlate with age. This is consistent with the approximately 30% of substances found to be age correlated in the 1970s [8], with far fewer substances.

During the 1970s research wherein age-dependent metabolic profiles were first observed, most metabolites were not identifiable by the chromatographic techniques utilized. Among 20 that were identified were aspartic acid, glutathione, cystine, alpha-amino butyric acid, and glutamic acid, which increased with age, and histidine, asparagine+glutamine, serine, glycine, threonine, alpha-amino adipic acid, alanine, lysine, valine, ethanolamine, and taurine, which decreased with age [8]. All 16 of these deviated with age in the same directions in the MRMS analyses reported herein as they did in the 1970s research. The other 4 substances identified in the 1970s did not deviate in the same directions, but these were present in very small amounts and therefore subject to high experimental error.

These results illustrate a characteristic of quantitative metabolic profiling in that the urinary amounts of thousands of compounds are useful for profiling, even though they would not necessarily be expected to be especially biochemically relevant. Biochemical interconnectedness in human metabolism induces weak correlations into thousands of molecular species, and these can be statistically summed to provide practical diagnostic value.

For example, it was found in the 1970s [12] that urinary amines and amino acids were highly correlated with sex, and we have replicated this finding here, even though specific biochemical links of these substances to human sex are generally unknown.

The physiological age profile should reflect the probable years of life remaining as a result of physiological deterioration and increased susceptibility to disease, especially to life-threatening illnesses. Quantitative metabolic profiling of physiological age should eventually permit useful experiments to be performed on populations and on individuals with respect to the effects of diet, exercise, chemical supplements, and other lifestyle-adjustable parameters.

Cardiac Events and Breast and Prostate Cancer Analyses

The cumulative distribution functions of nonparametric non-correlation probabilities for cardiac events, breast cancer, and prostate cancer were determined as shown in FIGS. 6A-6C. The urine samples were provided by the volunteers and stored in the urine bank 4 to 30 months before these illnesses were symptomatically experienced by the volunteers and medically diagnosed, with the exception that 5 volunteers of the 11 in the first cardiac-event group had also experienced earlier heart health problems.

At P=0.1 and based on our experimentally determined σ for the gray (lower) line, the black (upper) lines obtained from our analyses differ from the gray (lower) lines by 5.5σ for the first cardiac event profile, 2.2σ for the breast cancer profile, and 0.4σ for the prostate cancer profile.

There is, therefore, a greater than 99.99% probability that a cardiac event profile has been detected and a greater than 95% probability that a breast cancer profile has been detected.

There are fewer unique cumulative probabilities plotted for breast cancer as a result of the smaller number of subjects diagnosed with breast cancer, and therefore it exhibits a more broken black line.

The relatively strong cardiac event profile might be anticipated because a deteriorating heart would be expected to have especially widespread consequences in metabolic processes. To confirm the first cardiac event profile, we performed a second analysis with 16 volunteer subjects, none of whom were known to have ever experienced a heart problem prior to providing the analyzed urine sample, but all of whom suffered a cardiac event in the 4 to 30-month period following deposit of the sample. The result is shown in FIG. 7B.

In this analysis, an improved version of the Bruker FTICR-MS with greater sensitivity was utilized; the mass range was 75 to 1,000; 1.5 second transients were collected; and 300 transients were averaged.

Cardiac Event Prediction

The cardiac event samples analyzed herein were given by the volunteers before they suffered symptoms and were diagnosed with cardiac disease (except for 5 samples in the first cardiac event analysis).

A correlation was qualitatively observed wherein the cardiac event diagnostic coefficient apparently became larger as the time of the cardiac event approached, but the small size of these sample sets prevents corroboration of this observation with statistical reliability.

Of the 21 cardiac event subjects in FIGS. 7A-7D, the time between the analyzed sample and the cardiac event was between 4 and 11 months for 11 subjects, 14 and 22 months for 9 subjects, and 30 for one subject.

We next evaluated whether these profiles were strong enough for predictive and possibly preventive use. To evaluate this, diagnostic coefficients were calculated for the disease victims and their individual sex- and age-matched controls as shown in FIGS. 7A and 7B. The crosshatched bars are those who suffered cardiac events after providing the samples and the black bars are controls.

The numerical distributions in these disease diagnostic coefficient values shown in FIGS. 7A-7D provide nonparametric probabilities that these measured profiles are diagnostic of the diseases prior to the later symptoms and medical diagnoses. These probabilities are 99.5% and 99.8% for the two separate cardiac event profile analyses.

The cardiac event prediction profile, discovered in the first set of subjects and confirmed in the second, is especially remarkable.

Diagnostic coefficients were also calculated for the cardiac event profile of the 100 men and 100 women whose samples were used in the age and sex analyses, as shown in FIG. 8.

Those among the 200 with a positive heart disease diagnostic coefficient comprise 28% of the group, while CDC (Centers for Disease Control and Prevention) statistics indicate that about 27% of individuals in this age distribution are expected to eventually die from heart disease.

The reliability of this percentage-of-population finding of 28% is enhanced as compared with individual diagnosis, since analytical profiling experimental noise is averaged over 200 analyses in the result.

These results demonstrate that there is a metabolic profile present in the urine of people who have not yet experienced cardiac events, which is likely to be of value in warning such people of this vulnerability.

The diagnostic power graph in FIG. 9 created from the ordered diagnostic coefficients of the 21 cardiac event victims and 21 age and sex-matched controls in the two cardiac event analyses combined (with averaged coefficients from these two analyses used for the 11 people in both analyses) has a diagnostic power of 82%.

If these people could have received a notification or warning as a result of their metabolic profiling, they could have sought medical help, made changes in their lives in hopes of diminishing their cardiac event probability, and taken precautions, such as equipping themselves or their associates with portable defibrillators.

Thus, we see in FIG. 9 that, if this profile had been used to inform those whose urine was analyzed, 19 of the 21 who were at risk could have been warned if the cutoff criteria had been set to a level that warned only 2 who had not suffered an event. Given the prevalence of heart disease, it is probable that several of the “control” subjects in this study will also eventually experience cardiac events, so the diagnostic power may be higher than 82%. This diagnostic power will improve when constructed with many more samples and subjects.

FIG. 9 demonstrates the value of the graphical diagnostic power evaluation [5] because the results of a quantitative profiling study do not contain information about how the profile will be used. Since cardiac events very often lead to unexpected and immediate death, more false positives would be tolerable for this disease than, for example, prostate cancer.

The metabolic profiles for these three conditions (cardiac event, breast cancer, and prostate cancer) appeared before symptoms and medical diagnosis and are unique. Each of the three profiles, when applied to the profiles of subjects with the other two diagnoses, showed no diagnostic value whatever.

Conclusions

This example demonstrates that magnetic resonance mass spectrometry (MRMS), when combined with the information in a human urine bank for calibration, can be used as a method for the empirical quantitative metabolic profiling of human health.

The empirical use of high-resolution mass spectrometry and careful sampling as illustrated in this example can make important contributions to the quality and length of human life. For example, a sample kit comprising a suitable disposable laser desorption target (on a substrate such as one of various forms of paper) on which the user places a drop of urine, allows it to dry, and then mails the target in an ordinary envelope via USPS First Class mail to a central mass spectrometry laboratory. The user could receive by e-mail, or download from an Internet web site, a coded confidential report with valuable health information for a total cost of perhaps $5, including kit, postage, and automated analysis, within a few days. Also, receiving the analysis itself, the user could submit his analysis to a statistical evaluation Internet provider of his choice. In this way, mass spectrometric technology could make valuable information for preventive, diagnostic, and therapeutic medicine immediately available to all people, regardless of their social and economic circumstances.

### REFERENCES

1. Marshall A G, Chen T. 40 years of Fourier transform ion cyclotron resonance mass spectrometry. Int J Mass Spectrom 2015; 377:410-420.
2. Fenn J B, Mann M, Meng C K, Wong S F, Whitehouse C M. electrospray ionization for mass spectrometry of large biomolecules. Science 1989; 246:64-71.
3. Tanaka K, Waki H, Ido Y, et al. Protein and polymer analyses up to 100,000 by laser ionization time-of-flight mass spectrometry. Rapid Commun Mass Spectrom 1988; 2:151-153.
4. Karas M, Bachmann D, Hillenkamp F influence of wavelength in high irradiance ultraviolet laser desorption mass spectrometry of organic molecules. Anal Chem 1985; 57:2935-2939.
5. Robinson N E, Robinson A B. Origins of metabolic profiling. In: Metz T O, ed. Metabolic Profiling: Methods and Protocols (Methods in Molecular Biology 708). Springer Protocols, Humana Press; 2011:1-24.
6. Robinson A B, Pauling L C. Techniques of orthomolecular diagnosis. Clin Chem 1974; 20:961-965.
7. Robinson A B, Dirren H, Sheets A, Miguel J, Lundgren P R. Quantitative aging pattern in mouse urine vapor as measured by gas-liquid chromatography. Exp Gerontol 1976; 11:11-16.
8. Robinson A B, Robinson L R. Quantitative measurement of human physiological age by profiling of body fluids and pattern recognition. Mech Ageing Dev 1991; 59:47-67.
9. Lei Z, Huhman D V, Sumner L V. Mass spectrometry strategies in metabolomics. J Biol Chem 2011; 286:25435-25442.
10. Brown S C, Kruppa G, Dasseux J L. Metabolomics applications of FT-ICR mass spectrometry. Mass Spectrom Rev 2005; 24:223-231.
11. Alder H L, Roessler E B. Introduction to Probability and Statistics. 6th ed. WH Freeman; 1977:192-212.
12. Dirren H, Robinson A B, Pauling L C. Sex-related patterns in the profiles of human urinary amino acids. Clin Chem 1975; 21:1970-1975.
13. Bouatra S, Mandal R, Guo A C, et al. In: The Human Urine Metabolome. Metabolomics Innovation Centre; 2013.

### 6.2 Example 2: Magnetic Resonance Mass Spectrometry Applied to Metabolic Profiling

Abstract

Magnetic Resonance Mass Spectrometry, MRMS, based on Fourier Transform Ion Cyclotron Resonance Mass Spectrometry [1], provides unparalleled resolution, sensitivity, and mass accuracy for the measurement of substances in biological fluids. It also makes possible the measurement of very large numbers of substances in a single analysis. Thus, this technique is ideal for quantitative metabolic profiling [2-5]. We previously used MRMS for the discovery of diagnostically-useful metabolic profiles for human age, sex, prostate cancer, breast cancer, and cardiac illness in human urine obtained from a 5,000-person human urine bank [6]. These profiles were discovered in individuals who had not yet exhibited symptoms or been diagnosed with these three diseases, but who subsequently fell ill. To make these measurements with MRMS, appropriate protocols and software were developed. These developments permit the detection of more than 200,000 mass spectrometric peaks with specific molecular masses in the mass spectrum of a 5 μl urine sample by means of one 7-minute run using positive ion mode. Of these 200,000, about 30,000 are detected in analytically significant amounts. On average, each unique urinary substance is represented by 8 different masses in the 30,000, due to isotopic peaks and various salt adducts. Summing these peaks provides quantitative measurements for about 4,000 substances. Of these, we found 2,300 that are listed in a recent compilation of 2,700 human urinary substances, of which 917 are, or have been inferred to be, of human metabolic origin [7]. Of these 917, we found 833 in 80% or more of the measured urine samples and used these for diagnostic purposes [6].

Introduction

Advances in mass spectrometry, nuclear magnetic resonance, chromatography, and combinations of these techniques with each other and with other improved analytical disciplines, as applied to the study of small molecule metabolic products in living systems, have made possible such great advances in biochemistry during the past two decades that this combination of techniques and the resulting new biochemical knowledge is currently summarized under the interdisciplinary name of “metabolomics” [8].

Like other modern endeavors, this combination of technological advance and the microprocessor-Internet revolution has expected and unexpected consequences. The cornucopia of substances that can be precisely measured in a single analytical procedure and thereby made available for metabolic profiling enables revolutionary advances in preventive, diagnostic, and therapeutic medicine that were heretofore impossible. We describe herein a method for metabolic profiling that we are using to advance preventive, diagnostic, and therapeutic medicine.

MRMS exhibits extraordinary mass resolving power, mass measurement accuracy and sensitivity. It is limited, however, by the number of molecules that can be accommodated at one time in the detection cell and by charge competition in the source. Thus, it is necessary that, as much as possible, molecules extraneous to the measurement be excluded. Therefore, specific consideration was given to the way in which the samples were introduced and ionized to mitigate the presence of extraneous compounds.

Ordinarily, this limitation is avoided by separation methods, such as liquid chromatographic purification that can occur during or before sample introduction. In metabolic profiling of urine for practical diagnostic use, however, liquid chromatography adds time and expense, which limits the range of substances measured and its low-cost applicability. To avoid this time and expense, the methodology needs to deliver efficiently to the detection cell an unaltered representative set of urinary substances, with minimal inclusion of substances not present in the urine sample. Therefore, the elimination of a sample ‘clean-up’ step is preferred for this work.

Regarding ionization, atmospheric pressure ionization methods such as electrospray ionization [9], can also introduce extraneous substances which often bind to the walls of delivery tubing and the analytical capillary, thus both adding and/or subtracting substances to and from the analytical mixture. Alternatively, matrix assisted laser desorption ionization (MALDI) [10-11], was considered as it eliminates the issues relating to carry-over, sample absorption, and contamination. MALDI involves embedding the sample in a chemical matrix, which aids in homogeneous ionization, but the matrix chemicals, even those available with the highest purity, contain large numbers of contaminants in amounts comparable to the substances in the urine samples. This problem is avoided altogether by using laser desorption ionization (LDI), wherein the sample is introduced and directly ionized without passing by absorptive surfaces. For this work, LDI from nanopost array ionization plates was used which, although providing some contamination is much less so than the conventional matrix chemicals.

Additionally, there is the challenge of salts, especially sodium and potassium, that are present in urine in large amounts, which complicate the mass spectra and diminish uniform ionization. Desalting the urine, however, introduces substances from the desalting column, and urinary substances are often lost by retention on the desalting column. This can be avoided by not desalting the samples prior to analysis. The resulting complications in ionization can be partially corrected by using unusually high laser intensity in the measurements.

Ordinary mass spectra contain multiple forms of the measured species, including isotope peaks and adducts with other species, Without desalting, this is substantially exacerbated by salt adducts of the measured species. The extraordinary resolution of the MRMS method, however, permits separate measurement of most of these adducts, even in the complex mixture of urine metabolites. Thus, we have developed software that enables identification of the isotopic peaks of the molecular ion and all adducts and sums them to provide the measured amounts of the desired urinary species.

In the resulting urine analyses of positive ions, about 8 peaks from isotopes and different adduct forms are present, on average, in analytically significant quantity for each urinary species. Our current addition procedure neglects the fact that different adducts may have different quantitative ionization characteristics. This can be corrected by using internal calibrants.

Therefore, the only sample manipulation used herein was dilution of the samples with ultra-pure water to provide comparable urinary concentrations of the measured molecular species.

With contamination us reduced by using nanopost LDI without a chemical matrix and analysis of samples that were not desalted or otherwise manipulated in ways that could introduce contamination or remove urinary substances, very useful urinary profiles emerge from the analyses.

Even with these precautions, the urine analyses may still show peaks from contaminants, but this was reduced to a usable level.

It is estimated that the MRMS detection cell accommodates about 1 million charges during the measurement of each transient, without degradation in resolution or mass accuracy. By using 500 laser shots to produce each transient and averaging 300 transients from different positions on the nanopost plate as provided by Bruker automation, we are measuring an estimated 300 million molecules in each analysis, since the ions measured in these experiments are singly charged.

We observe approximately 30,000 peaks of analytically significant size distributed over a range of about 3 orders of magnitude. This is an estimated average of about 10,000 molecules per peak. So, substances that we utilize in the lower part of this range may contain as few as 1,000 molecules. Thus, there is a need for ultra-clean sample manipulation.

MRMS provides unrivaled sensitivity and resolution, but the ionization process and detection cell provide for only a limited number of analyzed molecules. As sources of contamination are eliminated, molecules arising from urine predominately occupy the analytical volume. This limit affects MRMS measurements wherein the goal is to measure thousands of unique substances with different m/z simultaneously in a single analysis. It does not apply when the goal is to measure a smaller number of substances.

Experimental

MRMS Analysis

Mass spectral analysis was performed in an unmodified Bruker 7T-SolariX XR FTMS over the 75 to 1,000 m/z range. The LDI source was operated at 50% laser power. The LDI plate was a Protea Biosciences Redichip, nanopost array type with no chemical matrix.

Urine sample dilutions were determined by spectrophotometry over 350-360 nanometers in a Molecular Devices SpectraMax M2 spectrophotometer and carried out by adding between 0 and 50 μl of VWR Aristar Ultra-pure water, to a 5 μl urine sample, thereby adjusting the urine concentrations approximately with one another. A total of 4 μl of each diluted sample was applied to the plate and dried before analysis.

Since the Redichip is hydrophobic, pure water and urine do not readily adhere to the chip. We preferred, however, not to add methanol or acetonitrile, which is normal practice with these chips, because that would be an additional source of contamination. We found that by using a 4 μl sample and working it with the pipette tip, the drop will adhere to the engraving around the sample area and dry uniformly over the nanopost array.

A total of 200 MRMS transients were averaged together, with each 1.0 second transient generated by 500-shots of a pulsed laser directed onto a unique position on the plate as selected by means of Bruker automation. Each analysis had a cycle time of about 7 minutes. We also performed analyses with 1.5 second transients and 300 transients that had about 10 minutes and provide better resolution. Cycle time can be reduced by reducing noise in the system. The 300 transients are averaged to overcome noise in the system and the limited number of molecules being measured in each scan (about 1,000,000). If noise is reduced by 50%, only 75 transients can be used for the same result and the cycle time will be less than 2 minutes.

Calculations

In addition to the protonated molecular species, several molecular adducts, primarily with salts, were observed for most molecular species. The Na and K adduct forms (up to 3 atoms at once) were usually much more abundant than the protonated species. These, in addition to isotopic variations, yielded about 30,000 quantitatively significant mass spectral variations, providing, on average, 8 forms of each unique molecular component.

These forms were added together to provide about 4,000 distinct molecular urinary components with precisely unique masses. A total of 918 urinary substances are listed in the literature [7] as “endogenous” products of human metabolism.

Overall, 2,700 urinary substances are listed [7]. We have assigned 2,300 of these in our urinary profiles by mass alone, which are mass-consistent combinations of about 18,000 peaks in the mass spectra. We estimate that these assignments are more than 90% chemically correct. Of the 918 listed as “endogenous” [7], we found 833 that were present in analytically significant amounts in 80% or more of the urine sample mass spectra. These are listed in the on-line supplementary material. For simplicity in this initial analysis we used these 833 substances for diagnostic purposes [6].

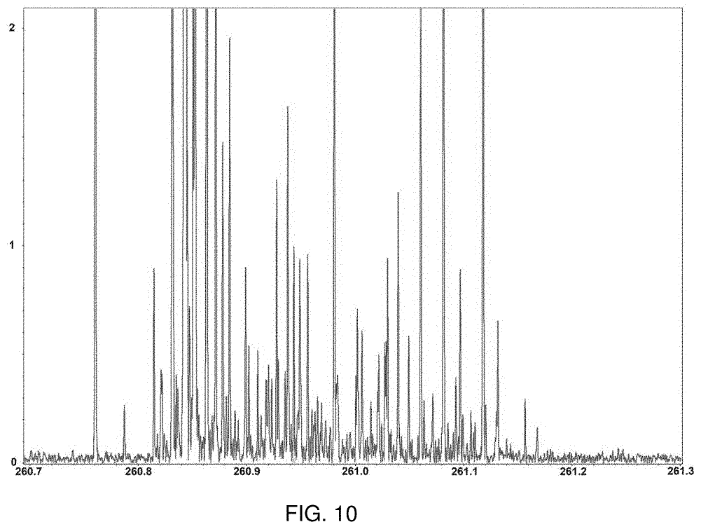

As an example, FIG. 10 shows the mass spectrum in the region of m/z=260.7 to 261.3 for a casual urine sample from a human subject, 93 years of age.

This particular sample was analyzed 12 times to distinguish actual peaks from random noise. With a criterion that the peak appeared in 10 of the 12 repeat analyses, 82,000 peaks appeared in the complete analysis, and, with a criterion that the peak appeared in 6 of the 12 analyses, 257,000 appeared. Thus, averaging out the probability of random noise, this MRMS instrument is detecting about 200,000 different m/z species at the extreme limit of its current sensitivity.

The majority of these peaks have intensities too small for useful quantitative measurement and reliable statistical analysis. For the mass range in FIG. 10, about 50 peaks were deemed sufficiently intense to include in profiling calculations. In the entire mass range, about 30,000 were present with sufficient intensity to use in the statistical profiling,

FIG. 10 shows a mass spectrum of the urine of a 93-year old human subject in the m/z 260.7 to 261.3 region. The complete MRMS mass spectrum between 75 and 1,000 amu contains 925 such regions. On average, about 200 peaks representing molecules with different m/z are detected in each such region, and we used about 35 of these in our profiles.

Results and Discussion

The analytical parameters for practical quantitative metabolic profiling used for human diagnostic purposes are different from those used in metabolic research. For profiling use at low cost, this methodology should include the measurement of a large number of molecular species suitable for detection and quantization of many different health conditions in a single analysis.

It is, however, unnecessary for the measured substances to have known links to the pathologies being detected. By virtue of their presence within the metabolism, the concentrations of many molecular species are affected by processes in which they may play a small or even insignificant part. These correlations can be statistically added together to provide useful health information.

For example, many urinary amino acids and amines are correlated with human sex, even though direct links between these substances and sex are presently unknown [6, 12]. Similarly, about 30% of the metabolic substances in human urine are correlated with age, even though the fundamental causes of physiological aging are only dimly understood [5-6].

While the diagnostic use of the technique described herein has, so far, been limited to substances expected to be involved in normal human metabolism [6], we observed many other substances that were disease correlated, some of which are probably not metabolites. By virtue of their presence in human tissues, their concentrations have been affected by human disease, and they could be useful in detecting and predicting such disease.

A foreign substance, (e.g., Ibuprofen or any substance that is effective), tracers or markers, can be introduced that establishes useful urinary or other tissue health correlations. A foreign substance could be, for example various non-naturally occurring drugs. Examples are numerous, but would include drugs like Ibuprofen, Droxidopa, Lidocaine, etc. A foreign substance can be introduced by diet, drugs administered by any suitable route known in the art or by other methods of introduction known in the art. These, too, could be detected and measured by magnetic resonance mass spectrometry.

In any case, the procedure that we have developed, as described herein, and applied to metabolic profiling permits the quantitative measurement of a very large number of urinary substances. Those measurements have already been used for the detection and quantization of several useful aspects of human health [6].

Conclusions

The method that we describe in this example is suitable for use of MRMS for quantitative metabolic profiling. By means of nanopost array LDI and minimalist sample manipulation, made possible by the very high resolution of MRMS, we have eliminated contamination and background noise sufficiently to allow the necessary urinary molecular profiles to be seen and measured effectively.

Advances in mass spectrometry have revolutionized biochemistry and human metabolomics, which will gradually lead to understandable biochemical models and many reasoned medical advances.

In the mean-time, the empirical use of high resolution mass spectrometry and careful sampling as illustrated in this example and in Example 1 [6] can make important contributions to the quality and length of human life.

For example, a $2 kit for submitting a urine sample can be acquired by a consumer. The kit contains a suitable MALDI fabric matrix on which the user places a drop of urine, allows it to dry, and then mails the fabric to a central mass spectrometry laboratory inn an envelope with a 50-cent stamp. Including automated analysis and with appropriate software, the consumer could receive valuable information about his or her current and probable future health for a total cost of perhaps $5, and within a few days.

MRMS technology thus makes valuable contributions in preventive, diagnostic, and therapeutic medicine available to all people, regardless of their social and economic circumstances.

At present, many people do not have the opportunity to live for an entire intrinsic human life span. One of the aims of the method disclosed herein is to provide more people with markedly increased quality and length of life.

### REFERENCES

1. Marshall, A. G. and Chen, T.; 40 Years of Fourier Transform Ion Cyclotron Resonance Mass Spectrometry. International Journal of Mass Spectrometry 377, 410-420 (2015)
2, Robinson, N. E. and Robinson, A, B.; Origins of Metabolic Profiling. In: Metz, T. O. (ed) Metabolic Profiling, pp 1-24, Springer Protocols, Methods in Molecular Biology 708, Humana Press (2011)
3. Robinson, A. B. and Pauling, L. C.; Techniques of Orthomolecular Diagnosis. Clinical Chemistry 20, 961-965 (1974)
4. Robinson, A. B., Dirren, H., Sheets, A., Miquel, J. and Lundgren, P. R.; Quantitative Aging Pattern in Mouse Urine Vapor as Measured by Gas-Liquid Chromatography. Experimental Gerontology 11, 11-16 (1976)
5. Robinson, A. B. and Robinson, L. R.; Mechanisms of Ageing and Development 59, 47-67 (1991)
6. Example 1
7. Bouatra, S. Mandal, R., Guo, A. C., Wilson, M. R., et al. In: The Human Urine Metabolome, The Metabolomics Innovation Centre (2013)
8. 11. Lei, Z., Huhman, D. V., and Sumner, L. V.; Mass Spectrometry Strategies in Metabolomics. J. Biological Chemistry 286, 25435-25442 (2011) and Brown, S. C., Kruppa, G., and Dasseux, J. L.; Metabolomics Applications of FT-ICR Mass Spectrometry. Mass Spectrometry Rev 24, 223-31 (2005)
9. Fenn, J. B., Mann, M., Meng, C. K., Wong, S. F., and Whitehouse, C. M.; Electrospray Ionization for Mass Spectrometry of Large Biomolecules. Science 246, 64-71 (1989)
10, Tanaka, K., Waki, H., Ido, Y., Akita, S., Yoshida, Y., Yoshida, T. and Matsuo, T.; Protein and Polymer Analyses Up to 100,000 by Laser Ionization Time-of-flight Mass Spectrometry. Rapid Communications inn Mass Spectrometry 2, 151-153 (1988)
11. Karas, M., Bachmann, D., and Hillenkamp, F.; Influence of Wavelength in High-Irradiance Ultraviolet Laser Desorption Mass Spectrometry of Organic Molecules. Analytical Chemistry 57, 2935-2939 (1985)
12, Dirren, H., Robinson, A. B., and Pauling, L. C.; Sex-Related Patterns in the Profiles of Human Urinary Amino Acids. Clinical Chemistry 21, 1970-1975 (1975)

### APPENDIX

Appendix 1. Metabolic Profiling with Magnetic Resonance Mass Spectrometry and a Human Urine Bank: Profiles for Aging, Sex, Heart Disease, Breast Cancer and Prostate Cancer Noah Robinson, Ph.D. Matthew Robinson, Ph.D. Arthur Robinson, Ph.D. Journal of American Physicians and Surgeons Volume 22 Number 3 Fall 2017, 75-84.

Additional details of the above described embodiments are set forth in the appendix or appendices referred to hereinabove in the section entitled “Reference to Appendix,” which appendix or appendices are attached hereto and form part of the Detailed Description of this patent application.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A method for constructing a metabolic profile, the method comprising:
obtaining a urine sample from a mammalian subject;
diluting the sample solely with ultra-pure water to a concentration suitable for mass spectrometry;
obtaining a mass spectrum of the urine sample, wherein obtaining the mass spectrum comprises:
subjecting the urine sample to magnetic resonance mass spectrometry (MRMS),
determining mass spectrometric peaks in the mass spectrum present in analytically significant amounts,
identifying, among the plurality of mass spectrometric peaks in the mass spectrum present in analytically significant amounts, mass spectrometric peaks representing a plurality of urinary substances of interest, wherein identifying the plurality of urinary substances of interest comprises:
identifying all mass spectrometric peaks present in analytically significant amounts in the mass spectrum that represent:
each urinary substance of interest in the plurality,
each isotope of each urinary substance of interest in the plurality,
each adduct of each urinary substance of interest in the plurality, and/or
each variant of each urinary substance of interest in the plurality;
quantitatively measuring the amount of each urinary substance of interest in the plurality by summing, for each urinary substance of interest in the plurality, the mass spectrometric peaks representing:
each urinary substance of interest in the plurality,
each isotope of each urinary substance of interest in the plurality,
each adduct of each urinary substance of interest in the plurality, and/or
each variant of each urinary substance of interest in the plurality; and
performing statistical calculations to determine a diagnostically useful profile by determining what combinations and/or amounts of urinary substances of interest in the plurality correlate with a disease or condition of interest, thereby constructing a metabolic profile of the disease or condition of interest in the subject.

2. The method of claim 1, wherein the mammalian subject is a human subject.

3. The method of claim 1, wherein MRMS is performed in positive ion mode.

4. The method of claim 1, wherein the MRMS comprises laser desorption ionization (LDI).

5. The method of claim 1, wherein the MRMS is matrix assisted laser desorption ionization (MALDI)-MRMS.

6. The method of claim 1, wherein the urinary substance of interest is of mammalian metabolic origin.

7. The method of claim 1, further comprising identifying the plurality of substances of human metabolic origin present in the urine sample that are also present in at least 80% of metabolic profiles in a database of metabolic profiles of human urine samples.

8. The method of claim 4, wherein the urine sample is introduced onto a nanopost array ionization plate or a nanopost matrix and the LDI is performed from the nanopost array ionization plate.

9. The method of claim 1, wherein the MRMS is electrospray (ESI)-MRMS.

10. The method of claim 1, wherein the sample is diluted with ultra-pure water only.

11. The method of claim 1, wherein mass spectral analysis is performed over a range from 75 to 1,000 m/z.

12. The method of claim 1, wherein at least one of the urinary substances of interest in the plurality is selected from the urinary substances listed in Table 2.

13. The method of claim 1, wherein the volume of the urine sample is 0.1-1 μl, 1-10 μl, 10-20 μl, 20-30 μl, 30-40 μl, or 40-50 μl.

14. The method of claim 1, wherein the volume of the urine sample is 5 μl.

15. The method of claim 1, wherein the urine sample is subjected to a run of magnetic resonance mass spectrometry (MRMS) in positive ion mode of a duration of 0.1 min-1 min, 1 min-5 min, 5 min-10 min, 10 min-30 min, or 30 min-60 min.

16. The method of claim 1, wherein the urine sample is subjected to a series of runs of magnetic resonance mass spectrometry (MRMS) in positive ion mode of a total duration of 2 min.

17. The method of claim 1, wherein the mass spectrometric peaks with specific molecular masses present in analytically significant amounts in the urine sample are identified by their exact masses.

18. The method of claim 1, wherein the disease or condition of interest is cardiovascular disease, cardiac illness, prostate cancer or breast cancer.

19. The method of claim 1, wherein the disease or condition of interest is selected from a disease or condition of interest listed in Table 1.

20. A method for assessing the progression of a disease or condition of interest in a mammalian patient during a time period of interest comprising:
obtaining a metabolic profile from a urine sample from the patient at selected sequential time points in the time period of interest;
determining the amounts of:
urinary substances of interest for monitoring for the progression of a disease of interest in its metabolic profile;
calculating the change in amount of urinary substances of interest among each of the selected sequential time points in the time period of interest;
calculating, with successive strength of each metabolic profile obtained at a selected sequential time point in the time period of interest, the progress of the patient's illness as a function of time and treatment, wherein the calculating comprises determining a diagnostic coefficient for the condition of interest;
determining:
which parameters are indicative that the disease is progressing in the patient;
which parameters are indicative that the disease is not progressing in the patient;
which parameters are indicative that the disease is diminishing or that the patient's health is improving, and
if the disease is progressing in the patient, administering a drug or treatment to ameliorate, reverse or stop the progression of the disease; or
if the disease is not progressing in the patient, modulating therapy appropriately.

21. The method of claim 20, wherein the mammalian patient is a human patient.

22. The method of claim 20, wherein the disease or condition of interest is cardiovascular disease, cardiac illness, prostate cancer, or breast cancer.

23. The method of claim 20, wherein the disease or condition of interest is selected from a disease or condition of interest listed in Table 1.

24. A method for assessing the presence and amounts of at least one urinary substance of interest in a mammalian urine sample, the method comprising:
obtaining a urine sample from a mammalian patient;
diluting the sample solely with ultra-pure water to a concentration suitable for mass spectrometry;
obtaining a mass spectrum of the urine sample, wherein obtaining the mass spectrum comprises:
subjecting the urine sample to magnetic resonance mass spectrometry (MRMS),
identifying mass spectrometric peaks in the mass spectrum present in analytically significant amounts, wherein the identifying mass spectrometric peaks comprises:
performing a statistical evaluation to demonstrate existence of a metabolic profile, and
testing the metabolic profile by a diagnostic coefficient method,
identifying at least one urinary substance of interest among a plurality of urinary substances in the urine sample, wherein identifying the at least one urinary substance of interest comprises identifying all mass spectrometric peaks in the mass spectrum representing the at least one urinary substance of interest, isotopes of the at least one urinary substance of interest, adducts of the at least one urinary substance of interest, and/or other variants of the at least one urinary substance of interest;
quantitatively measuring the amount of the at least one urinary substance of interest by summing the mass spectrometric peaks in the plurality comprising:
identifying the isotopic peak of all molecular ions of the urinary substance of interest,
identifying the isotopic peak of all molecular ion adducts of the urinary substance of interest,
identifying the isotopic peaks of a molecular ion variants of the urinary substance of interest,
combining these peaks to determine the amount of the urinary substance of interest.

25. The method of claim 24, wherein the mammalian patient is a human patient.

26. The method of claim 24, wherein MRMS is performed in positive ion mode.

27. The method of claim 24, wherein the MRMS comprises laser desorption ionization (LDI).

28. The method of claim 24, wherein the MRMS is matrix assisted laser desorption ionization (MALDI)-MRMS.

29. The method of claim 24, wherein the urinary substance of interest is of mammalian metabolic origin.

30. The method of claim 24, further comprising identifying a plurality of substances of mammalian metabolic origin present in the urine sample that are also present in at least 80% of metabolic profiles in a database of metabolic profiles of mammalian urine samples.

31. The method of claim 27, wherein the urine sample is introduced onto a nanopost array ionization plate and the LDI is performed from the nanopost array ionization plate.

32. The method of claim 24, wherein the MRMS is electrospray (ESI)-MRMS.

33. The method of claim 24, wherein the sample is diluted with ultra-pure water only.

34. The method of claim 24, wherein the urinary substance of interest is of mammalian metabolic origin.

35. The method of claim 24, wherein mass spectral analysis is performed over a range from 75 to 1,000 m/z.

36. The method of claim 24, wherein a plurality of urinary substances of interest are assessed.

37. The method of claim 36, wherein the plurality of urinary substances of interest is selected from the urinary substances listed in Table 2.

38. The method of claim 24, wherein the volume of the urine sample is 0.1-1 μl, 1-10 μl, 10-20 μl, 20-30 μl, 30-40 μl, or 40-50 μl.

39. The method of claim 24, wherein the volume of the urine sample is 5 μl.

40. The method of claim 24, wherein the urine sample is subjected to a run of magnetic resonance mass spectrometry (MRMS) in positive ion mode of a duration of 0.1 min-1 min, 1 min-5 min, 5 min-10 min, 10 min-30 min, or 30 min-60 min.

41. The method of claim 24, wherein the urine sample is subjected to a series of runs of magnetic resonance mass spectrometry (MRMS) in positive ion mode of a duration of 2 min.

42. The method of claim 24, wherein the mass spectrometric peaks with specific molecular masses present in analytically significant amounts in the urine sample are identified by their exact masses.

43. A method for monitoring a change in amount of at least one urinary substance of interest in a mammalian urine sample during a time period of interest, the method comprising:
obtaining a urine sample from a mammalian patient;
selecting a plurality of sequential time points at which to measure an amount of a selected urinary substance of interest in the urine sample,
performing the method of claim 24 at a first selected time point at the beginning of the time period of interest;
performing the method of claim 24 at each of the selected subsequent sequential time points in the time period of interest;
calculating the changes in amounts of the at least one urinary substance of interest for each sequential time point of the plurality of sequential time points during the selected time period by comparing the amount of the at least one selected urinary substance of interest at the first selected time point to the amount of the at least one selected urinary substance of interest at the selected subsequent sequential time points of the plurality wherein the calculating comprises determining a diagnostic coefficient.

44. The method of claim 43, wherein the mammalian urine sample is a human urine sample and the mammalian patient is a human patient.
